(19) United States
(12) Patent Application Publication
(10) Pub. No.: US 2023/0048361 A1
(43) Pub. Date: Feb. 16, 2023

(54) METHODS OF CULTURING T CELLS AND USES OF SAME

(71) Applicants: KAHR Medical Ltd., Jerusalem (IL); Beth Israel Deaconess Medical Center, Inc., Boston, MA (US)

(72) Inventors: Senthil Kumar MUTHUSWAMY, Boston, MA (US); Manuel HIDALGO MEDINA, Boston, MA (US); Qingda MENG, Malden, MA (US); Yaron PEREG, Shoham (IL); Ayelet CHAJUT, Ramat HaSharon (IL)

(73) Assignees: KAHR Medical Ltd., Jerusalem (IL); Beth Israel Deaconess Medical Center, Inc., Boston, MA (US)

(21) Appl. No.: 17/788,307

(22) PCT Filed: Dec. 30, 2020
(86) PCT No.: PCT/IL2020/051356

§ 371 Date: Jun. 23, 2022

Related U.S. Application Data
(60) Provisional application No. 62/955,463, filed on Dec. 31, 2019.

(51) Int. Cl. C12N 5/0783; A61K 35/17

(52) U.S. Cl. CPC C12N 5/0636; A61K 35/17; C12N 2501/2302; C12N 2501/2315; C12N 2501/2321; C12N 2501/998

(57) ABSTRACT

Methods of culturing T cells are provided. Accordingly there is provided a method of culturing T cells comprising adding to immune cells comprising T cells obtained from a subject having a pathology a non-cellular agent capable of binding 4-1BB and activating said 4-1BB signaling pathway; and culturing the immune cells with said agent for more than 7 days. Also provided are T cells obtainable by the method and uses thereof.

### RELATED APPLICATION/S

This application claims the benefit of priority under 35 USC § 119(e) of U.S. Provisional Patent Application No. 62/955,463 filed 31 Dec. 2019, the contents of which are incorporated herein by reference in their entirety.

### SEQUENCE LISTING STATEMENT

The ASCII file, entitled 85494 Sequence Listing, created on Dec. 28, 2020, comprising 241,664 bytes, submitted concurrently with the filing of this application is incorporated herein by reference.

### BACKGROUND OF THE INVENTION

The present invention, in some embodiments thereof, relates to methods of culturing T cells and uses of same.

Cell-based adoptive immune therapies involving in-vitro activation and expansion of T cells hold promise for the treatment of various diseases. For example, adoptive transfer of antigen-specific CD8⁺ T cells can be used for the treatment of malignancies and infections, while specific regulatory T cells (Tregs) can be harnessed for suppression of autoimmune processes. A major challenge for T cell based immune therapies is the necessity to identify T cells specifically reactive against cells associated with the disease of interest.

4-1BB (CD137) is a member of the TNF receptor super family that functions as a costimulatory molecule promoting proliferation and survival of activated T cells. 4-1BB activation upregulates survival genes, enhances cell division, induces cytokine production and prevents activation induced cell death in T-cells.

Expression of 4-1BB on T cells is transient and is limited to T cells that have recently been activated by T cell receptor (TCR) engagement and signaling. Consequently, upregulation of CD137 on recently activated T cells has been used to identify and isolate virus- and tumor-reactive T cells (e.g. Qunrui Ye et al. (2014) Clin Cancer Res. 2014 Jan. 1; 20(1): 44-55; Sivan Seliktar-Ofir (2017) Front. Immunol. 8:1211; Wolf et al. (2007) Blood. 110: 201-210; Parkhurst et al. (2017) Clin Cancer Res. 23(10): 2491-25; Tan et al., (2019) Journal for ImmunoTherapy of Cancer, volume 7, Article number: 232; and EP Patent Application Publication No. EP3118322).

4-1BBL is the activating ligand of 4-1BB. 4-1BBL naturally forms a homo-trimer but signaling via 4-1BB requires significant oligomerization of 4-1BBL. 4-1BBL is present on a variety of antigen presenting cells (APCs), including dendritic cells (DCs), B cells, and macrophages.

Activating T cells in the presence of agonistic 4-1BB binding agents such as antibodies and 4-1BBL polypeptides has been suggested in the art (e.g. International Patent Applications Publication Nos. WO2003049755; WO2018/127917; and WO2018/127919).

Additional Background art includes International Patent Applications Publication Nos. WO2017182672 and WO2015039100.

### SUMMARY OF THE INVENTION

According to an aspect of some embodiments of the present invention there is provided a method of culturing T cells, the method comprising:
(a) adding to immune cells comprising T cells obtained from a subject having a pathology a non-cellular agent capable of binding 4-1BB and activating the 4-1BB signaling pathway; and
(b) culturing the immune cells with the agent for more than 7 days.

According to some embodiments of the invention, the culturing is effected for more than 10 days.

According to some embodiments of the invention, the culturing is effected for about 14 days.

According to some embodiments of the invention, the method being effected without isolation of 4-1BB positive cells prior to the (a) and/or the (b).

According to some embodiments of the invention, the method being effected without isolation of 4-1BB positive cells during and/or following the (b).

According to some embodiments of the invention, the method effected without isolation of the T cells from the immune cells prior to the (a) and/or the (b).

According to some embodiments of the invention, the method comprising adding at least one cytokine to the immune cells in step (a) and culturing the immune cells with the cytokine in step (b).

According to some embodiments of the invention, the method further comprising pre-culturing the immune cells with at least one cytokine prior to the (a).

According to some embodiments of the invention, the pre-culturing is effected until the percentage of the T cells in the immune cells is at least 80%.

According to some embodiments of the invention, the pre-culturing is effected for 2-14 days.

According to some embodiments of the invention, the pre-culturing is effected for 7-14 days.

According to some embodiments of the invention, the method being effected without isolation of the T cells from the immune cells prior to the pre-culturing.

According to some embodiments of the invention, the method being effected without adding to the immune cells a T cell stimulatory agent capable of at least transmitting a primary activating signal to the T cells.

According to some embodiments of the invention, the culturing is effected under conditions which allow enrichment of T cell clones reactive to cells associated with the pathology.

According to some embodiments of the invention, the immune cells are obtained from a blood sample of the subject.

According to some embodiments of the invention, the immune cells comprise PBMCs.

According to some embodiments of the invention, the immune cells are obtained from a tissue comprising cells associated with the pathology.

According to some embodiments of the invention, the at least one cytokine is selected from the group consisting of IL-2, IL-15, IL-21, IL-12 and IL-7.

According to some embodiments of the invention, the at least one cytokine comprises IL-2, IL-15 and IL-21.

According to some embodiments of the invention, the non-cellular agent is capable of binding a ligand or receptor of a type I membrane protein.

According to some embodiments of the invention, the (a) further comprises adding a non-cellular agent capable of binding a ligand or receptor of a type I membrane protein.

According to some embodiments of the invention, the type I membrane protein is PD1.

According to some embodiments of the invention, the type I membrane protein is SIRPα.

According to some embodiments of the invention, the non-cellular agent is soluble.

According to some embodiments of the invention, the non-cellular agent is immobilized.

According to some embodiments of the invention, the non-cellular agent is an antibody.

According to some embodiments of the invention, the non-cellular agent capable of binding 4-1BB and activating the 4-1BB signaling pathway is a polypeptide comprising an amino acid sequence of 4-1BBL.

According to some embodiments of the invention, the non-cellular agent is a fusion polypeptide.

According to some embodiments of the invention, the fusion polypeptide comprises an amino acid sequence of 4-1BBL and an amino acid sequence of PD1.

According to some embodiments of the invention, the fusion polypeptide comprises an amino acid sequence having at least 80% identity to SEQ ID NO: 49.

According to some embodiments of the invention, the fusion polypeptide comprises SEQ ID NO: 49 or as set forth in SEQ ID NO: 49.

According to some embodiments of the invention, the fusion polypeptide comprises an amino acid sequence having at least 80% identity to SEQ ID NO: 49 or 74.

According to some embodiments of the invention, the fusion polypeptide comprises SEQ ID NO: 49 or 74 or as set forth in SEQ ID NO: 49 or 74.

According to some embodiments of the invention, the fusion polypeptide comprises an amino acid sequence of 4-1BBL and an amino acid sequence of SIRPα.

According to some embodiments of the invention, the fusion polypeptide comprises an amino acid sequence having at least 80% identity to SEQ ID NO: 89.

According to some embodiments of the invention, the fusion polypeptide comprises SEQ ID NO: 89 or as set forth in SEQ ID NO: 89.

According to some embodiments of the invention, the method further comprising expanding the T cells following the (b).

According to some embodiments of the invention, the method further comprising isolating the T cells from the immune cells following the (b) and/or the expanding.

According to some embodiments of the invention, the method further comprising determining a sequence of a T cell receptor (TCR) expressed by at least one of the T cells following the (b), the expanding or the isolating.

According to some embodiments of the invention, the method further comprising transducing a T cell with a nucleic acid sequence encoding the TCR.

According to some embodiments of the invention, the method further comprising transducing the T cells with a nucleic acid sequence encoding a chimeric antigen receptor (CAR) following the (b), the expanding or the isolating.

According to some embodiments of the invention, the method comprising adoptively transferring the immune cells and/or the T cells following the (b), the expanding or the transducing to a subject in need thereof.

According to an aspect of some embodiments of the present invention there is provided T cells obtainable by the method.

According to an aspect of some embodiments of the present invention there is provided the T cells for use in adoptive cell therapy to a subject in need thereof.

According to an aspect of some embodiments of the present invention there is provided a method of treating a pathology that can benefit from adoptive T cell therapy in a subject in need thereof, the method comprising administering to the subject a therapeutically effective amount of the T cells of claim 36, thereby treating the pathology.

According to some embodiments of the invention, the immune cells and/or the T cells are autologous to the subject in need thereof.

According to some embodiments of the invention, the pathology is selected from the group consisting of a hyper-proliferative disease, a disease associated with immune suppression and infection.

According to some embodiments of the invention, the pathology is cancer.

According to some embodiments of the invention, the cancer is selected from the group consisting of gastrointestinal (GI) cancer, breast cancer, ovarian cancer and pancreatic cancer.

According to specific embodiments, the cancer is selected from the group consisting of pancreatic cancer, lung cancer, colon cancer and leukemia.

According to some embodiments of the invention, the subject is human.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 shows dot-plot FACS analysis demonstrating percentages of CD8 and CD4 T cells following initial 10 days culturing period of PBMCs obtained from four pancreatic cancer patients in a media containing hIL2, hIL-15 and hIL-21.

FIG. 2 demonstrates IFNγ levels in the culture media during a second 14 days culturing period of PBMCs obtained from two pancreatic cancer patients in the presence of PD1-4-1BBL fusion protein (marked as DSP105, SEQ ID NO49), as compared to cell cultured under the same conditions without the fusion protein.

FIG. 3 demonstrates the percentage of three subpopulations of memory T cells from the total PBMC population following the second 14 days culturing period of PBMCs obtained from a pancreatic cancer patient in the presence of PD1-4-1BBL fusion protein (marked as DSP105, SEQ ID NO: 49), or with organoids as compared to control cells cultured under the same conditions without the fusion protein or the organoid.

FIG. 4 shows representative microscope images demonstrating the effect of a PD1-4-1BBL fusion protein (marked as DSP105-G3, SEQ ID NO: 49, or DSP105-V10, SEQ ID NO: 74) on expansion of T-cell clusters obtained from a non-small cell lung cancer (NSCLC) patient (OL #1, Group 1, day 8 following treatment).

### DESCRIPTION OF DETAILED EMBODIMENTS OF THE INVENTION

The present invention, in some embodiments thereof, relates to methods of culturing T cells and uses of same.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Cell-based adoptive immune therapies, involving in-vitro activation and expansion of T cells, can be used for the treatment of various diseases including malignancies, infections and autoimmune diseases. A major challenge for T cell based immune therapies is the necessity to identify T cells specifically reactive against cells associated with the disease of interest.

Whilst reducing specific embodiments of the present invention to practice, the present inventors have now devised an ex-vivo environment for the clonal selection of cancer-specific T cells having a memory phenotype by culturing the T cells in the presence of a PD1-4-1BBL fusion protein.

Thus, according to a first aspect of the present invention, there is provided a method of culturing T cells, the method comprising:

(a) adding to immune cells comprising T cells obtained from a subject having a pathology a non-cellular agent capable of binding 4-1BB and activating said 4-1BB signaling pathway; and

(b) culturing said immune cells with said agent for more than 7 days.

As used herein, the term “T cells” refers to a differentiated lymphocyte with a CD3+, T cell receptor (TCR)+having either CD4+ or CD8+ phenotype. The T cell may be either an effector or a regulatory T cell.

According to specific embodiments, the T cells are effector T cells

As used herein, the term “effector T cells” refers to a T cell that activates or directs other immune cells e.g. by producing cytokines or has a cytotoxic activity e.g., CD4+, Th1/Th2, CD8+ cytotoxic T lymphocyte.

According to specific embodiments, the T cells are regulatory T cells.

As used herein, the term “regulatory T cell” or “Treg” refers to a T cell that negatively regulates the activation of other T cells, including effector T cells, as well as innate immune system cells. Treg cells are characterized by sustained suppression of effector T cell responses. According to a specific embodiment, the Treg is a CD4+CD25+Foxp3+ T cell.

According to specific embodiments, the T cells are CD4+ T cells.

According to other specific embodiments, the T cells are CD8+ T cells.

According to specific embodiments, the T cells comprise memory T cells. Non-limiting examples of memory T cells include effector memory CD4+ T cells with a CD3+/CD4+/CD45RA−/CCR7− phenotype, central memory CD4+ T cells with a CD3+/CD4+/CD45RA−/CCR7+ phenotype, effector memory CD8+ T cells with a CD3+/CD8+CD45RA−/CCR7− phenotype, central memory CD8+ T cells with a CD3+/CD8+CD45RA−/CCR7+ phenotype, tissue-resident memory T cells with a CD3+/CD45RO+/CD69+/CD103+ or a CD3+/CD45RO+/CD69+/CD103− phenotype, or effector memory T cells with CD3+/CD45RO+/CD69−/CCR7− phenotype.

According to specific embodiments, the T cells comprise activated cells. Non-limiting examples of activated T cells include effector activated CD4+ cells and, effector activated CD8+ T cells with a tissue resident memory or effector memory phenotype.

According to specific embodiments, the T cells comprise cells expressing 4-1BB.

According to specific embodiments, the immune cells comprise at least 70%, at least 75%, at least 80%, at least 85%, at least 90%, or at least 95% T cells.

According to specific embodiments, the immune cells comprise 45-70% T cells.

According to specific embodiments, the immune cells comprise at least 70% T cells.

According to specific embodiments, the immune cells comprise at least 80% T cells.

Thus, according to specific embodiments, the immune cells comprise cells other than the T cells.

According to specific embodiments, the immune cells comprise phagocytic cells.

As used herein, the term “phagocytic cells” refer to a cell that is capable of phagocytosis and include both professional and non-professional phagocytic cells. Methods of analyzing phagocytosis are well known in the art and include for examples killing assays, flow cytometry and/or microscopic evaluation (live cell imaging, fluorescence microscopy, confocal microscopy, electron microscopy). According to specific embodiments, the phagocytic cells are selected from the group consisting of monocytes, dendritic cells (DCs) and granulocytes.

According to specific embodiments, the immune cells comprise monocytes.

According to specific embodiments, the term “monocytes” refers to both circulating monocytes and to macrophages (also referred to as mononuclear phagocytes) present in a tissue.

According to specific embodiments, the monocytes comprise macrophages. Typically, cell surface phenotype of macrophages include CD14, CD40, CD11b, CD64, F4/80 (mice)/EMR1 (human), lysozyme M, MAC-1/MAC-3 and CD68.

According to specific embodiments, the monocytes comprise circulating monocytes.

Typically, cell surface phenotypes of circulating monocytes include CD14 and CD16 (e.g. CD14++CD16−, CD14+CD16++, CD14++CD16+).

According to specific embodiments, the immune cells do not comprise monocytes.

According to specific embodiments, the immune cells comprise DCs.

As used herein the term “dendritic cells (DCs)” refers to any member of a diverse population of morphologically similar cell types found in lymphoid or non-lymphoid tissues. DCs are a class of professional antigen presenting cells, and have a high capacity for sensitizing HLA-restricted T cells. DCs include, for example, plasmacytoid dendritic cells, myeloid dendritic cells (including immature and mature dendritic cells), Langerhans cells, interdigitating cells, follicular dendritic cells. Dendritic cells may be recognized by function, or by phenotype, particularly by cell surface phenotype. These cells are characterized by their distinctive morphology having veil-like projections on the cell surface, intermediate to high levels of surface HLA-class II expression and ability to present antigen to T cells, particularly to naive T cells (See Steinman R, et al., Ann. Rev. Immunol. 1991; 9:271-196.). Typically, cell surface phenotype of DCs include CD1a+, CD4+, CD86+, or HLA-DR. The term DCs encompasses both immature and mature DCs.

According to specific embodiments, the immune cells do not comprise DCs.

According to specific embodiments, the immune cells comprise granulocytes.

As used herein, the term “granulocytes” refer to polymorphonuclear leukocytes characterized by the presence of granules in their cytoplasm.

According to specific embodiments, the granulocytes comprise neutrophils.

According to specific embodiments, the granulocytes comprise mast-cells.

According to specific embodiments, the immune cells comprise B cells.

As used herein the term “B cells” refers to a lymphocyte with a B cell receptor (BCR)+, CD19+ and or B220+ phenotype. B cells are characterized by their ability to bind a specific antigen and elicit a humoral response.

According to specific embodiments, the immune cells comprise NK cells.

As used herein the term “NK cells” refers to differentiated lymphocytes with a CD16+CD56+ and/or CD57+ TCR− phenotype. NK are characterized by their ability to bind to and kill cells that fail to express “self” MHC/HLA antigens by the activation of specific cytolytic enzymes, the ability to kill tumor cells or other diseased cells that express a ligand for NK activating receptors, and the ability to release protein molecules called cytokines that stimulate or inhibit the immune response.

According to specific embodiments, the immune cells comprise NKT cells.

As used herein the term “NKT cells” refers to a specialized population of T cells that express a semi-invariant αβ T-cell receptor, but also express a variety of molecular markers that are typically associated with NK cells, such as NK1.1. NKT cells include NK1.1+ and NK1.1−, as well as CD4+, CD4−, CD8+ and CD8− cells. The TCR on NKT cells is unique in that it recognizes glycolipid antigens presented by the MHC I-like molecule CD1d. NKT cells can have either protective or deleterious effects due to their abilities to produce cytokines that promote either inflammation or immune tolerance.

According to specific embodiments, the immune cells comprise antigen presenting cells (APCs).

As used herein, the term “antigen presenting cell (APC)” refers to an immune cell capable of internalizing and processing an antigen, so that antigenic determinants are presented on the surface of the cell as MHC-associated complexes, in a manner capable of being recognized by T cells. Non-limiting examples of APC include dendritic cells, mononuclear cells (e.g. macrophages) and B cells.

According to specific embodiments, the immune cells comprise less than 30%, less than 20%, less than 10%, less than 5% APCs.

According to specific embodiments, the immune cells do not comprise APCs.

Methods of obtaining immune cells comprising T cells from a subject are well known in the art.

For example, according to specific embodiments, the immune cells are obtained from a blood sample of the subject.

Thus, according to specific embodiments, the immune cells comprise peripheral mononuclear blood cells (PBMCs).

As used herein the term “peripheral mononuclear blood cells (PBMCs)” refers to a blood cell having a single nucleus and includes lymphocytes, monocytes and dendritic cells (DCs).

According to specific embodiments, the PBMCs are selected from the group consisting of dendritic cells (DCs), T cells, B cells, NK cells and NKT cells.

According to specific embodiments, the PBMCs comprise T cells, B cells, NK cells and NKT cells.

Methods of obtaining PBMCs are well known in the art, such as drawing whole blood from a subject and collection in a container containing an anti-coagulant (e.g. heparin or citrate); and apheresis. Following, the PBMCs are purified from the peripheral blood. There are several methods and reagents known to those skilled in the art for purifying PBMCs from whole blood such as leukapheresis, sedimentation, density gradient centrifugation (e.g. ficoll), centrifugal elutriation, fractionation and chemical lysis of e.g. red blood cells (e.g. by ACK). Such methods are described for example in THE HANDBOOK OF EXPERIMENTAL IMMUNOLOGY, Volumes 1 to 4, (D. N. Weir, editor) and FLOW CYTOMETRY AND CELL SORTING (A. Radbruch, editor, Springer Verlag, 2000).

According to other specific embodiments, the immune cells are obtained from a tissue comprising cells associated with the pathology. Methods for obtaining a tissue sample from a subject are well known in the art and include, but are not limited to biopsy.

Thus, according to specific embodiments, the immune cells comprise tumor infiltration immune cells”.

As used herein the term “tumor infiltrating immune cells” refer to white blood cells that have left the bloodstream and migrated into a tumor, and include both mononuclear and polymorph nuclear immune cells.

According to specific embodiments, the tumor infiltrating immune cells are selected from the group consisting of T cells, B cells, NK cells, macrophages, neutrophils, dendritic cells, mast cells, eosinophils and basophils.

According to specific embodiments, the immune cells comprise tumor infiltrating lymphocytes.

As used herein the term “tumor infiltrating lymphocytes (TILs) refers to mononuclear white blood cells that have left the bloodstream and migrated into a tumor.

According to specific embodiments, the TILs are selected from the group consisting of T cells, B cells and NK cells.

Methods of obtaining TILs are well known in the art, such as obtaining tumor samples from a subject by e.g. biopsy, surgery or necropsy and preparing a single cell suspension thereof. The single cell suspension can be obtained in any suitable manner, e.g., mechanically (disaggregating the tumor using, e.g., a gentleMACS™ Dissociator, Miltenyi Biotec, Auburn, Calif.) or enzymatically (e.g., collagenase or DNase). Following, the TILs can be purified from the cell suspension by e.g. density gradient centrifugation (e.g. ficoll). Such methods are described for example in Qunrui Ye et al. (2014) Clin Cancer Res. 2014 Jan. 1; 20(1): 44-55; Sivan Seliktar-Ofir (2017) Front. Immunol. 8:1211; and Besser et al. (2010) Clin Cancer Res. 16(9): 2646-55.

Regardless of the procedure employed, according to specific embodiments once immune cells are obtained from the subject, specific immune cell types comprising T cells may be further isolated.

As used herein the term “isolating” refers to an active process of purifying a specific immune cell type(s) (e.g. T cell, 4-1BB⁺ cell) from other immune cells comprised in a given immune cell sample. The result of the “isolation” process is an “isolated” immune cell type(s). The term “isolating” does not refer to enrichment of a specific immune cell population(s) due to culture conditions, also known as biological selection. According to specific embodiments, isolating refers to immune-isolating.

Thus, according to specific embodiments, the method is effected on T cells isolated from immune cells obtained from the subject.

According to specific embodiments, the method comprises isolating the T cells from the immune cells obtained from the subject prior to adding the non-cellular agent capable of binding 4-1BB and activating 4-1BB signaling pathway and/or culturing in the presence of same.

There are several methods and reagents known to those skilled in the art for purifying the desired immune cell, such as selection of specific cell types using cell surface markers (using e.g. FACS sorter or magnetic cell separation techniques such as are commercially available e.g. from Invitrogen, Stemcell Technologies, Cellpro, Advanced Magnetics, or Miltenyi Biotec.), and depletion of specific cell types by methods such as eradication (e.g. killing) with specific antibodies or by affinity based purification based on negative selection (using e.g. magnetic cell separation techniques, FACS sorter and/or capture ELISA labeling). Such methods are described for example in THE HANDBOOK OF EXPERIMENTAL IMMUNOLOGY, Volumes 1 to 4, (D. N. Weir, editor) and FLOW CYTOMETRY AND CELL SORTING (A. Radbruch, editor, Springer Verlag, 2000).

According to other specific embodiments, the method is effected on immune cells not subjected to T cell isolation prior to adding the non-cellular agent capable of binding 4-1BB and activating 4-1BB signaling pathway and/or culturing in the presence of same.

Thus, according to specific embodiments, the method is effected without isolation of T cells from the immune cells prior to adding the non-cellular agent capable of binding 4-1BB and activating 4-1BB signaling pathway.

According to specific embodiments, the method is effected without isolation of T cells from the immune cells prior to culturing in the presence of the non-cellular agent capable of binding 4-1BB and activating 4-1BB signaling pathway.

According to specific embodiments, 4-1BB positive cells (e.g. T cells) are not isolated from the immune cells at any stage of the methods disclosed herein.

Thus, according to specific embodiments, the method is effected on immune cells not subjected to isolation of 4-1BB positive cells prior to adding the non-cellular agent capable of binding 4-1BB and activating 4-1BB signaling pathway and/or culturing in the presence of same.

According to specific embodiments, the method is effected without isolation of 4-1BB positive cells from the immune cells prior to adding the non-cellular agent capable of binding 4-1BB and activating 4-1BB signaling pathway.

According to specific embodiments, the method is effected without isolation of 4-1BB positive cells from the immune cells prior to culturing in the presence of the non-cellular agent capable of binding 4-1BB and activating 4-1BB signaling pathway.

An alternative method to increase the percentage of specific immune cell types comprising T cells is to culture the immune cells under conditions that allow enrichment of the desired cell types.

Hence, according to specific embodiments, the method comprises pre-culturing the immune cells prior to addition of the non-cellular agent capable of binding 4-1BB and activating 4-1BB signaling pathway.

According to specific embodiments, the method is effected without isolation of T cells from the immune cells prior to the pre-culturing.

According to specific embodiments, the method is effected without isolation of 4-1BB positive cells from the immune cells prior to the pre-culturing.

According to specific embodiments the pre-culturing is effected for up to three weeks, up to two weeks or up to 1 week.

According to specific embodiments, the pre-culturing is effected for 1-14, 2-14, 5-14, 7-14, or 8-12 days.

According to specific embodiments, the pre-culturing is effected for at least 2 days.

According to specific embodiments, the pre-culturing is effected for 2-14, 2-13, 2-12, 2-11, 2-10, 2-9, 2-8 or 2-7 days.

According to specific embodiments, the pre-culturing is effected for 2-14 days.

According to specific embodiments, the pre-culturing is effected for at least 3 days.

According to specific embodiments, the pre-culturing is effected for 3-14, 3-13, 3-12, 3-11, 3-10, 3-9, 3-8 or 3-7 days.

According to specific embodiments, the pre-culturing is effected for 3-14 days.

According to specific embodiments, the pre-culturing is effected for 7-14 days.

According to specific embodiments, the pre-culturing is effected for 3-10 days.

According to specific embodiments, the pre-culturing is effected about 10 days.

According to specific embodiments, the pre-culturing is effected until enrichment of T cells in the immune cells is achieved.

Thus, according to specific embodiments, the pre-culturing is effected until the percentage of T cells in the immune cells is at least 70%, at least 80%, at least 85%, at least 90%, at least 95%.

According to a specific embodiment, the pre-culturing is effected until the percentage of T cells in the immune cells is at least 80%.

As used herein, the term “culturing”, “pre-culturing” or “culture” refers to at least immune cells comprising T cells and medium in an ex-vivo, in-vitro environment.

The culture is maintained under conditions necessary to support growth and survival, for example, an appropriate temperature (e.g., 37° C.) atmosphere (e.g., air plus 5% CO₂) and medium.

The culture may be in a glass, plastic or metal vessel that can provide an aseptic environment for cell culturing. According to specific embodiments, the culture vessel includes dishes, plates, flasks, bottles, vials, bags, bioreactors or any device that can be used to grow cells.

Selection of the medium is well within the capabilities of skilled in the art. Thus, for example, the medium used can be a water-based medium which includes a combination of substances such as salts, nutrients, minerals, vitamins, amino acids, nucleic acids and/or proteins such as cytokines, growth factors and hormones, all of which are needed for cell proliferation and survival. Non-limiting examples of specific mediums include serum-free dendritic cell medium (can be obtained from examples from CellGernix); or RPMI or DMEM (can be obtained for example from Sigma-Aldrich or Biological Industries, Beit Haemek, Israel), supplemented with the necessary additives. For Example, the medium may be supplemented with L-glutamine, non-essential amino acids, sodium pyruvate, antibiotic/antimycotic solution, 2-mercaptoethanol and serum.

According to specific embodiments, the medium is supplemented with a cytokine.

Thus, according to specific embodiments, the method comprises adding at least one cytokine to the immune cells.

According to specific embodiments, pre-culturing of the immune cells prior to addition of the non-cellular agent capable of binding 4-1BB and activating 4-1BB signaling pathway is effected in the presence of at least one exogenous cytokine.

According to specific embodiments, culturing of the immune cells with the non-cellular agent capable of binding 4-1BB and activating 4-1BB signaling pathway is effected in the presence of at least one exogenous cytokine.

According to specific embodiments, the cytokine is capable of inducing survival, activation and/or proliferation of a T cell.

According to specific embodiments, the cytokine preferably induces survival and growth of T cells thereby enriching the population of T cells in the immune cells obtained from the subject.

Non-limiting examples of cytokines that can be used according to specific embodiments of the present invention include IL-2, IL-6, IL-4, IL-12, IL-7, IFNα, IL-12, IFN-gamma, TNF-α, IL-15, IL-1, IL-21 and GM-CSF.

According to specific embodiments, the cytokine is selected from the group consisting of IL-2, IL-15, IL-21, IL12 and 1L-7.

According to specific embodiments, the cytokine is cytokine comprises IL-2, IL-15 and IL-21.

Preferably, all ingredients included in the culture medium of the present invention are substantially pure, with a tissue culture grade.

According to some embodiments of the invention, the culture medium is devoid of any animal contaminants, i.e., animal cells, fluid or pathogens (e.g., viruses infecting animal cells), i.e., being xeno-free.

It should be noted that the culture medium may be periodically refreshed to maintain sufficient levels of supplements and to remove metabolic waste products that can damage the cells.

As noted, the method comprises adding to the immune cells a non-cellular agent capable of binding 4-1BB and activating said 4-1BB signaling pathway and culturing the immune cells in the presence of same.

As used herein, “adding” refers to adding to the culture/composition a substance which was not present in the immune cells sample. Also referred to as an exogenous agent.

As used herein, an “agent capable of binding 4-1BB and activating said 4-1BB signaling pathway” refers to a non-cellular (i.e., cell-free) substance that can bind 4-1BB which is expressed on a T cell and thereby transmit a co-stimulatory signal resulting in activation of the T cell.

As used herein the term “4-1BB (also known as CD137 and TNFRSF9)” refers to the polypeptide of the TNFRSF9 gene (Gene ID 3604). According to a specific embodiment, the 4-1BB protein refers to the human protein, such as provided in the following GenBank Number NP_001552.

Assays for testing binding are well known in the art and include, but not limited to flow cytometry, Biacore, bio-layer interferometry Blitz@ assay, HPLC.

According to specific embodiments, the agent binds 4-1BB with a Kd>10⁻⁶ M, >10⁻⁷ M, >10⁻⁸ M or >10⁻⁹ M, each possibility represents a separate embodiment of the present invention.

According to specific embodiments, the agent binds 4-1BB with a Kd of about 0.1-1000 nM, 0.1-100 nM or 1-100 nM, each possibility represents a separate embodiment of the claimed invention.

As used herein, the phrase “co-stimulatory signal” refers to transmission of a secondary antigen independent stimulatory signal (e.g. 4-1BB signal) resulting in activation of the immune cell (e.g. T cell expressing 4-1BB).

As used herein the terms “activating” or “activation” refer to the process of stimulating an immune cell (e.g. T cell) that results in cellular proliferation, maturation, cytokine production and/or induction of regulatory or effector functions.

Methods of determining signaling of a 4-1BB pathway are well known in the art, and include, but are not limited to, binding assay using e.g. Biacore, HPLC or flow cytometry, enzymatic activity assays such as kinase activity assays, and expression of molecules involved in the signaling cascade using e.g. PCR, Western blot, immunoprecipitation and immunohistochemistry. Additionally or alternatively, determining transmission of a 4-1BB signal can be effected by evaluating immune cell activation or function. Methods of evaluating immune cell activation or function are well known in the art and include, but are not limited to, proliferation assays such as CFSE staining, MTS, Alamar blue, BRDU and thymidine incorporation, cytotoxicity assays such as CFSE staining, chromium release, Calcin AM, cytokine (e.g. IL-8) secretion assays such as intracellular cytokine staining, ELISPOT and ELISA, expression of activation markers such as CD25, CD69, CD137, CD107a, PD1, and CD62L using flow cytometry.

According to specific embodiments, determining the activation of 4-1BB signaling pathway is effected in-vitro or ex-vivo e.g. in a mixed lymphocyte reaction (MLR).

For the same culture conditions the activation of 4-1BB signaling pathway is generally expressed in comparison to the signaling in a cell of the same species but not contacted with the non-cellular agent; or contacted with a vehicle control, also referred to as control.

Non-limiting examples of non-cellular agents capable of binding 4-1BB and activating said 4-1BB signaling pathway are described in details hereinbelow.

According to specific embodiments, the non-cellular agent is also capable of binding a ligand or receptor of a type I membrane protein.

Additionally or alternatively, the method further comprises adding to the immune cells a non-cellular agent capable of binding a ligand or receptor of a type I membrane protein and culturing the immune cells in the presence of same.

As used herein, the phrase “type I membrane protein” refers to a transmembrane protein having an N-terminus extracellular domain.

Non-limiting examples of such Type I membrane proteins include PD1, SIRPα, LAG3, BTN3A1, CD27, CD80, CD86, ENG, NLGN4X, CD84, TIGIT, CD40, IL-8, IL-10, CD164, LY6G6F, CD28, CTLA4, BTLA, LILRB1/2/3/4, TYROBP, ICOS, VEGFA, CSF1, CSF1R, VEGFB, BMP2, BMP3, GDNF, PDGFC, PDGFD, RAET1E, CD155, CD166, MICA, NRG1, HVEM, DR3, TEK, TGFB1, LY96, CD96, KIT, CD244, and GFER.

According to specific embodiments, the Type I membrane protein is an immune modulator.

As used herein the term “immune modulator” refers to a protein that modulates an immune cell response (i.e. activation or function). Immune modulators can positively regulate immune cell activation or function or negatively regulate immune cell activation or function. Such immune modulators are known in the art and include an immune-check point protein, a cytokine and the like.

According to specific embodiments, the immune modulator is an immune activator.

According to other specific embodiments, the immune modulator is an immune suppressor or inhibitor.

Non-limiting examples of Type I membrane protein immune modulators include, but are not limited to PD1, SIRPα, CD28, CSF1R, IL-8, IL-10, CTLA4, ICOS, CD27, CD80, CD86, TIGIT and LILRB1/2/3/4.

According to specific embodiments, the type I membrane protein is selected from the group consisting of PD1 and SIRPα.

According to specific embodiments, the type I membrane protein is PD1.

According to specific embodiments, the type I membrane protein is SIRPα. Assays for testing binding are well known in the art and are further described hereinabove.

According to specific embodiments, the agent binds a ligand or receptor of a type I membrane protein with a Kd>10⁻⁶ M, >10⁻⁷ M, >10⁻⁸ M or >10⁻⁹ M, each possibility represents a separate embodiment of the present invention.

According to specific embodiments, the agent binds a ligand or receptor of a type I membrane protein with a Kd of about 1 nM-100 μM, 10-nM-10 μM, 100 nM-100 μM, 200 nM-10 μM, each possibility represents a separate embodiment of the claimed invention.

According to specific embodiments, the agent binds a ligand or receptor of a type I membrane protein with a Kd of about 0.1-100 μM, 0.1-10 μM, 1-10 μM, 0.1-5 μM, or 1-2 μM, each possibility represents a separate embodiment of the present invention.

Non-limiting examples of non-cellular agents capable of binding a ligand or receptor of a type I membrane protein are described in details hereinbelow.

According to specific embodiments, the non-cellular agent is an antibody, a polypeptide or a small molecule.

According to specific embodiments, the non-cellular agent is an antibody.

According to specific embodiments the antibody is capable of specifically binding 4-1BB.

According to specific embodiments, the antibody specifically binds at least one epitope of 4-1BB.

According to specific embodiments the antibody is capable of specifically binding a ligand or a receptor of a type I membrane protein (e.g. PDL1, CD47).

According to specific embodiments, the antibody specifically binds at least one epitope of a ligand or a receptor of a type I membrane protein (e.g. PDL1, CD47).

As used herein, the term “epitope” refers to any antigenic determinant on an antigen to which the paratope of an antibody binds. Epitopic determinants usually consist of chemically active surface groupings of molecules such as amino acids or carbohydrate side chains and usually have specific three dimensional structural characteristics, as well as specific charge characteristics.

The term “antibody” as used in this invention includes intact molecules as well as functional fragments thereof, such as Fab, F(ab′)2, Fv, scFv, dsFv, or single domain molecules such as VH and VL that are capable of binding to an epitope of an antigen.

The antibody may be mono-specific (capable of recognizing one epitope or protein), bi-specific (capable of binding two epitopes or proteins) or multi-specific (capable of recognizing multiple epitopes or proteins).

Thus, according to specific embodiments, the antibody is a bi-specific antibody binding 4-1BB and a ligand or a receptor of a type I membrane protein (e.g. a 4-1BB-PDL1 antibody or a 4-1BB-CD47 antibody).

Suitable antibody fragments for practicing some embodiments of the invention include a complementarity-determining region (CDR) of an immunoglobulin light chain (referred to herein as “light chain”), a complementarity-determining region of an immunoglobulin heavy chain (referred to herein as “heavy chain”), a variable region of a light chain, a variable region of a heavy chain, a light chain, a heavy chain, an Fd fragment, and antibody fragments comprising essentially whole variable regions of both light and heavy chains such as an Fv, a single chain Fv Fv (scFv), a disulfide-stabilized Fv (dsFv), a single domain antibody (nanobody), an Fab, an Fab′, and an F(ab′)2.

The antibody may be monoclonal or polyclonal.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference).

Selection of the antibody used is well within the capability of those skilled in the art.

Non-limiting examples of anti-4-1BB antibodies that can be used with specific embodiments include Urelumab (BMS-663513), Utomilumab (PF-05082566), GEN 1042 [BioNTech SE (Genmab)], LOAd703 (Lokon Pharma), ADG-106 (Adagene), PRS-343 (Pieris Pharmaceuticals), CTX-471 (Compass Therapeutics LLC), INBRX-105 (Elpiscience; Inhibrx), LVGN6051 (Lyvgen Biopharma Ltd), MCLA-145 (Merus NV), MP-0310 (Molecular Partners), ATOR-1017 (Alligator Bioscience AB), RG-6076 (Roche), RG-7827 (Roche), AGEN-2373 (Agenus) and the antibodies disclosed in Xinyue Qi et al. (2019) Nature Communications volume 10, Article number: 2141; Marta Compte et al. (2018) Nature Communications volume 9, Article number: 4809; and International Patent Application Publication No. WO2003049755, the contents of which are fully incorporated herein by reference.

Non-limiting examples of anti-PDL1 antibodies that can be used with specific embodiments include Atezolisumab (Genentech), Avelumab (Merck Serono and Pfizer), Durvalumab (AstraZebeca), KN035 (3D Medicines) and Cosibelimab (Checkpoint Therapeutics).

Non-limiting examples of anti-CD47 antibodies that can be used with specific embodiments include magrolimab (Forty Seven), AO-176 (Arch Oncology), IBI-188 (Innovent Biologics), CC-90002 (Celgene), SRF-231 (Surface Oncology).

Non-limiting examples of anti-4-1BB-PDL1 bi-specific antibodies that can be used with specific embodiments include INBRX-105 (Inhibrx Inc), MCLA-145 (Merus NV), ABL-503 (TRIGR Therapeutics Inc), FS-222 (F-star Biotechnology Ltd), ND021 (Numab Innovation AG), PRS-344 (Pieris Pharmaceuticals Inc), ES101 (Elpiscience) and DuoBody®-PD-Llx4-1BB (Biontech and Genmab).

According to specific embodiments, the non-cellular agent is the naturally occurring activator of 4-1BB or a functional derivative or variant thereof which retains the ability to specifically bind 4-1BB and activates 4-1BB signaling.

Thus, according to specific embodiments, the non-cellular agent is a polypeptide comprising an amino acid sequence capable of binding 4-1BB and activating 4-1BB signaling pathway.

As used herein, the term “polypeptide” or “peptide” encompasses native peptides (either degradation products, synthetically synthesized peptides or recombinant peptides) and peptidomimetics (typically, synthetically synthesized peptides), as well as peptoids and semipeptoids which are peptide analogs, which may have, for example, modifications rendering the peptides more stable while in a body or more capable of penetrating into cells.

The term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids.

When referring to “an amino acid sequence” the meaning is to the chemical embodiment of the term and not the literal embodiment of the term.

As the polypeptide of some embodiments of the present invention is a natively occurring protein, the polypeptides of some embodiments of the invention may be purified.

Alternatively or additionally, the polypeptides of some embodiments of the invention may be synthesized by any techniques that are known to those skilled in the art of peptide synthesis, such as, but not limited to, solid phase and recombinant techniques.

For solid phase peptide synthesis, a summary of the many techniques may be found in J. M.

Stewart and J. D. Young, Solid Phase Peptide Synthesis, W. H. Freeman Co. (San Francisco), 1963 and J. Meienhofer, Hormonal Proteins and Peptides, vol. 2, p. 46, Academic Press (New York), 1973. For classical solution synthesis see G. Schroder and K. Lupke, The Peptides, vol. 1, Academic Press (New York), 1965.

Large scale peptide synthesis is described by Andersson Biopolymers 2000; 55(3):227-50.

According to specific embodiments, the polypeptide comprises an amino acid sequence of 4-1BBL.

As used herein the term “4-1BBL (also known as CD137L and TNFSF9)” refers to the polypeptide of the TNFSF9 gene (Gene ID 8744) or a functional homolog e.g., functional fragment thereof. According to specific embodiments, the term “4-1BBL” refers to a functional homolog of 4-1BBL polypeptide. According to specific embodiments, 4-1BBL is human 4-1BBL. According to a specific embodiment, the 4-1BBL protein refers to the human protein, such as provided in the following GenBank Number NP_003802.

According to specific embodiments, 4-1BBL amino acid sequence comprises SEQ ID NO: 1.

According to specific embodiments, 4-1BBL amino acid sequence consists of SEQ ID NO: 1.

As use herein, the phrase “functional homolog of a polypeptide of the TNFSF9 gene” or “functional fragment of a polypeptide of the TNFSF9 gene” refers to a portion of the polypeptide which maintains at least binding and activating 4-1BB signaling pathway activities of the full length 4-1BBL. According to specific embodiments, the functional homolog of fragment of the polypeptide of the TNFSF9 gene also maintains the ability of the full length 4-1BBL of forming a homotrimer.

Assays for testing binding and activating 4-1BB signaling pathway are well known in the art and are further described hereinabove.

According to specific embodiments, the 4-1BBL binds 4-1BB with a Kd of about 0.1-1000 nM, 0.1-100 nM, 1-100 nM, or 55 nM as determined by SPR, each possibility represents a separate embodiment of the claimed invention.

Methods of determining trimerization are well known in the art and include, but are not limited to NATIVE-PAGE, SEC-HPLC 2D gels, gel filtration, SEC-MALS, Analytical ultracentrifugation (AUC) Mass spectrometry (MS), capillary gel electrophoresis (CGE).

According to specific embodiments, the 4-1BBL comprises an extracellular domain of said 4-1BBL or a functional fragment thereof.

According to specific embodiments, 4-1BBL amino acid sequence comprises SEQ ID NO: 2.

According to specific embodiments, 4-1BBL amino acid sequence consists of SEQ ID NO: 2.

The term “4-1BBL” also encompasses functional homologues (naturally occurring or synthetically/recombinantly produced), which exhibit the desired activity (as defined hereinabove). Such homologues can be, for example, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical or homologous to SEQ ID NO: 1 or 2 (as further described hereinbelow).

As used herein, “identity” or “sequence identity” refers to global identity, i.e., an identity over the entire amino acid or nucleic acid sequences disclosed herein and not over portions thereof.

Sequence identity or homology can be determined using any protein or nucleic acid sequence alignment algorithm such as Blast, ClustalW, and MUSCLE.

The homolog may also refer to an ortholog, a deletion, insertion, or substitution variant, including an amino acid substitution, as further described hereinbelow.

According to specific embodiments, the 4-1BBL polypeptide may comprise conservative and/or non-conservative amino acid substitutions.

The term “conservative substitution” as used herein, refers to the replacement of an amino acid present in the native sequence in the peptide with a naturally or non-naturally occurring amino or a peptidomimetics having similar steric properties. Where the side-chain of the native amino acid to be replaced is either polar or hydrophobic, the conservative substitution should be with a naturally occurring amino acid, a non-naturally occurring amino acid or with a peptidomimetic moiety which is also polar or hydrophobic (in addition to having the same steric properties as the side-chain of the replaced amino acid).

As naturally occurring amino acids are typically grouped according to their properties, conservative substitutions by naturally occurring amino acids can be easily determined bearing in mind the fact that in accordance with the invention replacement of charged amino acids by sterically similar non-charged amino acids are considered as conservative substitutions.

For producing conservative substitutions by non-naturally occurring amino acids it is also possible to use amino acid analogs (synthetic amino acids) well known in the art. A peptidomimetic of the naturally occurring amino acid is well documented in the literature known to the skilled practitioner.

When affecting conservative substitutions the substituting amino acid should have the same or a similar functional group in the side chain as the original amino acid.

The phrase “non-conservative substitutions” as used herein refers to replacement of the amino acid as present in the parent sequence by another naturally or non-naturally occurring amino acid, having different electrochemical and/or steric properties. Thus, the side chain of the substituting amino acid can be significantly larger (or smaller) than the side chain of the native amino acid being substituted and/or can have functional groups with significantly different electronic properties than the amino acid being substituted. Examples of non-conservative substitutions of this type include the substitution of phenylalanine or cycohexylmethyl glycine for alanine, isoleucine for glycine, or —NH—CH[(—CH₂)₅—COOH]—CO— for aspartic acid. Those non-conservative substitutions which fall under the scope of the present invention are those which still constitute a peptide having neuroprotective properties.

According to specific embodiments, the 4-1BBL amino acid sequence does not comprise the amino acid segment A1-V6, A1-G14 or A1-E23 corresponding to SEQ ID NO: 2.

According to specific embodiments, the 4-1BBL amino acid sequence does not comprise any of amino acid residues A1-V6 or A1-G14 or A1-E23 corresponding to SEQ ID NO: 2.

According to specific embodiments, the 4-1BBL amino acid sequence does not comprise the amino acid segment G198-E205 corresponding to SEQ ID NO: 2.

According to specific embodiments, the 4-1BBL amino acid sequence does not comprise any of amino acid residues G198-E205 corresponding to SEQ ID NO: 2.

As used herein, the phrase “corresponding to SEQ ID NO: 2” intends to include the corresponding amino acid residue relative to any other 4-1BBL amino acid sequence.

According to specific embodiments, 4-1BBL amino acid sequence comprises 100-254 amino acids, 150-250 amino acids, 100-250 amino acids, 150-220 amino acids, 180-220 amino acids, 180-210 amino acids, 185-205 amino acids, 185-200 amino acids, 185-199 amino acids, 170-197 amino acids, 170-182 amino acids, 190-210 amino acids, each possibility represents a separate embodiment of the present invention.

The 4-1BBL of some embodiments of the present invention is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical or homologous to SEQ ID NO: 2, 3, 4, 5, 6, 7, 8, 9, or 10, each possibility represents a separate embodiment of the present invention.

According to specific embodiments, the 4-1BBL amino acid sequence comprises an amino acid sequence selected from the group consisting of SEQ ID NO: 2-10.

According to specific embodiments, the 4-1BBL amino acid sequence consists of an amino acid sequence selected from the group consisting of SEQ ID NO: 2-10.

The 4-1BBL of some embodiments of the present invention is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical or homologous to SEQ ID NO: 3.

According to specific embodiments, the 4-1BBL amino acid sequence comprises SEQ ID NO: 3.

According to specific embodiments, the 4-1BBL amino acid sequence consists of SEQ ID NO: 3.

According to specific embodiments, the amino acid sequence of 4-1BBL comprises three repeats of a 4-1BBL amino acid sequence.

According to specific embodiments, the three repeats have an identical amino acid sequence.

According to other specific embodiments, the three repeats are distinct, i.e. have different amino acid sequences.

According to specific embodiments, the 4-1BBL amino acid sequence comprises three repeats of an amino acid sequence comprising SEQ ID NO: 3.

According to specific embodiments, the 4-1BBL amino acid sequence comprises three repeats of an amino acid sequence consisting of SEQ ID NO: 3.

According to specific embodiments, the amino acid sequence of 4-1BBL does not comprise a linker between each of the three repeats of the 4-1BBL amino acid sequence.

According to specific embodiments, the amino acid sequence of 4-1BBL comprises a linker between each of the three repeats of the 4-1BBL amino acid sequence.

Any linker known in the art can be used with specific embodiments of the invention.

According to specific embodiments, the linker may be derived from naturally-occurring multi-domain proteins or is an empirical linker as described, for example, in Chichili et al., (2013), Protein Sci. 22(2): 153-167, Chen et al, (2013), Adv Drug Deliv Rev. 65(10): 1357-1369, the entire contents of which are hereby incorporated by reference. In some embodiments, the linker may be designed using linker designing databases and computer programs such as those described in Chen et al., (2013), Adv Drug Deliv Rev. 65(10): 1357-1369 and Crasto et al., (2000), Protein Eng. 13(5):309-312, the entire contents of which are hereby incorporated by reference.

According to specific embodiments, the linker may be functional. For example, without limitation, the linker may function to improve the folding and/or stability, improve the expression, improve the pharmacokinetics, and/or improve the bioactivity of the polypeptide.

According to specific embodiments, the linker is a synthetic linker such as PEG.

According to specific embodiments, the linker is a polypeptide.

Non-limiting examples of polypeptide linkers include linkers having the sequence LE, GGGGS (SEQ ID NO: 11), (GGGGS)ₙ (n=1-4) (SEQ ID NO: 12), GGGGSGGGG (SEQ ID NO: 13), (GGGGS)×2 (SEQ ID NO: 14), (GGGGS)×2+GGGG (SEQ ID NO: 15), (Gly)₈, (Gly)₆, (EAAAK)ₙ (n=1-3) (SEQ ID NO: 16), A(EAAAK)ₙA (n=2-5) (SEQ ID NO: 17), AEAAAKEAAAKA (SEQ ID NO: 18), A(EAAAK)₄ALEA(EAAAK)₄A (SEQ ID NO: 19), PAPAP (SEQ ID NO: 20), KESGSVSSEQ LAQFRSLD (SEQ ID NO: 21), EGKSSGSGSESKST (SEQ ID NO: 22), GSAGSAAGSGEF (SEQ ID NO: 23), and (XP)ₙ, with X designating any amino acid, e.g., Ala, Lys, or Glu.

According to specific embodiments, the linker is at a length of one to six amino acids.

According to specific embodiments, the linker is substantially comprised of glycine and/or serine residues (e.g. about 30%, or about 40%, or about 50%, or about 60%, or about 70%, or about 80%, or about 90%, or about 95%, or about 97% or 100% glycines and serines).

According to specific embodiments, the linker is a single amino acid linker.

In some embodiments of the invention, the one amino acid is glycine.

Thus, according to specific embodiments, the 4-1BBL amino acid sequence comprises an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 24.

According to specific embodiments, the 4-1BBL amino acid sequence comprises SEQ ID NO: 24.

According to specific embodiments, the 4-1BBL amino acid sequence consists of SEQ ID NO: 24.

According to specific embodiments, the non-cellular agent is the naturally occurring type I membrane protein (e.g. PD-1, SIRPα) or a functional derivative or variant thereof which retains the ability to specifically bind its ligand or receptor.

Thus, according to specific embodiments, the non-cellular agent is a polypeptide comprising an amino acid sequence of a type I membrane protein.

As used herein, the phrase “an amino acid sequence of a type I membrane protein” refers to a contiguous amino acids sequence of a type I membrane protein capable of at least binding the native ligand or receptor of the type I membrane protein.

According to specific embodiments, such an amino acid sequence comprises an extracellular domain of the type I membrane protein or a functional fragment thereof.

According to specific embodiments, the polypeptide comprises an amino acid sequence of PD1.

As used herein the term “PD1 (Programmed Death 1, also known as CD279)” refers to the polypeptide of the PDCD1 gene (Gene ID 5133) or a functional homolog e.g., functional fragment thereof. According to specific embodiments, the term “PD1” refers to a functional homolog of PD1 polypeptide. According to specific embodiments, PD1 is human PD1. According to a specific embodiment, the PD1 protein refers to the human protein, such as provided in the following GenBank Number NP_005009.

Two ligands for PD-1 have been identified so far, PDL1 and PDL2 (also known as B7-DC). According to a specific embodiment, the PDL1 protein refers to the human protein, such as provided in the following GenBank Number NP_001254635 and NP_054862. According to a specific embodiment, the PDL2 protein refers to the human protein, such as provided in the following GenBank Number NP_079515.

According to specific embodiments, PD1 amino acid sequence comprises SEQ ID NO: 25.

According to specific embodiments, PD1 amino acid sequence consists of SEQ ID NO: 25.

As use herein, the phrase “a functional homolog of the polypeptide of the PDCD1 gene” or “a functional fragment of the polypeptide of the PDCD1 gene” refers to a portion of the polypeptide which maintains the activity of the full length PD1 e.g., PD-L1 binding.

Assays for testing binding are well known in the art and include, but not limited to flow cytometry, Biacore, bio-layer interferometry Blitz® assay, HPLC.

According to specific embodiments, the PD1 binds PD-L1 with a Kd of 1 nM-100 PM, 10-nM-10 μM, 100 nM-100 μM, 200 nM-10 μM, as determined by SPR analysis, each possibility represents a separate embodiment of the present invention.

According to specific embodiments, the PD1 binds PDL1 with a Kd of about 270 nM as determined by SPR analysis.

According to specific embodiments, the PD1 binds PDL1 with a Kd of about 8-9 μM as determined by SPR analysis.

According to specific embodiments, the PD1 comprises an extracellular domain of said PD1 or a functional fragment thereof.

According to specific embodiments, PD1 amino acid sequence comprises SEQ ID NO: 26, 27 or 28.

According to specific embodiments, PD1 amino acid sequence consists of SEQ ID NO: 26, 27 or 28.

The term “PD1” also encompasses functional homologues (naturally occurring or synthetically/recombinantly produced), which exhibit the desired activity (i.e., binding PD-L1 and/or PD-L2). Such homologues can be, for example, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical or homologous to the polypeptide SEQ ID NO: 25, 26, 27 or 28 (as further described hereinbelow).

According to specific embodiments, the PD1 polypeptide may comprise conservative and/or non-conservative amino acid substitutions. Such substitution are known in the art and disclosed e.g. in Maute et al. PNAS, 2015 Nov. 24; 112(47): E6506-14; Ju Yeon et al. Nature Communications 2016 volume 7, Article number: 13354 (DOI: 10.1038/ncomms13354); and Zack K M et al. Structure. 2015 23(12): 2341-2348 (DOI:10.1016/j.str.2015.09.010), the contents of which are fully incorporated herein by reference.

According to specific embodiments, one or more amino acid mutations are located at an amino acid residue selected from: V39, L40, N41, Y43, R44, M45, S48, N49, Q50, T51, D52, K53, A56, Q63, G65, Q66, V72, H82, M83, R90, Y96, L97, A100, 5102, L103, A104, P105, K106, and A107 corresponding to the PD1 amino acid sequence set forth in SEQ ID NO: 27.

According to specific embodiments, one or more amino acid mutations are located at an amino acid residue selected from: V39, L40, N41, Y43, R44, M45, S48, N49, Q50, T51, D52, K53, A56, Q63, G65, Q66, C68, V72, H82, M83, R90, Y96, L97, A100, 5102, L103, A104, P105, K106, and A107 corresponding to the PD1 amino acid sequence set forth in SEQ ID NO: 27.

According to specific embodiments, one or more amino acid changes are selected from the group consisting of: (1) V39H or V39R; (2) L40V or L40I; (3) N41I or N41V; (4) Y43F or Y43H; (5) R44Y or R44L; (6) M45Q, M45E, M45L, or M45D; (7) S48D, S48L, S48N, S48G, or S48V; (8) N49C, N49G, N49Y, or N49S; (9) Q50K, Q50E, or Q50H; (10) T51V, T51L, or T51A; (11) D52F, D52R, D52Y, or D52V; (12) K53T or K53L; (13) A56S or A56L; (14) Q63T, Q63I, Q63E, Q63L, or Q63P; (15) G65N, G65R, G65I, G65L, G65F, or G65V; (16) Q66P; (17) V72I; (18) H82Q; (19) M83L or M83F; (20) R90K; (21) Y96F; (22) L97Y, L97V, or L97I; (23) A100I or A100V; (24) S102T or S102A; (25) L103I, L103Y, or L103F; (26) A104S, A104H, or A104D; (27) P105A; (28) K106G, K106E, K106I, K106V, K106R, or K106T; and (29) A107P, A107I, or A107V corresponding to the PD1 amino acid sequence set forth in SEQ ID NO: 27.

According to specific embodiments, one or more amino acid changes are selected from the group consisting of: (1) V39H or V39R; (2) L40V or L40I; (3) N41I or N41V; (4) Y43F or Y43H; (5) R44Y or R44L; (6) M45Q, M45E, M45L, or M45D; (7) S48D, S48L, S48N, S48G, or S48V; (8) N49C, N49G, N49Y, or N49S; (9) Q50K, Q50E, or Q50H; (10) T51V, T51L, or T51A; (11) D52F, D52R, D52Y, or D52V; (12) K53T or K53L; (13) A56S or A56L; (14) Q63T, Q63I, Q63E, Q63L, or Q63P; (15) G65N, G65R, G65I, G65L, G65F, or G65V; (16) Q66P; (17) C68S (18), V72I; (19) H82Q; (20) M83L or M83F; (21) R90K; (22) Y96F; (23) L97Y, L97V, or L97I; (24) A100I or A100V; (25) S102T or S102A; (26) L103I, L103Y, or L103F; (27) A104S, A104H, or A104D; (28) P105A; (29) K106G, K106E, K106I, K106V, K106R, or K106T; and (30) A107P, A107I, or A107V corresponding to the PD1 amino acid sequence set forth in SEQ ID NO: 27.

According to specific embodiments, an amino acid mutation is located at an amino acid residue C93 corresponding to the PD1 amino acid sequence set forth in SEQ ID NO: 25 (e.g. equivalent to an amino acid residue C68 corresponding to the PD1 amino acid sequence set forth in SEQ ID NO: 27).

According to specific embodiments, the PD1 polypeptide may comprise a C to S amino acid modification in a position corresponding to amino acid residue 93 of the PD1 amino acid sequence set forth in SEQ ID NO: 25 (e.g. equivalent to amino acid residue 68 of the PD1 amino acid sequence set forth in SEQ ID NO: 27).

Thus, according to specific embodiments, the PD1 amino acid sequence comprises SEQ ID NO: 29 or 30.

According to specific embodiments, PD1 amino acid sequence consists of SEQ ID NO: 29 or 30.

As used herein, “corresponding to PD1 amino acid sequence as set forth in SEQ ID NO: 25 or 27” or “corresponding to SEQ ID NO: 25 or 27”, intends to include the corresponding amino acid residue relative to any other PD1 amino acid sequence.

The PD1 of some embodiments of the present invention is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical or homologous to the polypeptide SEQ ID NO: 26, 27, 28, 29, 30, 31, 32, 34, 35, 36, 37, 38, 39, 40, 41, 42, 43, 44, 45, 46, 47 or 48, each possibility represents a separate embodiment of the present invention.

According to specific embodiments, the PD1 amino acid sequence does not comprise any of amino acid segments P1-L5 and/or F146-V150 corresponding to SEQ ID NO: 28.

According to specific embodiments, the PD1 amino acid sequence does not comprise any of amino acid residues P1-L5 and/or F146-V150 corresponding to SEQ ID NO: 28.

According to specific embodiments, PD1 amino acid sequence comprises 100-288 amino acids, 100-200 amino acids, 120-180 amino acids, 120-160, 130-170 amino acids, 130-160, 130-150, 140-160 amino acids, 145-155 amino acids, 123-166 amino acids, 138-145 amino acids, 123-148 amino acids, 126-148 amino acids, 123-140 amino acids, 126-140 amino acids, 127-140 amino acids, 130-140 amino acids, each possibility represents a separate embodiment of the present invention.

According to specific embodiments, the PD1 amino acid sequence comprises an amino acid sequence selected from the group consisting of SEQ ID NO: 26-48.

According to specific embodiments, the PD1 amino acid sequence consists of an amino acid sequence selected from the group consisting of SEQ ID NO: 26-48.

The PD1 of some embodiments of the present invention is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical or homologous to SEQ ID NO: 28.

According to specific embodiments, the PD1 amino acid sequence comprises SEQ ID NO: 28.

According to specific embodiments, the PD1 amino acid sequence consists of SEQ ID NO: 28.

According to specific embodiments, the polypeptide comprises an amino acid sequence of SIRPα.

As used herein the term “SIRPα (Signal Regulatory Protein Alpha, also known as CD172a)” refers to the polypeptide of the SIRPA gene (Gene ID 140885) or a functional homolog e.g., functional fragment thereof. According to specific embodiments, the term “SIRPα” refers to a functional homolog of SIRPα polypeptide. According to specific embodiments, SIRPα is human SIRPα. According to a specific embodiment, the SIRPα protein refers to the human protein, such as provided in the following GenBank Number NP_001035111, NP_001035112, NP_001317657 or NP_542970.

According to specific embodiments, SIRPα amino acid sequence comprises SEQ ID NO: 82.

According to specific embodiments, SIRPα amino acid sequence consists of SEQ ID NO: 82.

As use herein, the phrase “functional homolog of the polypeptide of the SIRPA gene” or “functional fragment of the polypeptide of the SIRP1 gene” refers to a portion of the polypeptide which maintains the activity of the full length SIRPα e.g., CD47 binding.

Assays for testing binding are well known in the art and are further described hereinabove and below.

According to a specific embodiment, the CD47 protein refers to the human protein, such as provided in the following GenBank Numbers NP_001768 or NP_942088.

According to specific embodiments, the SIRPα binds CD47 with a Kd of 0.1-100 μM, 0.1-10 μM, 1-10 μM, 0.1-5 μM, or 1-2 μM as determined by SPR, each possibility represents a separate embodiment of the present invention.

According to specific embodiments, the SIRPα comprises an extracellular domain of said SIRPα or a functional fragment thereof.

According to specific embodiments, SIRPα amino acid sequence comprises SEQ ID NO: 83.

According to specific embodiments, SIRPα amino acid sequence consists of SEQ ID NO: 83.

The term “SIRPα” also encompasses functional homologues (naturally occurring or synthetically/recombinantly produced), which exhibit the desired activity (i.e., binding CD47). Such homologues can be, for example, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical or homologous to SEQ ID NO: 82 or 83 (as further described hereinbelow).

According to specific embodiments, the SIRPα polypeptide may comprise conservative and/or non-conservative amino acid substitutions. Such substitutions are known in the art and disclosed e.g. in Weiskopf K et al. Science. (2013); 341(6141):88-91, the contents of which are fully incorporated herein by reference.

According to specific embodiments, one or more amino acid mutations are located at an amino acid residue selected from: L4, V6, A21, A27, I31, E47, K53, E54, H56, V63, L66, K68, V92 and F96 corresponding to the SIRPα amino acid sequence set forth in SEQ ID NO: 83.

According to specific embodiments, the SIRPα amino acid sequence comprises a mutation at an amino acid residue selected from the group consisting of L4, A27, E47 and V92 corresponding to the SIRPα amino acid sequence set forth in SEQ ID NO: 83.

According to specific embodiments, one or more amino acid mutations are selected from the group consisting of: L4V or L4I, V6I or V6L, A21V, A27I or A27L, 131F or 131T, E47V or E47L, K53R, E54Q, H56P or H56R, V63I, L66T or L66G, K68R, V92I and F94L or F94V corresponding to the SIRPα amino acid sequence set forth in SEQ ID NO: 83.

According to specific embodiments, the SIRPα amino acid sequence comprises a mutation selected from the group consisting of L4I, A27I, E47V and V92I corresponding to the SIRPα amino acid sequence set forth in SEQ ID NO: 83.

As used herein, the phrase “corresponding to the SIRPα amino acid sequence set forth in SEQ ID NO: 83” or “corresponding to SEQ ID NO: 83” intends to include the corresponding amino acid residue relative to any other SIRPα amino acid sequence.

According to specific embodiments, the SIRPα amino acid sequence comprises SEQ ID NO: 84.

According to specific embodiments, the SIRPα amino acid sequence consists of SEQ ID NO: 84.

The SIRP amino acid sequence of some embodiments of the present invention is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical or homologous to the polypeptide SEQ ID NO: 83, 84, 85 or 86, each possibility represents a separate embodiment of the present invention.

According to specific embodiments, the SIRPα amino acid sequence does not comprise the amino acid segment K117-Y343 corresponding to SEQ ID NO: 83.

According to specific embodiments, the SIRPα amino acid sequence does not comprise any of amino acid residues K117-Y343 corresponding to SEQ ID NO: 83.

According to specific embodiments, SIRPα amino acid sequence comprises 100-504, 100-500 amino acids, 150-450 amino acids, 200-400 amino acids, 250-400 amino acids, 300-400 amino acids, 320-420 amino acids, 340-350 amino acids, 300-400 amino acids, 340-450 amino acids, 100-200 amino acids, 100-150 amino acids, 100-125 amino acids, 100-120 amino acids, 100-119 amino acids, 105-119 amino acids, 110-119 amino acids, 115-119 amino acids, 105-118 amino acids, 110-118 amino acids, 115-118 amino acids, 105-117 amino acids, 110-117 amino acids, 115-117 amino acids, each possibility represents a separate embodiment of the present invention.

According to specific embodiments, the SIRPα amino acid sequence comprises an amino acid sequence selected from the group consisting of SEQ ID NO: 83-86.

The SIRPα of some embodiments of the present invention is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical or homologous to SEQ ID NO: 83.

According to specific embodiments, the SIRPα amino acid sequence comprises SEQ ID NO: 83.

According to specific embodiments, the SIRPα amino acid sequence consists of SEQ ID NO: 83. According to specific embodiments, the polypeptide is a fusion polypeptide.

As used herein, the term “fusion polypeptide” refers to an amino acid sequence having two or more parts which are not found together in a single amino acid sequence in nature.

The fusion polypeptide of some embodiments comprises an amino acid sequence capable of binding 4-1BB and activating 4-1BB signaling pathway (e.g. 4-1BBL) attached to (e.g., as a translational fusion) a heterologous amino acid sequence.

The fusion polypeptide of some embodiments comprises an amino acid sequence of a type I membrane protein attached to (e.g., as a translational fusion) a heterologous amino acid sequence.

As used herein, the term “heterologous” refers to an amino acid sequence which is not native to the recited amino acid sequence (e.g. an amino acid sequence capable of binding 4-1BB and activating 4-1BB signaling pathway) at least in localization or is completely absent from the native sequence of the recited amino acid sequence.

The fusion polypeptide may or may not comprise a linker between the two or more parts (e.g. the amino acid sequence capable of binding 4-1BB and activating 4-1BB signaling pathway and the heterologous amino acid sequence). Any linker known in the art can be used with specific embodiments of the invention. Non-limiting examples of linkers that can be used are further described hereinabove.

Thus, for example, the non-cellular agent can be an Fc-fusion polypeptide.

As used herein the term, “Fc-fusion polypeptide” refers to a polypeptide comprising an amino acid sequence capable of binding 4-1BB and activating 4-1BB signaling pathway (e.g. 4-1BBL) and/or a polypeptide comprising an amino acid sequence of a type I membrane protein (e.g. PD-1, SIRPα) combined with an Fc domain of an antibody.

According to specific embodiments, the Fc domain is of an IgG antibody.

Several Fc-fusion proteins that can be used with specific embodiments are commercially available from e.g. AB Biosciences or ACRO Biosystems, Sino Biological or PeproTech.

Non-limiting examples of SIRPα-Fc proteins that can be used with specific embodiments include ALX-148 (ALX Oncology), TTI-621 and TTI-622 (Trillium Therapeutics).

Another example of a fusion polypeptide that can be used with specific embodiments of the present invention is a fusion polypeptide comprising an amino acid sequence of 4-1BBL and an amino acid sequence of a type I membrane protein.

Thus, according to specific embodiments, the non-cellular agent that can be used with specific embodiments of the present invention is a fusion polypeptide comprising an amino acid sequence of 4-1BBL and an amino acid sequence of PD1 (also referred to herein as a PD1-4-1BBL fusion polypeptide).

According to specific embodiments, the PD1 is N-terminal to the 4-1BBL.

According to other specific embodiments, the PD1 is C-terminal to the 4-1BBL.

Thus, non-limiting examples of PD1-4-1BBL fusion polypeptide that can be used with specific embodiments of the present inventions are provided in SEQ ID NOs: 49-81.

According to specific embodiments, the PD1-4-1BBL fusion polypeptide of some embodiments of the present invention is comprises an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80 or 81, each possibility represents a separate embodiment of the present invention.

According to specific embodiments, the PD1-4-1BBL fusion polypeptide comprises an amino acid sequence having at least 80% identity to SEQ ID NO: 49.

According to specific embodiments, the PD1-4-1BBL fusion polypeptide comprises an amino acid sequence having at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 49.

According to specific embodiments, the PD1-4-1BBL fusion polypeptide comprises SEQ ID NO: 49.

According to specific embodiments, the PD1-4-1BBL fusion polypeptide is as set forth in SEQ ID NO: 49.

According to specific embodiments, the PD1-4-1BBL fusion polypeptide comprises an amino acid sequence having at least 80% identity to SEQ ID NO: 74.

According to specific embodiments, the PD1-4-1BBL fusion polypeptide comprises an amino acid sequence having at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 74.

According to specific embodiments, the PD1-4-1BBL fusion polypeptide comprises SEQ ID NO: 74.

According to specific embodiments, the PD1-4-1BBL fusion polypeptide is as set forth in SEQ ID NO: 74.

According to specific embodiments, the non-cellular agent that can be used with specific embodiments of the present invention is a fusion polypeptide comprising an amino acid sequence of 4-1BBL and an amino acid sequence of SIRPα (also referred to herein as a SIRPα-4-1BBL fusion polypeptide).

According to specific embodiments, the SIRPα is N-terminal to the 4-1BBL.

According to other specific embodiments, the SIRPα is C-terminal to the 4-1BBL.

Thus, non-limiting examples of SIRPα-4-1BBL fusion polypeptides that can be used with specific embodiments of the present inventions are provided in SEQ ID NOs: 87-101.

According to specific embodiments, the SIRPα-4-1BBL fusion polypeptide of some embodiments of the present invention is comprises an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NOs: 87, 88, 89, 90, 91, 92, 93, 94, 95, 96, 97, 98, 99, 100 or 101, each possibility represents a separate embodiment of the present invention.

According to specific embodiments, the SIRPα-4-1BBL fusion polypeptide comprises an amino acid sequence having at least 80% identity to SEQ ID NO: 89.

According to specific embodiments, the SIRPα-4-1BBL fusion polypeptide comprises an amino acid sequence having at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 89.

According to specific embodiments, the SIRPα-1BBL fusion polypeptide comprises SEQ ID NO: 89.

According to specific embodiments, the SIRPα-4-1BBL fusion polypeptide is as set forth in SEQ ID NO: 89.

According to specific embodiments, the non-cellular agent is a small molecule.

A non-limiting example of a small molecule capable of binding CD47 (i.e. a receptor of SIRPα) that can be used with specific embodiments is RRX-001 (EpicentRx).

According to specific embodiments, the non-cellular agent disclosed herein is soluble.

According to a specific embodiment, the non-cellular agent capable of binding 4-1BB and activating said 4-1BB signaling pathway is a soluble active 4-1BBL, such as described for examples in PMID: 29456671.

According to specific embodiments, the non-cellular agent disclosed herein is immobilized (i.e. to the culture vessel or to a solid support within the culture vessel e.g., insert).

According to specific embodiments, the non-cellular agent is capable of forming homo-trimers.

According to specific embodiments, the agent comprises a detectable tag. As used herein, in one embodiment the term “detectable tag” refers to any moiety that can be detected by a skilled practitioner using art known techniques. Detectable tags may be peptide sequences. Optionally the detectable tag may be removable by chemical agents or by enzymatic means, such as proteolysis. Detectable tags of some embodiments of the present invention can be used for purification of the agent. For example the term “detectable tag” includes chitin binding protein (CBP)-tag, maltose binding protein (MBP)-tag, glutathione-S-transferase (GST)-tag, poly(His)-tag, FLAG tag, Epitope tags, such as, V5-tag, c-myc-tag, and HA-tag, and fluorescence tags such as green fluorescent protein (GFP), red fluorescent protein (RFP), yellow fluorescent protein (YFP), blue fluorescent protein (BFP), and cyan fluorescent protein (CFP); as well as derivatives of these tags, or any tag known in the art. The term “detectable tag” also includes the term “detectable marker”.

Following addition of the non-cellular agent to the immune cells the cells are cultured in the presence of same.

According to specific embodiments, the method of culturing comprises repeated supplementation with the non-cellular agent i.e., the adding the non-cellular agent more than 1 time during the culturing period e.g., 2 times, 3 times 2-4 times.

According to specific embodiments, culturing with the non-cellular agent is effected for more than 7, more than 8, more than 9 more than 10, more than 11, more than 12 days.

According to specific embodiments, culturing with the non-cellular agent is effected for more than 10 days.

According to specific embodiments, culturing with the non-cellular agent is effected for about 14 days.

According to specific embodiments, culturing with the non-cellular agent is effected for 14 days.

According to specific embodiments, culturing with the non-cellular agent is effected for no longer that 21 days.

According to specific embodiments, culturing with the non-cellular agent is effected for no longer than 14 days.

According to specific embodiments, culturing with the non-cellular agent is effected for less than 15 days.

According to specific embodiments, culturing with the non-cellular agent is effected under conditions which allow enrichment of T cell clones reactive to cells associated with said pathology.

According to specific embodiments, culturing with the non-cellular agent is effected until at least 40%, at least 50%, at least 60%, at least 70%, at least 80%, at least 90% of the T cells are reactive to cells associated with said pathology.

According to specific embodiments, culturing with the non-cellular agent is effected under conditions which allow enrichment of T cells having a memory phenotype.

According to specific embodiments, culturing with the non-cellular agent is effected until at least 30%, at least 40%, at least 50%, at least 60%, at least 70%, at least 80%, at least 90% of the T cells are memory T cells.

Thus, according to specific embodiments, the method is effected on immune cells obtained from a subject that were not subjected to a culturing period with a T cell stimulatory agent capable of at least transmitting a primary activating signal to T cells.

According to specific embodiments, the pre-culturing is effected without adding to the immune cells a T cell stimulatory agent capable of at least transmitting a primary activating signal to T cells.

According to specific embodiments, the culturing is effected without adding to the immune cells a T cell stimulatory agent capable of at least transmitting a primary activating signal to T cells.

However, following the culturing with the non-cellular agent, the T cells may be further expanded.

Thus, according to specific embodiments, the method comprises expanding the T cells obtained by the method (e.g. following the culturing).

Expansion of T cells means proliferation, a growth in cell number throughout a culturing stage.

Typically, such an expansion is effected in the presence of a T cell stimulatory agent capable of at least transmitting a primary activating signal to T cells. Methods of determining the amount of the stimulatory agent and the ratio between the stimulatory agent and the T cells are well within the capabilities of the skilled in the art and thus are not specified herein.

As used herein the phrase, “T cell stimulatory agent” refers to an agent capable of at least transmitting a primary activating signal [e.g. ligation of the T-Cell Receptor (TCR) with the Major Histocompatibility Complex (MHC)/peptide complex on the Antigen Presenting Cell (APC)] resulting in cellular proliferation, maturation, cytokine production, phagocytosis and/or induction of regulatory or effector functions of the T cell. According to specific embodiments, the T cell stimulatory can also transmit a secondary co-stimulatory signal.

T cell stimulatory agents can activate the T cells in an antigen-dependent or -independent (i.e. polyclonal) manner.

According to specific embodiments, the T cell stimulatory agent comprises an antigen non-specific stimulator.

Non-specific stimulator are known to the skilled in the art. A non-limiting example of such non-specific stimulator is an agent capable of binding to a T cell surface structure and inducing the polyclonal stimulation of the T cell, such as but not limited to anti-CD3 antibody in combination with a co-stimulatory protein such as anti-CD28 antibody. Other non-limiting examples include anti-CD2, anti-CD137, anti-CD134, Notch-ligands, e.g. Delta-like 1/4, Jagged1/2 either alone or in various combinations with anti-CD3. Other agents that can induce polyclonal stimulation of T cells include, but not limited to mitogens, PHA, PMA-ionomycin, CEB and CytoStim (Miltenyi Biotech). According to specific embodiments, the antigen non-specific stimulator comprises anti-CD3 and anti-CD28 antibodies. According to specific embodiments, the T cell stimulator comprises anti-CD3 and anti-CD28 coated beads, such as the CD3CD28 MACSiBeads obtained from Miltenyi Biotec.

According to specific embodiments, the T cell stimulatory agent comprises an antigen-specific stimulator.

Non-limiting examples of antigen specific T cell stimulators include an antigen-loaded antigen presenting cell [APC, e.g. dendritic cell] and peptide loaded recombinant MHC. Thus, for example, a T cells stimulator can be a dendritic cell preloaded with a desired antigen (e.g. a tumor antigen) or transfected with mRNA coding for the desired antigen.

As used herein, the term “cancer antigen” refers to an antigen overexpressed or solely expressed by a cancerous cell as compared to a non-cancerous cell. A cancer antigen may be a known cancer antigen or a new specific antigen that develops in a cancer cell (i.e. neoantigens).

Non-limiting examples for known cancer antigens include MAGE-AI, MAGE-A2, MAGE-A3, MAGE-A4, MAGE-AS, MAGE-A6, MAGE-A7, MAGE-AS, MAGE-A9, MAGE-AIO, MAGE-A11, MAGE-A12, GAGE-I, GAGE-2, GAGE-3, GAGE-4, GAGE-5, GAGE-6, GAGE-7, GAGE-8, BAGE-1, RAGE-1, LB33/MUM-1, PRAME, NAG, MAGE-Xp2 (MAGE-B2), MAGE-Xp3 (MAGE-B3), MAGE-Xp4 (MAGE-B4), MAGE-C1/CT7, MAGE-C2, NY-ESO-1, LAGE-1, SSX-1, SSX-2(HOM-MEL-40), SSX-3, SSX-4, SSX-5, SCP-1 and XAGE, melanocyte differentiation antigens, p53, ras, CEA, MUCI, PMSA, PSA, tyrosinase, Melan-A, MART-I, gplOO, gp75, alphaactinin-4, Bcr-Abl fusion protein, Casp-8, beta-catenin, cdc27, cdk4, cdkn2a, coa-1, dek-can fusion protein, EF2, ETV6-AML1 fusion protein, LDLR-fucosyltransferaseAS fusion protein, HLA-A2, HLA-A11, hsp70-2, KIAA0205, Mart2, Mum-2, and 3, neo-PAP, myosin class I, OS-9, pml-RAR alpha fusion protein, PTPRK, K-ras, N-ras, Triosephosphate isomerase, GnTV, Herv-K-mel, NA-88, SP17, and TRP2-Int2, (MART-I), E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, plSOerbB-3, c-met, nm-23H1, PSA, TAG-72-4, CA 19-9, CA 72-4, CAM 17.1, NuMa, K-ras, alpha.-fetoprotein, 13HCG, BCA225, BTAA, CA 125, CA 15-3 (CA 27.29\BCAA), CA 195, CA 242, CA-50, CAM43, CD68\KP1, C0-029, FGF-5, 0250, Ga733 (EpCAM), HTgp-175, M344, MA-50, MG7-Ag, MOV18, NB\170K, NYCO-I, RCASI, SDCCAG16, TA-90 (Mac-2 binding protein\cyclophilin C-associated protein), TAAL6, TAG72, TLP, TPS, tyrosinase related proteins, TRP-1, or TRP-2.

Other tumor antigens that may be expressed are well-known in the art (see for example WO00/20581; Cancer Vaccines and Immunotherapy (2000) Eds Stern, Beverley and Carroll, Cambridge University Press, Cambridge). The sequences of these tumor antigens are readily available from public databases but are also found in WO 1992/020356 A1, WO 1994/005304 A1, WO 1994/023031 A1, WO 1995/020974 A1, WO 1995/023874 A1 & WO 1996/026214 A1.

Alternatively, or additionally, a tumor antigen may be identified using cancer cells obtained from the subject by e.g. biopsy.

Another non-limiting example of antigen specific T cell stimulator is a pathologic cell (e.g. cancer cell).

According to specific embodiments, the T cells obtained by the method are further isolated from the immune cells (e.g. following the culturing with the non-cellular agent and/or later on following the expanding). Methods of isolating T cells are known in the art and are further described hereinabove.

As the ex-vivo environment developed by the present inventors induced clonal selection of cancer-specific T cells, specific embodiments suggest such an ex-vivo environment as a platform for generating engineered T cells transduced with a T cell receptor.

Thus, according to specific embodiments, the method further comprises determining a sequence of a T cell receptor (TCR) expressed by at least one of the T cells obtained by the method (e.g. following their culturing with the non-cellular agent, or later on following their expansion or isolation).

Methods of determining a TCR sequence are known in the art, and include for example Sanger sequencing, high-throughput sequencing of genomic DNA e.g. ImmunoSEQ (Adaptive Biotechnologies).

Once a desired TCR has been sequenced and selected T cells can be transduced with a nucleic acid sequence encoding this TCR.

According to specific embodiments, the TCR sequence selected for transduction is at least 5% from the total TCR population with at least 10-fold higher abundancy compared to the same TCR in control immune cells not cultured with the non-cellular agent.

As used herein the phrase “transduced with a nucleic acid sequence encoding a TCR” or “transducing with a nucleic acid sequence encoding a TCR” refers to cloning of variable α- and β-chains from T cells with specificity against a desired antigen presented in the context of MHC. Methods of transducing with a TCR are known in the art and are disclosed e.g. in Nicholson et al. Adv Hematol. 2012; 2012:404081; Wang and Riviere Cancer Gene Ther. 2015 March; 22(2):85-94); and Lamers et al, Cancer Gene Therapy (2002) 9, 613-623.

Further, as the ex-vivo environment developed by the present inventors generated T cells with a memory phenotype, specific embodiments suggest such an ex-vivo environment as a platform for generating engineered CAR T cells with memory phenotype.

Thus, according to specific embodiments, the method further comprises transducing the T cells obtained by the method with a nucleic acid sequence encoding a chimeric antigen receptor (CAR) (e.g. following their culturing with the non-cellular agent, or later on following their expansion or isolation).

As used herein, the phrase “transduced with a nucleic acid sequence encoding a CAR” or “transducing with a nucleic acid sequence encoding a CAR” refers to cloning of a nucleic acid sequence encoding a chimeric antigen receptor (CAR), wherein the CAR comprises an antigen recognition moiety and a T-cell activation moiety. A chimeric antigen receptor (CAR) is an artificially constructed hybrid protein or polypeptide containing an antigen binding domain of an antibody (e.g., a single chain variable fragment (scFv)) linked to T-cell signaling or T-cell activation domains. Method of transducing with a CAR are known in the art and are disclosed e.g. in Davila et al. Oncoimmunology. 2012 Dec. 1; 1(9):1577-1583; Wang and Riviere Cancer Gene Ther. 2015 March; 22(2):85-94); Maus et al. Blood. 2014 Apr. 24; 123(17):2625-35; Porter D L. The New England journal of medicine. 2011, 365(8):725-733; Jackson H J, Nat Rev Clin Oncol. 2016; 13(6):370-383; and Globerson-Levin et al. Mol Ther. 2014; 22(5):1029-1038.

According to specific embodiments, the T cells are further expanded following the transducing.

Specific embodiments of the present invention also contemplates T cells and immune cell cultures obtainable according to the methods disclosed herein.

Thus, according to an aspect of the present invention, there is provided a cell culture comprising immune cells comprising T cells obtained from a subject having a pathology and an exogenous non-cellular agent capable of binding 4-1BB and activating 4-1BB signaling pathway, wherein the culture is more than 7 days old.

According to an alternative or additional aspect of the present invention, there is provided a cell culture comprising immune cells comprising T cells obtained from a subject having a pathology and an exogenous non-cellular agent capable of binding:

(i) 4-1BB and activating 4-1BB signaling pathway; and

(ii) a ligand or a receptor of a type I membrane protein,

wherein the culture is more than 7 days old.

According to an alternative or additional aspect of the present invention, there is provided a cell culture comprising immune cells comprising T cells obtained from a subject having a pathology, an exogenous non-cellular agent capable of binding 4-1BB and activating 4-1BB signaling pathway and an exogenous non-cellular agent capable of binding a ligand or a receptor of a type I membrane protein, wherein the culture is more than 7 days old.

According to an alternative or additional aspect of the present invention, there is provided a cell culture comprising immune cells comprising T cells obtained from a subject having a pathology and a non-cellular agent capable of binding 4-1BB and activating said 4-1BB signaling pathway in a level above the level obtained in the cell culture without addition of the non-cellular agent, wherein the culture is more than 7 days old.

According to an alternative or additional aspect of the present invention, there is provided a cell culture comprising immune cells comprising T cells obtained from a subject having a pathology and a non-cellular agent capable of binding:

(i) 4-1BB and activating said 4-1BB signaling pathway; and

(ii) a ligand or a receptor of a type I membrane protein,

in a level above the level obtained in the cell culture without addition of the non-cellular agent, wherein the culture is more than 7 days old.

According to an alternative or additional aspect of the present invention, there is provided a cell culture comprising immune cells comprising T cells obtained from a subject having a pathology, a non-cellular agent capable of binding 4-1BB and activating said 4-1BB signaling pathway and a non-cellular agent capable of binding a ligand or receptor of a type I membrane protein, wherein the culture is more than 7 days old and wherein the non-cellular agent is provided in a level above the level obtained in the cell culture without addition of the non-cellular agent.

According to specific embodiments, the culture is more than 8, more than 9 more than 10, more than 11, more than 12 days old.

According to specific embodiments, the culture is more than 10 days old.

According to specific embodiments, the culture is about 14 days old.

According to specific embodiments, the culture is 14 days old.

According to specific embodiments, the culture further comprises at least one cytokine in a level above the level obtained in the cell culture without addition of the at least one cytokine.

According to specific embodiments, the culture does not comprise an exogenous T cell stimulatory agent capable of at least transmitting a primary activating signal to said T cells.

According to specific embodiments, the culture is characterized by enrichment of T cell clones reactive to cells associated with the pathology.

According to specific embodiments, the culture comprises at least 50%, at least 60%, at least 70%, at least 80%, at least 90% T cells reactive to cells associated with said pathology.

According to specific embodiments, the culture is characterized by enrichment of T cells having a memory phenotype.

According to specific embodiments, the culture comprises at least 30%, at least 40%, at least 50%, at least 60%, at least 70%, at least 80%, at least 90% memory T cells.

According to an alternative or additional aspect of the present invention, there is provided a T cell obtainable by the method disclosed herein.

According to specific embodiments, the immune cells or T cells used and/or obtained according to the present invention can be freshly isolated or stored e.g., cryopreserved (i.e. frozen) at e.g. liquid nitrogen temperature at any stage for long periods of time (e.g., months, years) for future use.

Methods of cryopreservation are commonly known by one of ordinary skill in the art and are disclosed e.g. in International Patent Application Publication Nos. WO2007054160 and WO 2001039594 and US Patent Application Publication No. US20120149108.

According to specific embodiments, the cells obtained according to the present invention can be stored in a cell bank or a depository or storage facility.

Consequently, the present teachings further suggest the use of the immune cells or T cells and the methods of the present invention as, but not limited to, a source for adoptive immune cells therapies e.g. for diseases that can benefit from activating immune cells e.g. a hyper-proliferative disease; a disease associated with immune suppression and infections.

Thus, according to specific embodiments, the method comprises adoptively transferring the immune cells and/or the T cells obtainable by the method to a subject in need thereof.

According to an aspect of the present invention, there is provided the T cells obtainable by the method for use in adoptive cell therapy to a subject in need thereof.

According to an additional or an alternative aspect of the present invention, there is provided a method of treating a pathology that can benefit from adoptive T cell therapy in a subject in need thereof, the method comprising administering to the subject a therapeutically effective amount of the T cells obtainable by the method disclosed herein, thereby treating the pathology.

According to specific embodiments, the T cells are isolated from the immune cells prior to their adoptive transfer.

According to other specific embodiments, the T cells are not isolated from the immune cells prior to their adoptive transfer.

The cells used according to specific embodiments of the present invention may be autologous or non-autologous; they can be syngeneic or non-syngeneic: allogeneic or xenogeneic to the subject in need thereof; each possibility represents a separate embodiment of the present invention.

According to specific embodiments, the cells are autologous to the subject.

According to specific embodiments, the subject in need thereof suffers from the same pathology as the subject the immune cells were obtained from.

As used herein, the term “subject” includes mammals, e.g., human beings at any age and of any gender. According to specific embodiments, the term “subject” refers to a subject who suffers from the pathology (disease, disorder or medical condition).

According to specific embodiments, the subject is a human.

The term “treating” or “treatment” refers to inhibiting, preventing or arresting the development of a pathology (disease, disorder or medical condition) and/or causing the reduction, remission, or regression of a pathology or a symptom of a pathology. Those of skill in the art will understand that various methodologies and assays can be used to assess the development of a pathology, and similarly, various methodologies and assays may be used to assess the reduction, remission or regression of a pathology.

Non-limiting examples of diseases that can benefit from adoptive T cell therapy include hyper-proliferative diseases, diseases associated with immune suppression, autoimmune diseases and infection.

According to specific embodiments, the disease comprises a hyper-proliferative disease.

According to specific embodiments, the hyper-proliferative disease comprises sclerosis, fibrosis, Idiopathic pulmonary fibrosis, psoriasis, systemic sclerosis/scleroderma, primary biliary cholangitis, primary sclerosing cholangitis, liver fibrosis, prevention of radiation-induced pulmonary fibrosis, myelofibrosis or retroperitoneal fibrosis.

According to other specific embodiments, the hyper-proliferative disease comprises cancer.

As used herein, the term cancer encompasses both malignant and pre-malignant cancers.

With regard to pre-malignant or benign forms of cancer, optionally the compositions and methods thereof may be applied for halting the progression of the pre-malignant cancer to a malignant form.

Cancers which can be treated by the methods of some embodiments of the invention can be any solid or non-solid cancer and/or cancer metastasis.

According to specific embodiments, the cancer comprises malignant cancer.

Cancers which can be treated by the methods of some embodiments of the invention can be any solid or non-solid cancer and/or cancer metastasis. Examples of cancer include but are not limited to, carcinoma, lymphoma, blastoma, sarcoma, and leukemia. More particular examples of such cancers include squamous cell cancer, lung cancer (including small-cell lung cancer, non-small-cell lung cancer, adenocarcinoma of the lung, and squamous carcinoma of the lung), cancer of the peritoneum, hepatocellular cancer, gastric or stomach cancer (including gastrointestinal cancer), pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney or renal cancer, liver cancer, prostate cancer, vulval cancer, thyroid cancer, hepatic carcinoma and various types of head and neck cancer, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; Burkitt lymphoma, Diffused large B cell lymphoma (DLBCL), high grade lymphoblastic NHL; high-grade small non-cleaved cell NHL; bulky disease NHL; mantle cell lymphoma; AIDS-related lymphoma; and Waldenstrom's Macroglobulinemia); T cell lymphoma, Hodgkin lymphoma, chronic lymphocytic leukemia (CLL); acute lymphoblastic leukemia (ALL); Acute myeloid leukemia (AML), Acute promyelocytic leukemia (APL), Hairy cell leukemia; chronic myeloblastic leukemia (CML); and post-transplant lymphoproliferative disorder (PTLD), as well as abnormal vascular proliferation associated with phakomatoses, edema (such as that associated with brain tumors), and Meigs' syndrome. Preferably, the cancer is selected from the group consisting of breast cancer, colorectal cancer, rectal cancer, non-small cell lung cancer, non-Hodgkins lymphoma (NHL), renal cell cancer, prostate cancer, liver cancer, pancreatic cancer, soft-tissue sarcoma, Kaposi's sarcoma, carcinoid carcinoma, head and neck cancer, melanoma, ovarian cancer, mesothelioma, and multiple myeloma. The cancerous conditions amenable for treatment of the invention include metastatic cancers.

According to specific embodiments, the cancer comprises pre-malignant cancer.

Pre-malignant cancers (or pre-cancers) are well characterized and known in the art (refer, for example, to Berman J J. and Henson D E., 2003. Classifying the precancers: a metadata approach. BMC Med Inform Decis Mak. 3:8). Classes of pre-malignant cancers amenable to treatment via the method of the invention include acquired small or microscopic pre-malignant cancers, acquired large lesions with nuclear atypia, precursor lesions occurring with inherited hyperplastic syndromes that progress to cancer, and acquired diffuse hyperplasias and diffuse metaplasias. Examples of small or microscopic pre-malignant cancers include HGSIL (High grade squamous intraepithelial lesion of uterine cervix), AIN (anal intraepithelial neoplasia), dysplasia of vocal cord, aberrant crypts (of colon), PIN (prostatic intraepithelial neoplasia). Examples of acquired large lesions with nuclear atypia include tubular adenoma, AILD (angioimmunoblastic lymphadenopathy with dysproteinemia), atypical meningioma, gastric polyp, large plaque parapsoriasis, myelodysplasia, papillary transitional cell carcinoma in-situ, refractory anemia with excess blasts, and Schneiderian papilloma. Examples of precursor lesions occurring with inherited hyperplastic syndromes that progress to cancer include atypical mole syndrome, C cell adenomatosis and MEA. Examples of acquired diffuse hyperplasias and diffuse metaplasias include AIDS, atypical lymphoid hyperplasia, Paget's disease of bone, post-transplant lymphoproliferative disease and ulcerative colitis.

According to specific embodiments, the cancer is Acute Lymphocytic Leukemia (ALL), Acute Myeloid Leukemia, Anal Cancer, Basal Cell Carcinoma, B-Cell Non-Hodgkin Lymphoma, Bile Duct Cancer, Bladder Cancer, Breast Cancer, Cervical Cancer, Chronic Lymphocytic Leukemia (CLL), Chronic Myelocytic Leukemia (CML), Colorectal Cancer, Cutaneous T-Cell Lymphoma, Diffuse Large B-Cell Lymphoma, Endometrial Cancer, Esophageal Cancer, Fallopian Tube Cancer, Follicular Lymphoma, Gastric Cancer, Gastroesophageal (GE) Junction Carcinomas, Germ Cell Tumors, Germinomatous (Seminomatous), Germ Cell Tumors, Glioblastoma Multiforme (GBM), Gliosarcoma, Head And Neck Cancer, Hepatocellular Carcinoma, Hodgkin Lymphoma, Hypopharyngeal Cancer, Laryngeal Cancer, Leiomyosarcoma, Mantle Cell Lymphoma, Melanoma, Merkel Cell Carcinoma, Multiple Myeloma, Neuroendocrine Tumors, Non-Hodgkin Lymphoma, Non-Small Cell Lung Cancer, Oral Cavity (Mouth) Cancer, Oropharyngeal Cancer, Osteosarcoma, Ovarian Cancer, Pancreatic Cancer, Peripheral Nerve Sheath Tumor (Neurofibrosarcoma), Peripheral T-Cell Lymphomas (PTCL), Peritoneal Cancer, Prostate Cancer, Renal Cell Carcinoma, Salivary Gland Cancer, Skin Cancer, Small-Cell Lung Cancer, Soft Tissue Sarcoma, Squamous Cell Carcinoma, Synovial Sarcoma, Testicular Cancer, Thymic Carcinoma, Thyroid Cancer, Ureter Cancer, Urethral Cancer, Uterine Cancer, Vaginal Cancer or Vulvar Cancer.

According to specific embodiments, the cancer is Acute myeloid leukemia, Bladder Cancer, Breast Cancer, chronic lymphocytic leukemia, Chronic myelogenous leukemia, Colorectal cancer, Diffuse large B-cell lymphoma, Epithelial Ovarian Cancer, Epithelial Tumor, Fallopian Tube Cancer, Follicular Lymphoma, Glioblastoma multiform, Hepatocellular carcinoma, Head and Neck Cancer, Leukemia, Lymphoma, Mantle Cell Lymphoma, Melanoma, Mesothelioma, Multiple Myeloma, Nasopharyngeal Cancer, Non Hodgkin lymphoma, Non-small-cell lung carcinoma, Ovarian Cancer, Prostate Cancer or Renal cell carcinoma.

According to specific embodiments, the cancer is selected from the group consisting of Acute Lymphocytic Leukemia (ALL), Bladder Cancer, Breast Cancer, Colorectal Cancer, Head and Neck Cancer, Hepatocellular Carcinoma, Melanoma, Multiple Myeloma, Non-Small Cell Lung Cancer, Non-Hodgkin Lymphoma, Ovarian Cancer, Renal Cell Carcinoma.

According to specific embodiments, the cancer is selected from the group consisting of Gastrointestinal (GI) cancers, Breast Cancer, Ovarian Cancer and Pancreatic Cancer.

According to specific embodiments, the cancer is selected from the group consisting of pancreatic cancer, lung cancer, colon cancer and leukemia.

According to specific embodiments, the cancer is pancreatic cancer.

According to specific embodiments, the cancer is lung cancer.

According to specific embodiments, the lung cancer is non-small cell lung cancer (NSCLC).

According to specific embodiments, the NSCLC is squamous cells carcinoma.

According to specific embodiments, the NSCLC is adenocarcinoma.

According to specific embodiments, the cancer is colon cancer with metastasis e.g. to the lungs.

According to specific embodiments, the cancer is leukemia.

According to specific embodiments, the leukemia is acute myeloid leukemia (AML).

According to specific embodiments, the disease comprises a disease associated with immune suppression.

According to specific embodiments, the disease comprises HIV, Measles, influenza, LCCM, RSV, Human Rhinoviruses, EBV, CMV or Parvo viruses.

According to specific embodiments, the disease comprises an infection.

As used herein, the term “infection” or “infectious disease” refers to a disease induced by a pathogen. Specific examples of pathogens include, viral pathogens, bacterial pathogens e.g., intracellular mycobacterial pathogens (such as, for example, Mycobacterium tuberculosis), intracellular bacterial pathogens (such as, for example, Listeria monocytogenes), or intracellular protozoan pathogens (such as, for example, Leishmania and Trypanosoma).

Specific types of viral pathogens causing infectious diseases treatable according to specific embodiments of the present invention include, but are not limited to, retroviruses, circoviruses, parvoviruses, papovaviruses, adenoviruses, herpesviruses, iridoviruses, poxviruses, hepadnaviruses, picornaviruses, caliciviruses, togaviruses, flaviviruses, reoviruses, orthomyxoviruses, paramyxoviruses, rhabdoviruses, bunyaviruses, coronaviruses, arenaviruses, and filoviruses.

Specific examples of viral infections which may be treated according to specific embodiments of the present invention include, but are not limited to, human immunodeficiency virus (HIV)-induced acquired immunodeficiency syndrome (AIDS), influenza, rhinoviral infection, viral meningitis, Epstein-Barr virus (EBV) infection, hepatitis A, B or C virus infection, measles, papilloma virus infection/warts, cytomegalovirus (CMV) infection, Herpes simplex virus infection, yellow fever, Ebola virus infection, rabies, etc.

According to specific embodiments, the disease is an autoimmune disease. Such autoimmune diseases include, but are not limited to, cardiovascular diseases, rheumatoid diseases, glandular diseases, gastrointestinal diseases, cutaneous diseases, hepatic diseases, neurological diseases, muscular diseases, nephric diseases, diseases related to reproduction, connective tissue diseases and systemic diseases.

Examples of autoimmune cardiovascular diseases include, but are not limited to atherosclerosis (Matsuura E. et al., Lupus. 1998; 7 Suppl 2:S135), myocardial infarction (Vaarala O. Lupus. 1998; 7 Suppl 2:S132), thrombosis (Tincani A. et al., Lupus 1998; 7 Suppl 2:S107-9), Wegener's granulomatosis, Takayasu's arteritis, Kawasaki syndrome (Praprotnik S. et al., Wien Klin Wochenschr 2000 Aug. 25; 112 (15-16):660), anti-factor VIII autoimmune disease (Lacroix-Desmazes S. et al., Semin Thromb Hemost. 2000; 26 (2):157), necrotizing small vessel vasculitis, microscopic polyangiitis, Churg and Strauss syndrome, pauci-immune focal necrotizing and crescentic glomerulonephritis (Noel L H. Ann Med Interne (Paris). 2000 May; 151 (3):178), antiphospholipid syndrome (Flamholz R. et al., J Clin Apheresis 1999; 14 (4):171), antibody-induced heart failure (Wallukat G. et al., Am J Cardiol. 1999 Jun. 17; 83 (12A):75H), thrombocytopenic purpura (Moccia F. Ann Ital Med Int. 1999 April-June; 14 (2):114; Semple J W. et al., Blood 1996 May 15; 87 (10):4245), autoimmune hemolytic anemia (Efremov D G. et al., Leuk Lymphoma 1998 January; 28 (3-4):285; Sallah S. et al., Ann Hematol 1997 March; 74 (3):139), cardiac autoimmunity in Chagas' disease (Cunha-Neto E. et al., J Clin Invest 1996 Oct. 15; 98 (8):1709) and anti-helper T lymphocyte autoimmunity (Caporossi A P. et al., Viral Immunol 1998; 11 (1):9).

Examples of autoimmune rheumatoid diseases include, but are not limited to rheumatoid arthritis (Krenn V. et al., Histol Histopathol 2000 July; 15 (3):791; Tisch R, McDevitt H O. Proc Natl Acad Sci units S A 1994 Jan. 18; 91 (2):437) and ankylosing spondylitis (Jan Voswinkel et al., Arthritis Res 2001; 3 (3): 189).

Examples of autoimmune glandular diseases include, but are not limited to, pancreatic disease, Type I diabetes, thyroid disease, Graves' disease, thyroiditis, spontaneous autoimmune thyroiditis, Hashimoto's thyroiditis, idiopathic myxedema, ovarian autoimmunity, autoimmune anti-sperm infertility, autoimmune prostatitis and Type I autoimmune polyglandular syndrome. Such diseases include, but are not limited to autoimmune diseases of the pancreas, Type 1 diabetes (Castano L. and Eisenbarth G S. Ann. Rev. Immunol. 8:647; Zimmet P. Diabetes Res Clin Pract 1996 October; 34 Suppl:S125), autoimmune thyroid diseases, Graves' disease (Orgiazzi J. Endocrinol Metab Clin North Am 2000 June; 29 (2):339; Sakata S. et al., Mol Cell Endocrinol 1993 March; 92 (1):77), spontaneous autoimmune thyroiditis (Braley-Mullen H. and Yu S, J Immunol 2000 Dec. 15; 165 (12):7262), Hashimoto's thyroiditis (Toyoda N. et al., Nippon Rinsho 1999 August; 57 (8):1810), idiopathic myxedema (Mitsuma T. Nippon Rinsho. 1999 August; 57 (8):1759), ovarian autoimmunity (Garza K M. et al., J Reprod Immunol 1998 February; 37 (2):87), autoimmune anti-sperm infertility (Diekman A B. et al., Am J Reprod Immunol. 2000 March; 43 (3):134), autoimmune prostatitis (Alexander R B. et al., Urology 1997 December; 50 (6):893) and Type I autoimmune polyglandular syndrome (Hara T. et al., Blood. 1991 Mar. 1; 77 (5):1127).

Examples of autoimmune gastrointestinal diseases include, but are not limited to, chronic inflammatory intestinal diseases (Garcia Herola A. et al., Gastroenterol Hepatol. 2000 January; 23 (1):16), celiac disease (Landau Y E. and Shoenfeld Y. Harefuah 2000 Jan. 16; 138 (2):122), colitis, ileitis and Crohn's disease.

Examples of autoimmune cutaneous diseases include, but are not limited to, autoimmune bullous skin diseases, such as, but are not limited to, pemphigus vulgaris, bullous pemphigoid and pemphigus foliaceus.

Examples of autoimmune hepatic diseases include, but are not limited to, hepatitis, autoimmune chronic active hepatitis (Franco A. et al., Clin Immunol Immunopathol 1990 March; 54 (3):382), primary biliary cirrhosis (Jones D E. Clin Sci (Colch) 1996 November; 91 (5):551; Strassburg C P. et al., Eur J Gastroenterol Hepatol. 1999 June; 11 (6):595) and autoimmune hepatitis (Manns M P. J Hepatol 2000 August; 33 (2):326).

Examples of autoimmune neurological diseases include, but are not limited to, multiple sclerosis (Cross A H. et al., J Neuroimmunol 2001 Jan. 1; 112 (1-2):1), Alzheimer's disease (Oron L. et al., J Neural Transm Suppl. 1997; 49:77), myasthenia gravis (Infante A J. And Kraig E, Int Rev Immunol 1999; 18 (1-2):83; Oshima M. et al., Eur J Immunol 1990 December; 20 (12):2563), neuropathies, motor neuropathies (Kornberg A J. J Clin Neurosci. 2000 May; 7 (3):191); Guillain-Barre syndrome and autoimmune neuropathies (Kusunoki S. Am J Med Sci. 2000 April; 319 (4):234), myasthenia, Lambert-Eaton myasthenic syndrome (Takamori M. Am J Med Sci. 2000 April; 319 (4):204); paraneoplastic neurological diseases, cerebellar atrophy, paraneoplastic cerebellar atrophy and stiff-man syndrome (Hiemstra H S. et al., Proc Natl Acad Sci units S A 2001 Mar. 27; 98 (7):3988); non-paraneoplastic stiff man syndrome, progressive cerebellar atrophies, encephalitis, Rasmussen's encephalitis, amyotrophic lateral sclerosis, Sydeham chorea, Gilles de la Tourette syndrome and autoimmune polyendocrinopathies (Antoine J C. and Honnorat J. Rev Neurol (Paris) 2000 January; 156 (1):23); dysimmune neuropathies (Nobile-Orazio E. et al., Electroencephalogr Clin Neurophysiol Suppl 1999; 50:419); acquired neuromyotonia, arthrogryposis multiplex congenita (Vincent A. et al., Ann N Y Acad Sci. 1998 May 13; 841:482), neuritis, optic neuritis (Soderstrom M. et al., J Neurol Neurosurg Psychiatry 1994 May; 57 (5):544) and neurodegenerative diseases.

Examples of autoimmune muscular diseases include, but are not limited to, myositis, autoimmune myositis and primary Sjogren's syndrome (Feist E. et al., Int Arch Allergy Immunol 2000 September; 123 (1):92) and smooth muscle autoimmune disease (Zauli D. et al., Biomed Pharmacother 1999 June; 53 (5-6):234).

Examples of autoimmune nephric diseases include, but are not limited to, nephritis and autoimmune interstitial nephritis (Kelly C J. J Am Soc Nephrol 1990 August; 1 (2):140).

Examples of autoimmune diseases related to reproduction include, but are not limited to, repeated fetal loss (Tincani A. et al., Lupus 1998; 7 Suppl 2:S107-9).

Examples of autoimmune connective tissue diseases include, but are not limited to, ear diseases, autoimmune ear diseases (Yoo T J. et al., Cell Immunol 1994 August; 157 (1):249) and autoimmune diseases of the inner ear (Gloddek B. et al., Ann N Y Acad Sci 1997 Dec. 29; 830:266).

Examples of autoimmune systemic diseases include, but are not limited to, systemic lupus erythematosus (Erikson J. et al., Immunol Res 1998; 17 (1-2):49) and systemic sclerosis (Renaudineau Y. et al., Clin Diagn Lab Immunol. 1999 March; 6 (2):156); Chan O T. et al., Immunol Rev 1999 June; 169:107).

According to specific embodiments, the disease is graft rejection disease. Examples of diseases associated with transplantation of a graft include, but are not limited to, graft rejection, chronic graft rejection, subacute graft rejection, hyperacute graft rejection, acute graft rejection and graft versus host disease.

According to specific embodiments, the disease is an allergic disease. Examples of allergic diseases include, but are not limited to, asthma, hives, urticaria, pollen allergy, dust mite allergy, venom allergy, cosmetics allergy, latex allergy, chemical allergy, drug allergy, insect bite allergy, animal dander allergy, stinging plant allergy, poison ivy allergy and food allergy.

According to specific embodiments, the immune cells or T cells disclosed herein can be administered to a subject in combination with other established or experimental therapeutic regimen to treat the disease (e.g., before, simultaneously or following) including, but not limited to analgesics, chemotherapeutic agents, radiotherapeutic agents, cytotoxic therapies (e.g. immunoablative agents, conditioning), hormonal therapy, antibodies, immunosuppressive agents and other treatment regimens (e.g., surgery) which are well known in the art.

According to specific embodiments, the immune cells or T cells disclosed herein can be administered to a subject in combination with adoptive cell transplantation such as, but not limited to transplantation of bone marrow cells, hematopoietic stem cells, PBMCs, cord blood stem cells and/or induced pluripotent stem cells.

The immune cells or T cells of some embodiments of the invention can be administered to an organism per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the immune cells or T cells of some embodiments of the invention accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences”, Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method. Alternately, one may administer the pharmaceutical composition in a local rather than systemic manner, for example, via injection of the pharmaceutical composition directly into a tissue region of a patient.

According to one embodiment, the route of administration includes, for example, an injection, ingestion, transfusion, implantation or transplantation. The compositions described herein may be administered to a patient subcutaneously, intradermally, intratumorally, intranodally, intramedullary, intramuscularly, by intravenous (i.v.) injection, or intraperitoneally. In one embodiment, the pharmaceutical composition of the present invention is administered to a patient by intradermal or subcutaneous injection. In another embodiment, the pharmaceutical composition of the present invention is preferably administered by i.v. injection. The pharmaceutical composition may be injected directly into a tumor, lymph node, or site of infection.

Pharmaceutical compositions of some embodiments of the invention may be manufactured by processes well known in the art, e.g., by means of conventional mixing, dissolving, granulating, dragee-making, levitating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer.

For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion.

Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes.

Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of the invention include compositions wherein the active ingredients are contained in an amount effective to achieve the intended purpose. More specifically, a therapeutically effective amount means an amount of active ingredients effective to prevent, alleviate or ameliorate symptoms of a pathology or prolong the survival of the subject being treated.

Determination of a therapeutically effective amount is well within the capability of those skilled in the art, especially in light of the detailed disclosure provided herein.

When “therapeutic amount” is indicated, the precise amount of the compositions of the present invention to be administered can be determined by a physician with consideration of individual differences in age, weight, disease state, e.g. tumor size, extent of infection or metastasis, and the condition of the patient (subject). According to specific embodiments, a pharmaceutical composition comprising the T cells described herein may be administered at a dosage of 1×10⁵ to 1×10¹⁰ cells/kg body weight, including all integer values within this range.

The cell compositions of some embodiments of the invention may also be administered multiple times at these dosages. Sometimes, to investigate the patient's tolerance, the total number of cells is injected over three courses, e.g. 10% on the first day, 30% on the second day, and 60% on the third day (see e.g. Lijun Zhao and Yu J. Cao, Front Immunol. 2019; 10: 2250).

The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human.

The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p.1).

Dosage amount and interval may be adjusted individually to provide levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

According to another aspect of the present invention there is provided an article of manufacture comprising a packaging material packaging a non-cellular agent capable of binding 4-1BB and activating said 4-1BB signaling pathway and at least one cytokine selected from the group consisting of IL-2, IL-15 and IL-21.

According to an additional or an alternative aspect of the present invention there is provided an article of manufacture comprising a packaging material packaging a non-cellular agent capable of binding:

(i) 4-1BB and activating said 4-1BB signaling pathway; and

(ii) a ligand or a receptor of a type I membrane protein,

and at least one cytokine selected from the group consisting of IL-2, IL-15 and IL-21.

According to an additional or an alternative aspect of the present invention there is provided an article of manufacture comprising a packaging material packaging a non-cellular agent capable of binding 4-1BB and activating said 4-1BB signaling pathway, a non-cellular agent capable of binding a ligand or a receptor of a type I membrane protein and at least one cytokine selected from the group consisting of IL-2, IL-15 and IL-21.

According to specific embodiments, the article of manufacture comprises IL-2, IL-15 and IL-21.

According to specific embodiments, the non-cellular agent and the at least one cytokine are packaged in separate containers.

According to specific embodiments, the non-cellular agent and the at least one cytokine are packaged in a co-formulation.

According to another aspect of the present invention there is provided an article of manufacture comprising a packaging material packaging a non-cellular agent capable of binding 4-1BB and activating said 4-1BB signaling pathway and a nucleic acid sequence encoding a chimeric antigen receptor (CAR).

According to another aspect of the present invention there is provided an article of manufacture comprising a packaging material packaging a non-cellular agent capable of binding:

(i) 4-1BB and activating said 4-1BB signaling pathway; and

(ii) a ligand or a receptor of a type I membrane protein,

and a nucleic acid sequence encoding a chimeric antigen receptor (CAR).

According to another aspect of the present invention there is provided an article of manufacture comprising a packaging material packaging a non-cellular agent capable of binding 4-1BB and activating said 4-1BB signaling pathway, a non-cellular agent capable of binding a ligand or a receptor of a type I membrane protein, and a nucleic acid sequence encoding a chimeric antigen receptor (CAR).

According to specific embodiments, the non-cellular agent and the nucleic acid sequence encoding a CAR are packaged in separate containers.

According to specific embodiments, the non-cellular agent and the nucleic acid sequence encoding a CAR are packaged in a co-formulation.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Culturing PBMCs in the Presence of PD1-4-1BBL Enriches for Tumor Reactive T Cell Clones

Materials

PD1-4-1BBL fusion protein (SEQ ID NO: 49) having a His tag at its N′ terminal, Ficoll-Paque (cat #17-1440-03, GE Healthcare), Serum-free Dendritic Cell Medium (cat #20801-0100, CellGernix), Pooled human AB serum (cat #IPLA-SerAB-13458, Inonative Research), hIL-2 (cat #cyt-209-b, Prospec), hIL-15 (cat #cyt-230b, Prospec), hIL-21 (cat #cyt-408-b, Prospec), Penicillin-Strepomycin (cat #15140-122, Gibco), Amphotericin B solution (cat #A2942, Sigma), Bioworld Ciprofloxacin (Cipro) Hydrochloride (cat #50255729, Fisher Scientific), RPMI 1640 (cat #61870-044, Glico), Fetal bovine serum (cat #26140-079, Glico), human IFN-7 pre coated ELISA kit (cat #Bgk01579, Biogems), anti-CD4 antibody (cat #317436,Biolegend), anti-CD8 antibody (cat #347313, BD), DNA extraction kit (Cat #51104, QIAGEN)

Methods

Human PBMCs were isolated from peripheral blood of four pancreatic cancer patients using Ficoll. Following isolation, the PBMCs were cultured in serum-free dendritic cell medium supplemented with 1% Penicillin-streptomycin, 1% Amphotericin B, 0.1% Ciprofloxacin, 10% human AB serum, hIL-2 (1000 u/mL), hIL-15 (10 ng/mL) and hIL-21(10 ng/mL) for 10 days to enrich the T cell population. In the next step, the cells obtained from two of the patients (namely patients 3 and 10) were seeded in the same media with the addition of PD1-4-1BBL fusion protein (1 μg/mL) or an autologous organoid for additional 14 days, with refreshment of media and PD1-4-1BBL on day 7. Generation of the autologous organoids was effects as described in Huang et al., Nat Med. 2015 21(11):1364-71. PBMCs cultured under the same conditions without PD1-4-1BBL or autologous organoid were used as a negative control. Media was collected on days 7 and 14 for quantification of IFN-7 using a human IFN-7 pre coated ELISA kit (cat #Bgk01579, Biogems). Following culturing, cells were collected, DNA was extracted using DNA extraction kit (Cat #51104, QIAGEN).

TCR deep sequencing (ImmunoSEQ) was performed by Adaptive Biotechnologies on genomic DNA. TCR sequencing was effected on 100,000-200000 reads per sample and analysis was effected using ImmunoSEQ, Analyses 3.0 (Adaptive Biotechnologies).

Results

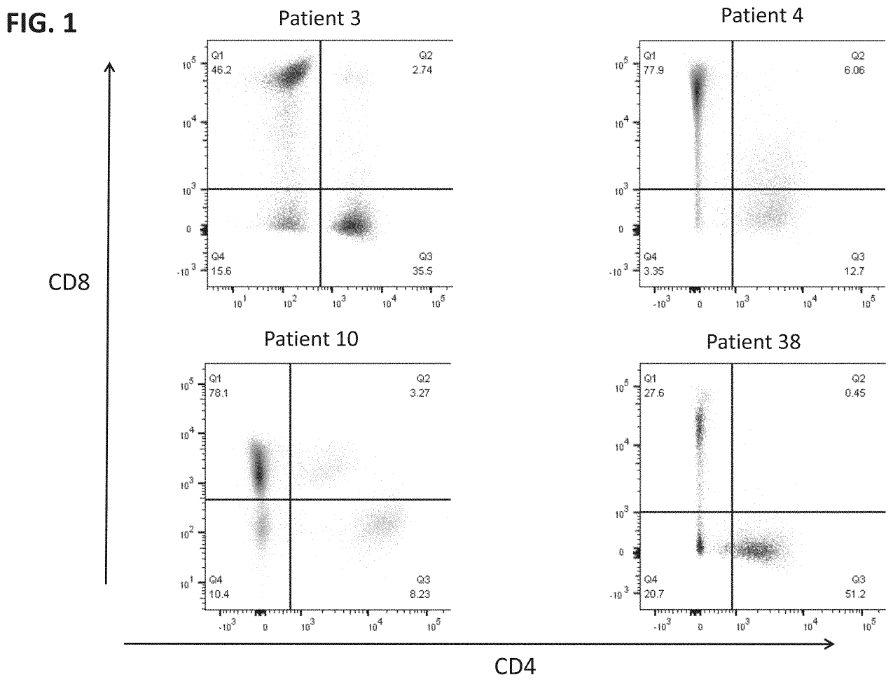

Following an initial 10 days period of culturing isolated PBMCs obtained from pancreatic cancer patients in a media containing hIL-2, hIL-15 and hIL-21, the cell population was highly enriched for T-cells, with more than 75% CD8 cytotoxic and CD4 helper T-cells (FIG. 1).

Addition of PD1-4-1BBL fusion protein to a second 14 days culturing period resulted in enrichment of specific T-cell clones as can be observed by the TCR sequencing (Table 1A hereinbelow). In patient #3, the two most abundant clones represent 90% of the total population and in patient #10, the 4 most abundant clones represent 70% of the population; while, in comparison, in both patients the 10 most abundant clones in the negative control culture (not comprising PD1-4-1BBL fusion protein nor organoid, Table 1C hereinbelow) represent ˜25% and ˜9%, respectively. Similarly, co-culturing the cells in the second 14 days culturing period with an autologous organoid resulted in enrichment of specific T-cell clones as well (Table 1B hereinbelow); with the 4 most abundant clones in patient #3 representing ˜63% of the cells and the 2 most abundant clones in patient #10 representing 84% of the population.

Moreover, there was an overlap between the highest represented clones in the cells cultured in the presence of the PD1-4-1BBL fusion protein and the cells co-cultured with the autologous organoids, indicating that binding of the PD1-4-1BBL protein to 4-1BB expressed on activated T-cells, enabled enrichment of tumor reactive T-cell clones. Specifically, in patient #3 the most abundant clone was identical and in patient #10 the second abundant clone was identical. Such clones were not identified as one of the 10 most abundant clones in the negative control culture.

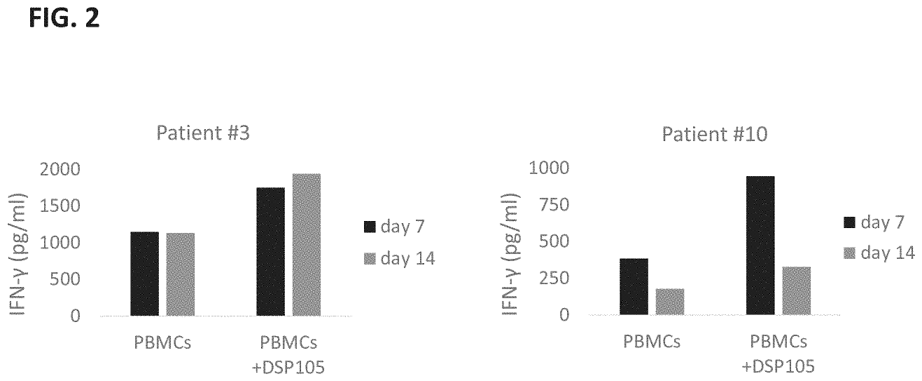

The level of IFNγ in the cell media was similar when the PBMCs were cultured in the presence of PD1-4-1BBL or co-cultured with the organoid and higher compared to the control cultured PBMCs. FIG. 2

TABLE 1A
The most 10 abundant TCR sequences and percentages following culturing of
PBMCs obtained from pancreatic cancer patients with PD1-4-1BBL fusion protein
(SEQ ID NO: 49)

| | Frequency | Number of | | SEQ | |
|---|---|---|---|---|---|
| Sample | (%) | templates | CDR3 sequence | ID NO: | TCR V beta |
| Pt3 | 72.0 | 41267 | CSARDPPSGRNGYTF | 102 | TCRBV20 |
| | 18.0 | 10308 | CASSPTIEETQYF | 103 | TCRBV06-01*01 |
| | 1.8 | 1037 | CASRSSNQPQHF | 104 | TCRBV19-01*01 |
| | 1.0 | 590 | CASSPRDSWTGELFF | 105 | TCRBV07-08*03 |
| | 0.8 | 469 | CASSANRAPNEKLFF | 106 | TCRBV27-01*01 |
| | 0.4 | 239 | CASRTTSGEQETQYF | 107 | TCRBV28-01*01 |
| | 0.4 | 211 | CASSYQVGDLGELFF | 108 | TCRBV07-06*01 |
| | 0.3 | 164 | CASSPPSGAVRTDTQYF | 109 | TCRBV07-06*01 |
| | 0.3 | 156 | CASSLATSSYNEQFF | 110 | TCRBV07-06*01 |
| | 0.2 | 131 | CASSRLGGLTQYF | 111 | TCRBV07-09*01 |
| Pt10 | 46.0 | 9770 | CASSQSATGNYEQYF | 112 | TCRBV04-01*01 |
| | 17.4 | 3684 | CASSYSIQGGMLSLGEKLFF | 113 | TCRBV06-02/06- |
| | | | | | 03*01 |
| | 3.8 | 809 | CATGLSVDTQYF | 114 | TCRBV06-05*01 |
| | 3.7 | 785 | CASSTAGAGYEQYF | 115 | TCRBV19-01*01 |
| | 1.2 | 245 | CASSITGGSGNEQFF | 116 | TCRBV19-01*01 |
| | 1.1 | 232 | CASSDRGMNTEAFF | 117 | TCRBV06-01*01 |
| | 0.9 | 182 | CASSFSAAQETQYF | 118 | TCRBV27-01*01 |
| | 0.7 | 159 | CASSRDREKLFF | 119 | TCRBV02-01 |
| | 0.7 | 152 | CASSKNNEQFF | 120 | TCRBV12-03/12- |
| | | | | | 04*01 |
| | 0.7 | 149 | CASSEGAGAKQPQHF | 121 | TCRBV02-01 |

TABLE 1B
The most 10 abundant TCR sequence following culturing of PBMCs obtained from
pancreatic cancer patients with an autologous organoid

| | Frequency | Number of | | SEQ ID | |
|---|---|---|---|---|---|
| Sample | (%) | templates | CDR3 sequence | NO: | TCR V beta |
| Pt3 | 33.1 | 9410 | CSARDPPSGRNGYTF | 102 | TCRBV20 |
| | 16.8 | 4782 | CASSLGGLAITDTQYF | 122 | TCRBV07-09*01 |
| | 7.5 | 2136 | CASSPTIEETQYF | 103 | TCRBV06-01*01 |
| | 5.8 | 1653 | CASSQMQGLVGEQYF | 123 | TCRBV04-02*01 |
| | 3.8 | 1081 | CASGDRGPRYEQYF | 124 | TCRBV21-01*01 |
| | 2.2 | 632 | CSAGTGTSGGRETQYF | 125 | TCRBV20 |
| | 2.2 | 627 | CASRTTSGEQETQYF | 107 | TCRBV28-01*01 |
| | 2.0 | 580 | CASSYQVGDLGELFF | 108 | TCRBV07-06*01 |
| | 2.0 | 557 | CASSSYTAKGRYNEQFF | 126 | TCRBV11-03*01 |
| | 1.7 | 473 | CSASPDSNQPQHF | 127 | TCRBV20 |
| Pt10 | 77.8 | 52272 | CASSKNNEQFF | 120 | TCRBV12-03/12- |
| | | | | | 04*01 |
| | 6.3 | 4253 | CASSYSIQGGMLSLGEKLFF | 113 | TCRBV06-02/06- |
| | | | | | 03*01 |
| | 2.6 | 1715 | CASSQSATGNYEQYF | 112 | TCRBV04-01*01 |
| | 1.8 | 1233 | CASSITGGSGNEQFF | 116 | TCRBV19-01*01 |
| | 1.2 | 794 | CASKVFSYTGELFF | 128 | TCRBV27-01*01 |
| | 1.0 | 690 | CASSFEGYNEQFF | 129 | TCRBV07-08*01 |
| | 0.9 | 620 | CASSLGDANNEQFF | 130 | TCRBV11 |
| | 0.7 | 495 | CASSSTIGGVNEQFF | 131 | TCRBV07-06*01 |
| | 0.7 | 440 | CASSLTGRAYMDYGYTF | 132 | TCRBV28-01*01 |
| | 0.6 | 423 | CASSRALRGHNGYTF | 133 | TCRBV03-01/03- |
| | | | | | 02*01 |

TABLE 1C
The most 10 abundant TCR sequence following culturing of PBMCs obtained from
pancreatic cancer patients with control media not containing PD1-4-1BBL fusion protein or
autologous organoid

| | Frequency | Number of | | SEQ ID | |
|---|---|---|---|---|---|
| Sample | (%) | templates | CDR3 sequence | NO: | TCR V beta |
| Pt3 | 8.6 | 13556 | CASSPTIEETQYF | 103 | TCRBV06-01*01 |
| | 2.7 | 4248 | CASSQVASTDTQYF | 134 | TCRBV03-01/03- |
| | | | | | 02*01 |
| | 2.5 | 3876 | CSARDPPSGRNGYTF | 102 | TCRBV20 |
| | 2.4 | 3816 | CASRSSNQPQHF | 104 | TCRBV19-01*01 |
| | 2.3 | 3549 | CASSSRDRMNTEAFF | 135 | TCRBV06-05*01 |
| | 1.6 | 2524 | CASSCMGQGEGQPQHF | 136 | TCRBV11-01*01 |
| | 1.6 | 2462 | CASSQQENQPQHF | 137 | TCRBV04-01*01 |
| | 1.3 | 1991 | CASSANSGNQETQYF | 138 | TCRBV07-07*01 |
| | 1.0 | 1519 | CASSLQSRSGEQYF | 139 | TCRBV05-04*01 |
| | 0.9 | 1412 | CASSLTSATGELFF | 140 | TCRBV28-01*01 |
| Pt10 | 2.6 | 5137 | CASSTAGAGYEQYF | 115 | TCRBV19-01*01 |
| | 1.0 | 1917 | CASSLGGLAANYEQYF | 141 | TCRBV05-01*01 |
| | 0.8 | 1599 | CASSLAVQGGGYTF | 142 | TCRBV05-01*01 |
| | 0.8 | 1501 | CASSGGGLAGNSEQYF | 143 | TCRBV05-01*01 |
| | 0.8 | 1473 | CASTLGQGAWPLHF | 144 | TCRBV06-05*01 |
| | 0.7 | 1415 | CASSVRPTDTDTQYF | 145 | TCRBV13-01*01 |
| | 0.6 | 1242 | CASSVGGNTEAFF | 146 | TCRBV09-01*01 |
| | 0.5 | 1020 | CASSGDYDEQFF | 147 | TCRBV07 |
| | 0.5 | 891 | CASSIRSGRVDEQFF | 148 | TCRBV19-01*01 |
| | 0.4 | 805 | CASSKTDSNQPQHF | 149 | TCRBV09-01*01 |

### Example 2

### Generation of Recombinant TCRs Having Sequences of the Enriched T Cells Clones

The TCR sequences of the top 2 T-cell clones generated following culturing with the PD1-4-1BBL fusion polypeptide as described in Example 1 hereinabove are used to generate two recombinant TCRs for each patient. As a control, two recombinant TCRs that are negatively selected during the process are generated. The recombinant TCRs are expressed in SKW (PMID: 340692) cells and populations of recombinant TCR expressing SKW cells are generated.

Following, the TCR expressing SKW cells are used to determine their response to Patient #3 or Patient #10 organoids. The recognition of the tumor cells is performed in assays such as ELISPOT and ELISA for IFN-7 release (Tan et al., J Immunother Cancer. 2019 Aug. 28; 7(1):232). Cytotoxic assays, to determine the killing effect of the clones on the matching tumor cells is performed by e.g. labelling the tumor cells with CFSE, co-culturing with the TCR expressing cells and measuring viability by flow cytometry assays (Tan et al., J Immunother Cancer. 2019 Aug. 28; 7(1):232).

### Example 3

### Culturing PBMCs in the Presence of PD1-4-1BBL Enriches for T Cells Having a Memory Phenotype

Materials

PD1-4-1BBL fusion protein (SEQ ID NO: 49) having a His tag at its N′ terminal, Ficoll-Paque (cat #17-1440-03, GE Healthcare), Serum-free Dendritic Cell Medium (cat #20801-0100, CellGernix), Pooled human AB serum (cat #IPLA-SerAB-13458, Inonative Research), hIL-2 (cat #cyt-209-b, Prospec), hIL-15 (cat #cyt-230b, Prospec), hIL-21 (cat #cyt-408-b, Prospec), Penicillin-Strepomycin (cat #15140-122, Gibco), Amphotericin B solution (cat #A2942, Sigma), Bioworld Ciprofloxacin (Cipro) Hydrochloride (cat #50255729, Fisher Scientific), RPMI 1640 (cat #61870-044, Glico), Fetal bovine serum (cat #26140-079, Glico), Cell-ID™ Cisplatin (Fluidigm,201064), Human TruStain FcX™ (Fc Receptor Blocking Solution) (Biolegend,422302), Maxpar Cell Staining Buffer (CSB) (Fluidigm #201068), Antibody staining panel (listed in Table 2), DNA Intercalator (Fluidigm, #201192A), Fix and Perm Buffer (Fluidigm, #201067), Q Four Element Calibration Beads (Fluidigm, #201078).

Methods

Human PBMCs were isolated from peripheral blood of pancreatic cancer patients using Ficoll. Following isolation, the PBMCs were cultured in serum-free dendritic cell medium supplemented with 1% Penicillin-streptomycin, 1% Amphotericin B, 0.1% Ciprofloxacin, 10% human AB serum, hIL-2 (1000 u/mL), hIL-15 (10 ng/mL) and hIL-21 (10 ng/mL) for 10 days to enrich the T cell population. In the next step, the cells were seeded in the same media with the addition of PD1-4-1BBL fusion protein (1 μg/mL) or an autologous organoid for additional 14 days, with refreshment of media and PD1-4-1BBL on day 7. Generation of the autologous organoids was effects as described in Huang et al., Nat Med. 2015 21(11):1364-71. PBMCs cultured under the same conditions without PD1-4-1BBL or autologous organoid were used as a negative control. Following culturing, cells were collected, and 1×10⁶ cells were used for CyTOF analysis with 28 different antibody markers. First, the cells were stained with Cell-ID™ Cisplatin to test the viability. Following blocking the Fc receptor (FcR), the cells were transferred into Maxpar Cell Staining Buffer (CSB) for antibody staining. Stained cells were washed twice with 2 mL CSB, and incubated overnight at 4° C. with 1 mL DNA Intercalator, which was diluted in a Fix and Perm Buffer at a final concentration of 125 nM, to facilitate the discrimination between singlets, doublets, and triplets. Prior to data acquisition, cell pellets were resuspended in CSB containing 10% EQ Four Element Calibration Beads and filtered through a 35-am membrane. The cells were analysed by Helios with WB injector (Fluidigm) through several runs at an acquisition rate of ˜500 events per second. Following the manufacturer's instructions, settings were on default. The short-term signal fluctuations of Helios were normalized with EQ bead signals prior to data export and analysis. Abundance values obtained by mass cytometry were transformed using a scaled arcsine with a factor of 5, which diminished near-zero noise values in the measurements. Surface marker expression in each channel was normalized based on the signal intensity at the 99.5th percentile across all samples, thus yielding expression values as x-fold of the 99.5th percentile values. A series of gates for memory T cell subpopulations were based on the expression of: (1) CD103+, Tissue-resident memory T cell (CD103+, TRM): CD3+, CD45RO+, CD69+ and CD103+; (2) Tissue-resident memory T cell (TRM): CD3+, CD45RO+, CD69+ and CD103− and (3) Effect memory T cell (TEM): CD3+, CD45RO+, CD69− and CCR7−.

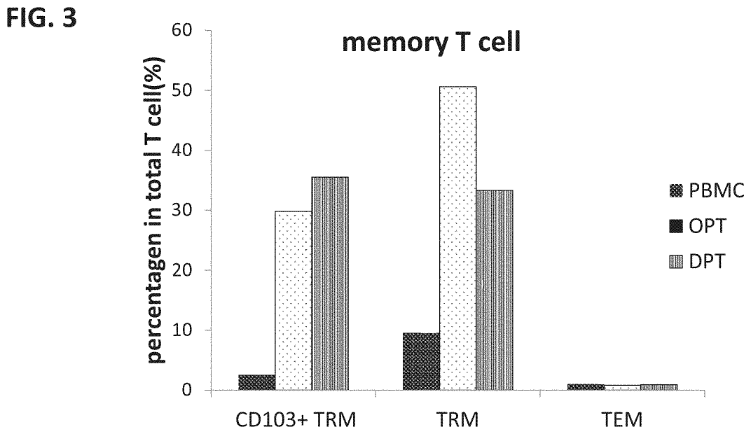

Results Culturing the PBMCs in the presence of the PD1-4-1BBL fusion protein resulted in enrichment of T cells with memory phenotype similar to culturing the PBMCs with the organoids (FIG. 3 and Table 2 hereinbelow). The CD3+ TRM population in the PD1-4-1BBL treated cells was >35% while with the organoids it was 30%. TRM subpopulation reached >30% in the PD1-4-1BBL treated cells with ˜50% in the organoid culture, indicating high enrichment of T-cells with memory phenotype.

TABLE 2
Abundance of memory T cells

| | Cell number | | | Percentage of total T cells (%) | | |
|---|---|---|---|---|---|---|
| | | | PBMCs + | | | PBMCs + |
| | | PBMCs + | PD1- | | PBMCs + | PD1- |
| Markers | PBMCs | organoids | 4-1BBL | PBMCs | organoids | 4-1BBL |
| CD3+ | 38600 | 42861 | 36599 | | | |
| CD3+/CD45RO+/CD69+/CD103+ | 962 | 12756 | 12985 | 2.5 | 29.8 | 35.5 |
| CD3+/CD45RO−/CD69+/CD103− | 3667 | 21708 | 12194 | 9.5 | 50.6 | 33.3 |
| CD3+/CD45RO+/CD69−/CCR7− | 384 | 335 | 314 | 1 | 0.8 | 0.9 |

### Example 4

### The In-Vivo Anti-Tumor Effect of the Enriched T Cells Clones

Autologous resected cancer samples are xenografted and expanded in nude mice to develop cohorts of tumor-bearing mice (Rubio-Viqueira et al., Clin Cancer Res. 2006 Aug. 1; 12(15):4652-61). The xenografted tumors maintain their fundamental genotypic features despite serial passages and recapitulate the genetic heterogeneity of the original cancer tissue. These tumor-bearing mice are used to evaluate the anti-tumor activity of the T-cells generated following culturing with the PD1-4-1BBL fusion protein compared to their controls, by injecting them to the mice, and evaluating tumor growth inhibition and mice survival.

### Example 5

### Culturing PBMCs in the Presence of PD1-4-1BBL, SIRPα-4-1BBL or Agonist 4-1BB Antibody Enriches T Cell Sub-Populations

Materials

PD1-4-1BBL fusion protein (SEQ ID NO: 49) having a His tag at its N′ terminal, PD1-4-1BBL fusion protein (SEQ ID NO: 74), SIRPα-4-1BBL fusion protein (SEQ ID NO: 89), agonist anti-4-1BB antibody, “Urelumab-like” (GenScript, U5656DG120S01/P70011808), Ficoll-Paque (cat #17-1440-03, GE Healthcare), Serum-free Dendritic Cell Medium (cat #20801-0100, CellGernix), Pooled human AB serum (cat #IPLA-SerAB-13458, Inonative Research), hIL-2 (cat #cyt-209-b, Prospec), hIL-15 (cat #cyt-230b, Prospec), hIL-21 (cat #cyt-408-b, Prospec), Penicillin-Strepomycin (cat #15140-122, Gibco), Amphotericin B solution (cat #A2942, Sigma), RPMI 1640 (cat #61870-044, Glico), Fetal bovine serum (cat #26140-079, Glico), 0.5% Trypan Blue Solution (catt #03-102-1B, Biological Industries), Fixable VioBlue viability stain 450 (cat #562247, BD), human trustain FcX (cat #422302, BioLegend), Briliant Stain Buffer (cat #566385, BD), and a kit for TCR Vβ families: Beta Mark TCR Vbeta Repertoire Kit (cat #IM3497, Beckman Coulter) and APC/Cy7 anti human CD8 (cat #301016, BioLegend).

Methods

Cell growth—Human PBMCs were isolated from peripheral blood of six non-small cell lung cancer (NSCLC) patients (OL #1-5, OL #9; OL #1 squamous cell carcinoma and the rest are adenocarcinoma) and two colon cancer patients with metastasis to the lung (OL #6 and OL #8), using Ficoll gradient according to the manufacturer's instructions. In parallel, a bone marrow sample from AML patient was also studied (AML #6). Following isolation, the PBMCs or the bone marrow sample were cultured in serum-free dendritic cell medium supplemented with 1% Penicillin-streptomycin, 1% Amphotericin B, 10% human AB serum, hIL-2 (1000 u/mL), hIL-(10 ng/mL) and hIL-21 (10 ng/mL). Cells were seeded in a 24-wells plate at a concentration of 1×10⁶ cells per well and cultured for three days (for group 1) or 5-7 days (for group 2) to enrich the T cell population (expansion phase). In the next step, the PD1-4-1BBL fusion protein (SEQ ID NO: 49 or 74, 1 μg/mL), SIRPα-4-1BBL fusion protein (SEQ ID NO: 89, 1 μg/mL), or the 4-1BB antibody (0.5 or 2 μg/mL), was added for additional 8-9 days (for group 2) or 11-13 days (for group 1), with refreshment of media, including cytokines and proteins/antibody every 2-3 days (treatment phase). Cell growth was monitored every 2-3 days by live cell counting, using trypan blue. When the cells reached 80-90% confluency, they were passaged by splitting 1:2 or 1:3 into a new 24-wells plate.

TCR Vβ repertoire analysis—Following 3 and 14-16 days in culture, cells were collected and analysed for TCR Vβ repertoire by flow cytometry, using Beckman Coulter kit. The kit allows studying the repertoire on T-cell subsets, using additional T-cell marker, CD8, conjugated to a different fluorophore. The kit is composed of 8 vials containing mixtures of conjugated TCR Vβ antibodies corresponding to 24 different specificities (about 70% coverage of normal human TCR Vβ repertoire). The frequency of each specific TCR Vβ was calculated separately for the CD8⁺ T-cells or the CD8⁻ T-cells. Samples were analyzed if the CD8⁺ or CD8⁻ sub-population represented at least 15% of the total sample.

Results

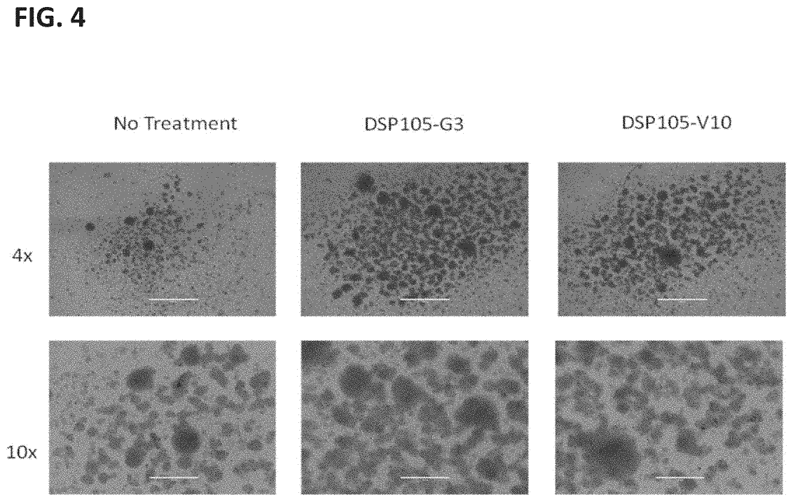

Evaluation of cell growth demonstrated that addition of either the PD1-4-1BBL fusion protein (SEQ ID NO: 49 or 74) or the SIRPα-4-1BBL fusion protein (SEQ ID NO: 89) resulted in expansion of T-cell clusters (Representative images are shown in FIG. 4).

Evaluation of TCR Vβ repertoire demonstrated that addition of either the PD1-4-1BBL fusion protein (SEQ ID NO: 49 or 74), the SIRPα-4-1BBL fusion protein (SEQ ID NO: 89) or the anti-4-1BB antibody resulted in enrichment of specific T-cell families as summarized by the specific families of TCR Vβ frequencies in the two treatment groups for both CD8⁺ T-cells (Table 3 hereinbelow) and CD8− T-cells (Table 4 hereinbelow). In most samples, treatment with the fusion proteins and the antibody resulted in enrichment of few TCR Vβ families mainly in the CD8⁺ T-cell population. Some families appear to be enriched in samples obtained from few patients; for example, Vβ8 was enriched in the CD8⁺ T-cells in 4 samples (OL #1, OL #3, OL #4 and OL #5), in the 2 treatment groups following treatment with either the PD1-4-1BBL or SIRPα-4-1BBL fusion protein; Vβ3 was enriched in the CD8⁺ T-cells in 4 samples (OL #1, OL #2, OL #3 and OL #7), in the 2 treatment groups following treatment with either the PD1-4-1BBL or SIRPα-4-1BBL fusion protein.

TABLE 3
Frequencies (percentages) of TCR Vβ families in CD8⁺ T cells.
OL#1

Group 1 Day 14

| | | PD1- | PD1- | SIRPα- |
|---|---|---|---|---|
| | | 4-1BBL | 4-1BBL | 4-1BBL |
| Vβ | | (SEQ ID | (SEQ ID | (SEQ ID |
| families | Control | NO: 49) | NO: 74) | NO: 89) |
| 1 | 3 | 5 | 2 | 0 |
| 2 | 4 | 3 | 4 | 2 |
| 3 | 0 | 5 | 7 | 12 |
| 4 | 0 | 0 | 0 | 0 |
| 5.1 | 0 | 0 | 0 | 0 |
| 5.2 | 0 | 0 | 0 | 0 |
| 5.3 | 0 | 0 | 0 | 0 |
| 7.1 | 0 | 0 | 0 | 0 |
| 7.2 | 0 | 2 | 2 | 0 |
| 8 | 2 | 7 | 2 | 2 |
| 9 | 2 | 1 | 0 | 0 |
| 11 | 0 | 0 | 0 | 0 |
| 12 | 2 | 2 | 1 | 2 |
| 13.1 | 2 | 1 | 1 | 2 |
| 13.2 | 2 | 0 | 0 | 0 |
| 13.6 | 0 | 1 | 1 | 2 |
| 14 | 6 | 8 | 7 | 7 |
| 16 | 12 | 6 | 11 | 11 |
| 17 | 0 | 1 | 0 | 0 |
| 18 | 0 | 0 | 0 | 0 |
| 20 | 2 | 2 | 2 | 3 |
| 21.3 | 1 | 2 | 1 | 5 |
| 22 | 0 | 0 | 0 | 0 |
| 23 | 11 | 4 | 0 | 0 |

OL# 2

| | | | | | | Group 2 Day 14 | | | |
|---|---|---|---|---|---|---|---|---|---|
| | | Group 1 Day 14 | | | | (treatment from day 5) | | | |
| | | | PD1- | PD1- | SIRPα- | | PD1- | PD1- | SIRPα- |
| | | | 4-1BBL | 4-1BBL | 4-1BBL | | 4-1BBL | 4-1BBL | 4-1BBL |
| Vβ | Day 3 | | (SEQ ID | (SEQ ID | (SEQ ID | | (SEQ ID | (SEQ ID | (SEQ ID |
| family | % | Control | NO: 49) | NO: 74) | NO: 89) | Control | NO: 49) | NO: 74) | NO: 89) |
| 1 | 2 | 0 | 0 | 0 | 0 | 0 | 2 | 0 | 0 |
| V2 | 4 | 0 | 0 | 0 | 4 | 0 | 2 | 0 | 0 |
| 3 | 2 | 0 | 5 | 1 | 3 | 0 | 2 | 5 | 0 |
| 4 | 0 | 0 | 0 | 0 | 4 | 0 | 2 | 0 | 0 |
| V5.1 | 0 | 0 | 0 | 0 | 3 | 0 | 2 | 0 | 0 |
| 5.2 | 0 | 0 | 0 | 0 | 14 | 0 | 0 | 0 | 2 |
| 5.3 | 0 | 0 | 0 | 0 | 1 | 0 | 0 | 0 | 1 |
| 7.1 | 0 | 0 | 0 | 0 | 4 | 0 | 3 | 0 | 0 |
| 7.2 | 2 | 2 | 3 | 2 | 2 | 0 | 1 | 3 | 0 |
| 8 | 6 | 0 | 3 | 3 | 2 | 0 | 1 | 4 | 7 |
| 9 | 0 | 0 | 0 | 0 | 3 | 0 | 0 | 0 | 0 |
| 11 | 0 | 0 | 0 | 0 | 3 | 0 | 0 | 0 | 2 |
| 12 | 14 | 0 | 3 | 5 | 2 | 0 | 1 | 4 | 0 |
| 13.1 | 1 | 0 | 0 | 0 | 3 | 0 | 0 | 0 | 0 |
| 13.2 | 6 | 0 | 0 | 0 | 2 | 0 | 1 | 3 | 0 |
| 13.6 | 1 | 0 | 0 | 0 | 3 | 0 | 2 | 0 | 69 |
| 14 | 9 | 0 | 3 | 2 | 1 | 0 | 1 | 3 | 0 |
| 16 | 2 | 1 | 3 | 3 | 2 | 0 | 1 | 4 | 0 |
| 17 | 0 | 0 | 0 | 0 | 2 | 0 | 2 | 0 | 0 |
| 18 | 0 | 0 | 0 | 0 | 3 | 0 | 0 | 0 | 2 |
| 20 | 5 | 0 | 3 | 3 | 2 | 0 | 0 | 4 | 0 |
| 21.3 | 2 | 0 | 3 | 2 | 2 | 0 | 1 | 4 | 0 |
| 22 | 0 | 0 | 0 | 0 | 5 | 0 | 2 | 0 | 0 |
| 23 | 0 | 0 | 0 | 0 | 3 | 0 | 0 | 0 | 1 |

OL# 3

Group 1 Day 14

| | | PD1- | PD1- | SIRPα- |
|---|---|---|---|---|
| | | 4-1BBL | 4-1BBL | 4-1BBL |
| Vβ | | (SEQ ID | (SEQ ID | (SEQ ID |
| family | Control | NO: 49) | NO: 74) | NO: 89) |
| 1 | 0 | 2 | 0 | 0 |
| 2 | 0 | 0 | 0 | 0 |
| 3 | 3 | 8 | 4 | 3 |
| V4 | 0 | 0 | 0 | 0 |
| 5.1 | 0 | 0 | 0 | 0 |
| 5.2 | 7 | 6 | 9 | 5 |
| 5.3 | 0 | 0 | 0 | 0 |
| 7.1 | 0 | 0 | 0 | 0 |
| 7.2 | 0 | 0 | 0 | 0 |
| 8 | 3 | 7 | 4 | 20 |
| 9 | 0 | 0 | 0 | 0 |
| 11 | 0 | 0 | 0 | 0 |
| 12 | 0 | 1 | 1 | 1 |
| 13.1 | 3 | 1 | 0 | 0 |
| 13.2 | 0 | 2 | 0 | 1 |
| 13.6 | 2 | 2 | 2 | 2 |
| 14 | 3 | 7 | 2 | 3 |
| 16 | 2 | 2 | 3 | 2 |
| 17 | 0 | 0 | 0 | 0 |
| 18 | 0 | 0 | 0 | 0 |
| 20 | 2 | 3 | 1 | 2 |
| 21.3 | 38 | 41 | 29 | 45 |
| 22 | 0 | 0 | 0 | 0 |
| 23 | 1 | 2 | 0 | 0 |

OL#4

| | | Group 1 Day 15 | | | | Group 2 Day 15 | | | |
|---|---|---|---|---|---|---|---|---|---|
| | | | PD1- | PD1- | SIRPα- | | PD1- | PD1- | SIRPα- |
| | | | 4-1BBL | 4-1BBL | 4-1BBL | | 4-1BBL | 4-1BBL | 4-1BBL |
| Vβ | Day 3 | | (SEQ ID | (SEQ ID | (SEQ ID | | (SEQ ID | (SEQ ID | (SEQ ID |
| family | % | Control | NO: 49) | NO: 74) | NO: 89) | Control | NO: 49) | NO: 74) | NO: 89) |
| 1 | 9 | 8 | 8 | 7 | 8 | 7 | 8 | 3 | 6 |
| 2 | 3 | 0 | 3 | 3 | 3 | 2 | 2 | 2 | 2 |
| 3 | 2 | 2 | 3 | 3 | 2 | 1 | 2 | 1 | 2 |
| 4 | 9 | 0 | 0 | 0 | 0 | 0 | 0 | 0 | 0 |
| 5.1 | 9 | 0 | 0 | 1 | 1 | 0 | 1 | 2 | 3 |
| 5.2 | 0 | 0 | 0 | 0 | 0 | 0 | 0 | 0 | 0 |
| 5.3 | 1 | 2 | 0 | 1 | 0 | 1 | 2 | 1 | 0 |
| 7.1 | 11 | 0 | 0 | 1 | 0 | 0 | 1 | 0 | 0 |
| 7.2 | 2 | 0 | 0 | 0 | 0 | 0 | 0 | 0 | 0 |
| 8 | 10 | 4 | 11 | 8 | 7 | 4 | 7 | 3 | 8 |
| 9 | 7 | 0 | 1 | 0 | 0 | 0 | 0 | 0 | 0 |
| 11 | 0 | 0 | 0 | 0 | 0 | 0 | 0 | 0 | 0 |
| 12 | 12 | 1 | 0 | 0 | 2 | 0 | 1 | 1 | 1 |
| 13.1 | 32 | 21 | 13 | 16 | 17 | 17 | 11 | 22 | 19 |
| 13.2 | 1 | 1 | 0 | 2 | 3 | 3 | 0 | 2 | 3 |
| 13.6 | 9 | 11 | 12 | 11 | 11 | 7 | 9 | 8 | 12 |
| 14 | 11 | 3 | 3 | 4 | 3 | 3 | 3 | 4 | 6 |
| 16 | 7 | 12 | 15 | 27 | 29 | 8 | 13 | 15 | 12 |
| 17 | 9 | 0 | 0 | 0 | 0 | 0 | 0 | 0 | 1 |
| 18 | 0 | 0 | 0 | 0 | 0 | 0 | 0 | 0 | 0 |
| 20 | 3 | 16 | 8 | 17 | 9 | 7 | 0 | 18 | 21 |
| 21.3 | 4 | 1 | 2 | 1 | 2 | 2 | 0 | 3 | 4 |
| 22 | 4 | 0 | 0 | 0 | 0 | 0 | 0 | 0 | 0 |
| 23 | 0 | 0 | 1 | 0 | 0 | 0 | 0 | 0 | 0 |

OL#5

Group 1 Day 14

| | | | PD1- | PD1- | SIRPα- |
|---|---|---|---|---|---|
| | | | 4-1BBL | 4-1BBL | 4-1BBL |
| Vβ | Day 3 | | (SEQ ID | (SEQ ID | (SEQ ID |
| family | % | Control | NO: 49) | NO: 4) | NO: 89) |
| 1 | 14 | 51 | 45 | 52 | 38 |
| 2 | 2 | 0 | 0 | 0 | 0 |
| 3 | 2 | 0 | 1 | 0 | 0 |
| 4 | 0 | 0 | 0 | 0 | 0 |
| 5.1 | 6 | 0 | 2 | 3 | 7 |
| 5.2 | 0 | 0 | 0 | 0 | 0 |
| 5.3 | 1 | 0 | 0 | 0 | 0 |
| 7.1 | 2 | 0 | 1 | 0 | 0 |
| 7.2 | 0 | 0 | 0 | 0 | 0 |
| 8 | 4 | 10 | 19 | 18 | 21 |
| 9 | 0 | 0 | 0 | 0 | 0 |
| 11 | 0 | 0 | 0 | 0 | 0 |
| 12 | 0 | 0 | 0 | 0 | 0 |
| 13.1 | 0 | 0 | 0 | 0 | 0 |
| 13.2 | 2 | 0 | 0 | 1 | 0 |
| 13.6 | 0 | 0 | 0 | 1 | 1 |
| 14 | 3 | 5 | 4 | 3 | 9 |
| 16 | 2 | 17 | 14 | 16 | 17 |
| 17 | 16 | 0 | 0 | 0 | 0 |
| 18 | 2 | 0 | 0 | 0 | 1 |
| 20 | 3 | 0 | 0 | 0 | 2 |
| 21.3 | 0 | 0 | 0 | 0 | 0 |
| 22 | 4 | 0 | 0 | 0 | 0 |
| 23 | 0 | 0 | 0 | 0 | 1 |

OL#7

| | Group 1 Day 15 | | | | Group 2 Day 15 | | | |
|---|---|---|---|---|---|---|---|---|
| | | PD1- | PD1- | SIRPα- | | PD1- | PD1- | SIRPα- |
| | | 4-1BBL | 4-1BBL | 4-1BBL | | 4-1BBL | 4-1BBL | 4-1BBL |
| Vβ | | (SEQ ID | (SEQ ID | (SEQ ID | | (SEQ ID | (SEQ ID | (SEQ ID |
| family | Control | NO: 49) | NO: 74) | NO: 89) | Control | NO: 49) | NO: 74) | NO: 89) |
| 1 | 63 | 57 | 52 | 58 | 58 | 63 | 61 | 62 |
| 2 | 4 | 0 | 2 | 3 | 5 | 3 | 4 | 4 |
| 3 | 1 | 7 | 4 | 1 | 1 | 0 | 0 | 1 |
| 4 | 0 | 2 | 0 | 1 | 0 | 0 | 0 | 0 |
| 5.1 | 7 | 5 | 17 | 10 | 2 | 3 | 3 | 5 |
| 5.2 | 0 | 0 | 0 | 0 | 0 | 0 | 0 | 0 |
| 5.3 | 0 | 0 | 0 | 0 | 2 | 0 | 2 | 0 |
| 7.1 | 5 | 7 | 13 | 7 | 7 | 6 | 5 | 7 |
| 7.2 | 1 | 0 | 1 | 0 | 0 | 0 | 0 | 0 |
| 8 | 0 | 0 | 0 | 2 | 0 | 0 | 0 | 0 |
| 9 | 0 | 0 | 0 | 0 | 0 | 0 | 0 | 0 |
| 11 | 0 | 0 | 0 | 0 | 0 | 0 | 0 | 0 |
| 12 | 0 | 0 | 0 | 0 | 0 | 0 | 0 | 0 |
| 13.1 | 0 | 0 | 2 | 0 | 0 | 0 | 0 | 0 |
| 13.2 | 0 | 7 | 8 | 7 | 5 | 7 | 10 | 12 |
| 13.6 | 0 | 0 | 2 | 1 | 0 | 0 | 0 | 0 |
| 14 | 8 | 8 | 3 | 4 | 8 | 9 | 11 | 8 |
| 16 | 2 | 2 | 3 | 4 | 1 | 1 | 0 | 2 |
| 17 | 0 | 0 | 0 | 0 | 5 | 4 | 5 | 6 |
| 18 | 1 | 1 | 0 | 0 | 0 | 0 | 0 | 0 |
| 20 | 3 | 3 | 5 | 3 | 7 | 8 | 8 | 10 |
| 21.3 | 2 | 0 | 0 | 0 | 0 | 1 | 0 | 0 |
| 22 | 0 | 0 | 1 | 0 | 0 | 0 | 0 | 0 |
| 23 | 0 | 0 | 0 | 1 | 0 | 1 | 0 | 2 |

OL#8

Group 1 Day 14

| | | | PD1- | PD1- | SIRPα- |
|---|---|---|---|---|---|
| | | | 4-1BBL | 4-1BBL | 4-1BBL |
| Vβ | Day 3 | | (SEQ ID | (SEQ ID | (SEQ ID |
| family | Control | Control | NO: 49) | NO: 74) | NO: 89) |
| 1 | 6 | 3 | 3 | 2 | 3 |
| 2 | 10 | 19 | 18 | 21 | 18 |
| 3 | 14 | 14 | 14 | 14 | 13 |
| 4 | 0 | 0 | 0 | 0 | 0 |
| 5.1 | 0 | 1 | 0 | 0 | 0 |
| 5.2 | 1 | 0 | 0 | 0 | 0 |
| 5.3 | 0 | 0 | 0 | 0 | 0 |
| 7.1 | 3 | 2 | 2 | 3 | 1 |
| 7.2 | 1 | 0 | 0 | 0 | 0 |
| 8 | 4 | 9 | 11 | 8 | 5 |
| 9 | 0 | 0 | 0 | 0 | 0 |
| 11 | 0 | 0 | 0 | 0 | 0 |
| 12 | 0 | 0 | 0 | 0 | 0 |
| 13.1 | 1 | 0 | 0 | 0 | 0 |
| 13.2 | 1 | 2 | 2 | 1 | 2 |
| 13.6 | 0 | 0 | 0 | 0 | 0 |
| 14 | 4 | 2 | 2 | 2 | 1 |
| 16 | 7 | 3 | 4 | 3 | 2 |
| 17 | 0 | 0 | 1 | 0 | 0 |
| 18 | 0 | 0 | 0 | 0 | 0 |
| 20 | 11 | 10 | 14 | 13 | 11 |
| 21.3 | 0 | 2 | 2 | 2 | 3 |
| 22 | 0 | 0 | 0 | 0 | 0 |
| 23 | 5 | 6 | 9 | 5 | 7 |

OL#9

| | | Group 1 Day 16 | | | | Group 2 Day 16 | | | |
|---|---|---|---|---|---|---|---|---|---|
| | | | PD1- | PD1- | SIRPα- | | PD1- | PD1- | SIRPα- |
| | | | 4-1BBL | 4-1BBL | 4-1BBL | | 4-1BBL | 4-1BBL | 4-1BBL |
| Vβ | Day 3 | | (SEQ ID | (SEQ ID | (SEQ ID | | (SEQ ID | (SEQ ID | (SEQ ID |
| family | % | Control | NO: 49) | NO: 74) | NO: 89) | Control | NO: 49) | NO: 74) | NO: 89) |
| 1 | 8 | 5 | 3 | 3 | 4 | 0 | 3 | 11 | 0 |
| 2 | 0 | 0 | 0 | 0 | 0 | 0 | 0 | 0 | 0 |
| 3 | 5 | 2 | 0 | 1 | 2 | 3 | 4 | 5 | 4 |
| 4 | 0 | 0 | 0 | 0 | 0 | 0 | 0 | 0 | 0 |
| 5.1 | 2 | 0 | 0 | 0 | 0 | 0 | 1 | 1 | 0 |
| 5.2 | 0 | 0 | 0 | 0 | 0 | 0 | 0 | 0 | 0 |
| 5.3 | 5 | 7 | 1 | 1 | 3 | 3 | 6 | 6 | 8 |
| 7.1 | 6 | 0 | 0 | 0 | 0 | 0 | 0 | 0 | 0 |
| 7.2 | 0 | 0 | 0 | 0 | 0 | 0 | 0 | 4 | 0 |
| 8 | 4 | 0 | 0 | 0 | 0 | 1 | 1 | 2 | 1 |
| Vb9 | 0 | 0 | 0 | 0 | 0 | 0 | 0 | 0 | 0 |
| 11 | 0 | 0 | 0 | 0 | 0 | 0 | 0 | 0 | 0 |
| 12 | 0 | 0 | 0 | 0 | 0 | 0 | 0 | 0 | 0 |
| 13 | 27 | 48 | 43 | 44 | 33 | 39 | 38 | 30 | 43 |
| 13.2 | 6 | 2 | 3 | 2 | 2 | 0 | 1 | 0 | 1 |
| 13.6 | 1 | 2 | 1 | 2 | 2 | 1 | 3 | 1 | 2 |
| 14 | 5 | 2 | 0 | 0 | 0 | 1 | 2 | 1 | 1 |
| 16 | 5 | 8 | 3 | 9 | 12 | 27 | 15 | 0 | 16 |
| 17 | 0 | 0 | 0 | 0 | 0 | 0 | 0 | 0 | 0 |
| 18 | 0 | 0 | 0 | 0 | 0 | 0 | 0 | 0 | 0 |
| 20 | 3 | 1 | 0 | 0 | 2 | 1 | 2 | 2 | 0 |
| 21.3 | 1 | 0 | 0 | 0 | 0 | 0 | 0 | 0 | 0 |
| 22 | 0 | 0 | 0 | 0 | 0 | 0 | 0 | 0 | 0 |
| 23 | 0 | 0 | 0 | 0 | 0 | 0 | 0 | 0 | 0 |

AML#6

Group 1 Day 14

| | | | PD1- | PD1- | SIRPα- |
|---|---|---|---|---|---|
| | | | 4-1BBL | 4-1BBL | 4-1BBL |
| Vβ | Day 3 | | (SEQ ID | (SEQ ID | (SEQ ID |
| family | % | Control | NO: 49) | NO: 74) | NO: 89) |
| 1 | 5 | 0 | 0 | 0 | 0 |
| 2 | 8 | 7 | 3 | 3 | 4 |
| 3 | 10 | 10 | 3 | 5 | 5 |
| 4 | 0 | 0 | 0 | 0 | 0 |
| 5.1 | 4 | 0 | 0 | 0 | 0 |
| 5.2 | 0 | 0 | 0 | 0 | 0 |
| 5.3 | 0 | 0 | 3 | 0 | 0 |
| 7.1 | 3 | 1 | 8 | 0 | 0 |
| 7.2 | 2 | 3 | 1 | 0 | 4 |
| 8 | 4 | 6 | 2 | 4 | 3 |
| 9 | 2 | 0 | 0 | 0 | 0 |
| 11 | 0 | 0 | 0 | 0 | 0 |
| 12 | 0 | 0 | 0 | 0 | 0 |
| 13.1 | 3 | 2 | 1 | 0 | 2 |
| 13.2 | 5 | 1 | 1 | 0 | 0 |
| 13.6 | 3 | 0 | 0 | 0 | 0 |
| 14 | 5 | 7 | 16 | 10 | 7 |
| 16 | 4 | 5 | 1 | 2 | 2 |
| 17 | 0 | 0 | 0 | 0 | 0 |
| 18 | 0 | 0 | 0 | 0 | 0 |
| 20 | 6 | 7 | 3 | 3 | 4 |
| 21.3 | 2 | 14 | 17 | 11 | 10 |
| 22 | 0 | 0 | 0 | 0 | 0 |
| 23 | 2 | 0 | 0 | 0 | 0 |

OL#5

Group 1 Day 14
anti-4-1BB antibody (μg/mL)

| Vβ family | 0 | 0.5 | 2 | 5 |
|---|---|---|---|---|
| 1 | 51 | 17 | 31 | 21 |
| 2 | 0 | 1 | 1 | 0 |
| 3 | 0 | 1 | 2 | 2 |
| 4 | 0 | 0 | 0 | 0 |
| 5.1 | 0 | 5 | 4 | 0 |
| 5.2 | 0 | 0 | 0 | 0 |
| 5.3 | 0 | 0 | 2 | 0 |
| 7.1 | 0 | 1 | 2 | 0 |
| 7.2 | 0 | 0 | 0 | 0 |
| 8 | 10 | 11 | 8 | 12 |
| 9 | 0 | 0 | 0 | 0 |
| 11 | 0 | 0 | 0 | 0 |
| 12 | 0 | 0 | 0 | 0 |
| 13.1 | 0 | 0 | 0 | 0 |
| 13.2 | 0 | 3 | 8 | 4 |
| 13.6 | 0 | 2 | 2 | 1 |
| 14 | 5 | 7 | 4 | 8 |
| 16 | 17 | 22 | 13 | 21 |
| 17 | 0 | 1 | 0 | 0 |
| 18 | 0 | 0 | 0 | 0 |
| 20 | 0 | 6 | 4 | 15 |
| 21.3 | 0 | 0 | 0 | 0 |
| 22 | 0 | 0 | 0 | 0 |
| 23 | 0 | 1 | 0 | 1 |

OL#8

| | Group 1 anti-4-1BB antibody | | |
|---|---|---|---|
| | (μg/ml) Day 14 | | |
| Vβ family | Control | 0.5 | 2 |
| 1 | 3 | 3 | 2 |
| 2 | 19 | 19 | 16 |
| 3 | 14 | 15 | 12 |
| 4 | 0 | 0 | 0 |
| 5.1 | 1 | 1 | 0 |
| 5.2 | 0 | 0 | 0 |
| 5.3 | 0 | 0 | 0 |
| 7.1 | 2 | 1 | 1 |
| 7.2 | 0 | 0 | 0 |
| 8 | 9 | 7 | 5 |
| 9 | 0 | 0 | 0 |
| 11 | 0 | 0 | 0 |
| 12 | 0 | 0 | 0 |
| 13.1 | 0 | 0 | 0 |
| 13.2 | 2 | 1 | 1 |
| 13.6 | 0 | 0 | 0 |
| 14 | 2 | 2 | 1 |
| 16 | 3 | 3 | 2 |
| 17 | 0 | 0 | 0 |
| 18 | 0 | 0 | 0 |
| 20 | 10 | 11 | 14 |
| 21.3 | 2 | 2 | 4 |
| 22 | 0 | 0 | 0 |
| 23 | 6 | 10 | 14 |

TABLE 4
Frequencies (percentages) of TCR Vβ families in CD8⁻ T cells
OL#1

Group 1 Day 14

| | | | PD1- | PD1- | SIRPα- |
|---|---|---|---|---|---|
| | | | 4-1BBL | 4-1BBL | 4-1BBL |
| Vβ | Day 2 | | (SEQ ID | (SEQ ID | (SEQ ID |
| family | % | Control | NO: 49) | NO: 74) | NO: 89) |
| 1 | 0 | 1 | 0 | 2 | 1 |
| 2 | 1 | 0 | 0 | 1 | 1 |
| 3 | 8 | 2 | 0 | 0 | 0 |
| 4 | 0 | 0 | 0 | 1 | 1 |
| 5.1 | 1 | 4 | 2 | 3 | 11 |
| 5.2 | 0 | 5 | 2 | 2 | 4 |
| 5.3 | 0 | 0 | 0 | 0 | 0 |
| 7.1 | 0 | 2 | 0 | 2 | 2 |
| 7.2 | 3 | 0 | 0 | 2 | 1 |
| 8 | 6 | 2 | 0 | 0 | 1 |
| 9 | 0 | 0 | 0 | 0 | 0 |
| 11 | 0 | 0 | 0 | 0 | 1 |
| 12 | 4 | 0 | 0 | 0 | 0 |
| 13.1 | 0 | 1 | 0 | 0 | 1 |
| 13.2 | 0 | 0 | 0 | 0 | 0 |
| 13.6 | 0 | 1 | 2 | 3 | 9 |
| 14 | 2 | 0 | 0 | 0 | 0 |
| 16 | 1 | 2 | 1 | 1 | 1 |
| 17 | 0 | 5 | 2 | 3 | 6 |
| 18 | 0 | 0 | 0 | 0 | 0 |
| V20 | 4 | 0 | 0 | 0 | 0 |
| 21.3 | 6 | 0 | 0 | 0 | 0 |
| 22 | 0 | 1 | 1 | 3 | 6 |
| 23 | 0 | 0 | 1 | 1 | 2 |

OL#2

| | | | | | | Group 2 Day 14 | | | |
|---|---|---|---|---|---|---|---|---|---|
| | | Group 1 Day 14 | | | | (treatment from day 5) | | | |
| | | | PD1- | PD1- | SIRPα- | PD1- | PD1- | SIRPα- | PD1- |
| | | | 4-1BBL | 4-1BBL | 4-1BBL | 4-1BBL | 4-1BBL | 4-1BBL | 4-1BBL |
| Vβ | Day 3 | | (SEQ ID | (SEQ ID | (SEQ ID | (SEQ ID | (SEQ ID | (SEQ ID | (SEQ ID |
| family | % | Control | NO: 49) | NO: 74) | NO: 89) | NO: 49) | NO: 74) | NO: 89) | NO: 49) |
| 1 | 0 | 0 | 0 | 0 | 1 | 0 | 0 | 0 | 0 |
| 2 | 3 | 0 | 0 | 0 | 0 | 0 | 2 | 0 | 0 |
| 3 | 0 | 0 | 17 | 4 | 0 | 0 | 17 | 0 | 0 |
| 4 | 0 | 0 | 0 | 0 | 2 | 0 | 0 | 0 | 0 |
| 5.1 | 0 | 0 | 0 | 0 | 0 | 0 | 0 | 0 | 0 |
| 5.2 | 0 | 0 | 0 | 0 | 14 | 0 | 0 | 0 | 0 |
| 5.3 | 0 | 0 | 0 | 0 | 9 | 0 | 0 | 0 | 0 |
| 7.1 | 0 | 0 | 0 | 0 | 5 | 0 | 0 | 0 | 0 |
| 7.2 | 0 | 0 | 16 | 2 | 0 | 0 | 19 | 0 | 0 |
| 8 | 2 | 0 | 14 | 2 | 0 | 0 | 13 | 0 | 2 |
| 9 | 0 | 0 | 0 | 0 | 10 | 0 | 0 | 0 | 0 |
| 11 | 0 | 0 | 0 | 0 | 13 | 0 | 0 | 0 | 0 |
| 12 | 0 | 0 | 15 | 2 | 0 | 0 | 16 | 0 | 0 |
| 13.1 | 1 | 0 | 0 | 0 | 3 | 0 | 0 | 0 | 3 |
| 13.2 | 1 | 0 | 0 | 0 | 17 | 0 | 0 | 0 | 0 |
| 13.6 | 0 | 0 | 0 | 0 | 2 | 0 | 0 | 0 | 77 |
| 14 | 2 | 0 | 17 | 2 | 0 | 0 | 16 | 0 | 0 |
| 16 | 3 | 0 | 12 | 3 | 0 | 0 | 12 | 0 | 0 |
| 17 | 0 | 0 | 0 | 0 | 0 | 0 | 0 | 0 | 0 |
| 18 | 0 | 0 | 0 | 0 | 9 | 0 | 0 | 0 | 2 |
| 20 | 2 | 0 | 14 | 2 | 0 | 0 | 15 | 0 | 0 |
| 21.3 | 4 | 0 | 17 | 2 | 0 | 0 | 14 | 0 | 0 |
| 22 | 0 | 0 | 0 | 0 | 2 | 1 | 0 | 0 | 0 |
| 23 | 0 | 0 | 0 | 0 | 1 | 0 | 0 | 0 | 0 |

OL#3

Group 1 Day 14

| | | | PD1- | PD1- | SIRPα- |
|---|---|---|---|---|---|
| | | | 4-1BBL | 4-1BBL | 4-1BBL |
| Vβ | Day 3 | | (SEQ ID | (SEQ ID | (SEQ ID |
| family | % | Control | NO: 49) | NO: 74) | NO: 89) |
| 1 | 0 | 2 | 0 | 0 | 0 |
| 2 | 0 | 1 | 0 | 0 | 2 |
| 3 | 0 | 2 | 3 | 2 | 4 |
| 4 | 0 | 0 | 0 | 0 | 0 |
| 5.1 | 2 | 0 | 0 | 0 | 0 |
| 5.2 | 1 | 0 | 0 | 0 | 0 |
| 5.3 | 0 | 0 | 0 | 0 | 0 |
| 7.1 | 0 | 0 | 0 | 0 | 0 |
| 7.2 | 0 | 0 | 0 | 0 | 0 |
| 8 | 0 | 2 | 3 | 1 | 2 |
| 9 | 0 | 0 | 0 | 0 | 0 |
| 11 | 0 | 0 | 0 | 0 | 0 |
| 12 | 0 | 0 | 0 | 0 | 0 |
| 13.1 | 0 | 0 | 0 | 0 | 0 |
| 13.2 | 0 | 3 | 4 | 1 | 4 |
| 13.6 | 1 | 0 | 0 | 0 | 0 |
| 14 | 0 | 0 | 2 | 0 | 2 |
| 16 | 0 | 0 | 1 | 0 | 1 |
| 17 | 0 | 0 | 0 | 0 | 0 |
| 18 | 0 | 0 | 0 | 0 | 0 |
| 20 | 0 | 9 | 17 | 11 | 12 |
| 21.3 | 0 | 2 | 1 | 1 | 2 |
| 22 | 0 | 0 | 0 | 0 | 0 |
| 23 | 0 | 4 | 10 | 1 | 5 |

OL#4

| | |
|---|---|
| Group 1 Day 14 | Group 2 Day 14 |

| | | | PD1- | PD1- | SIRPα- | | PD1- | PD1- | SIRPα- |
|---|---|---|---|---|---|---|---|---|---|
| | | | 4-1BBL | 4-1BBL | 4-1BBL | | 4-1BBL | 4-1BBL | 4-1BBL |
| Vβ | Day 3 | | (SEQ ID | (SEQ ID | (SEQ ID | | (SEQ ID | (SEQ ID | (SEQ ID |
| family | % | Control | NO: 49) | NO: 74) | NO: 89) | Control | NO: 49) | NO: 74) | NO: 89) |
| 1 | 0 | 2 | 0 | 0 | 2 | 1 | 1 | 0 | 0 |
| 2 | 3 | 11 | 5 | 5 | 4 | 4 | 6 | 5 | 5 |
| 3 | 38 | 5 | 3 | 2 | 1 | 2 | 2 | 1 | 2 |
| 4 | 1 | 0 | 0 | 0 | 0 | 0 | 0 | 0 | 0 |
| 5.1 | 0 | 0 | 0 | 0 | 0 | 0 | 0 | 0 | 0 |
| 5.2 | 0 | 0 | 0 | 0 | 0 | 0 | 0 | 0 | 0 |
| 5.3 | 0 | 0 | 0 | 1 | 0 | 0 | 0 | 1 | 0 |
| 7.1 | 0 | 2 | 1 | 3 | 0 | 0 | 2 | 0 | 0 |
| 7.2 | 35 | 0 | 0 | 0 | 0 | 0 | 1 | 0 | 1 |
| 8 | 13 | 2 | 3 | 4 | 2 | 0 | 4 | 9 | 3 |
| 9 | 0 | 1 | 2 | 1 | 0 | 2 | 2 | 0 | 0 |
| 11 | 0 | 0 | 0 | 0 | 0 | 0 | 0 | 0 | 0 |
| 12 | 38 | 1 | 2 | 0 | 0 | 0 | 1 | 0 | 2 |
| 13.1 | 2 | 1 | 2 | 1 | 1 | 0 | 1 | 0 | 1 |
| 13.2 | 0 | 1 | 1 | 0 | 1 | 0 | 0 | 0 | 1 |
| 13.6 | 0 | 1 | 0 | 0 | 0 | 0 | 0 | 0 | 0 |
| 14 | 1 | 4 | 2 | 4 | 4 | 6 | 4 | 3 | 7 |
| 16 | 3 | 5 | 5 | 5 | 11 | 5 | 4 | 7 | 6 |
| 17 | 0 | 0 | 0 | 0 | 0 | 0 | 0 | 0 | 0 |
| 18 | 0 | 0 | 0 | 0 | 0 | 0 | 0 | 0 | 0 |
| 20 | 42 | 7 | 10 | 7 | 4 | 8 | 7 | 17 | 12 |
| 21.3 | 37 | 2 | 2 | 2 | 1 | 2 | 6 | 2 | 2 |
| 22 | 0 | 0 | 0 | 0 | 0 | 0 | 0 | 0 | 0 |
| 23 | 0 | 1 | 0 | 0 | 0 | 0 | 0 | 0 | 0 |

OL#7

| | |
|---|---|
| Group 1 Day 15 | Group 2 Day 15 |

| | | | PD1- | PD1- | SIRPα- | | PD1- | PD1- | SIRPα- |
|---|---|---|---|---|---|---|---|---|---|
| | | | 4-1BBL | 4-1BBL | 4-1BBL | | 4-1BBL | 4-1BBL | 4-1BBL |
| Vβ | Day 3 | | (SEQ ID | (SEQ ID | (SEQ ID | | (SEQ ID | (SEQ ID | (SEQ ID |
| family | % | Control | NO: 49) | NO: 74) | NO: 89) | Control | NO: 49) | NO: 74) | NO: 89) |
| 1 | 0 | 2 | 1 | 2 | 1 | 2 | 2 | 2 | 2 |
| 2 | 3 | 15 | 5 | 5 | 17 | 16 | 13 | 18 | 16 |
| 3 | 3 | 3 | 3 | 3 | 3 | 3 | 4 | 3 | 3 |
| 4 | 0 | 0 | 2 | 2 | 1 | 2 | 1 | 1 | 2 |
| 5.1 | 0 | 4 | 2 | 0 | 0 | 3 | 3 | 3 | 4 |
| 5.2 | 0 | 0 | 0 | 0 | 0 | 0 | 1 | 0 | 0 |
| 5.3 | 0 | 2 | 0 | 0 | 0 | 0 | 1 | 0 | 0 |
| 7.1 | 0 | 0 | 0 | 0 | 0 | 0 | 0 | 0 | 0 |
| 7.2 | 0 | 2 | 1 | 1 | 1 | 1 | 1 | 1 | 0 |
| 8 | 3 | 3 | 4 | 5 | 5 | 4 | 2 | 5 | 3 |
| 9 | 0 | 1 | 1 | 0 | 0 | 0 | 0 | 0 | 1 |
| 11 | 0 | 0 | 0 | 0 | 0 | 0 | 0 | 0 | 0 |
| 12 | 1 | 0 | 1 | 0 | 0 | 0 | 0 | 0 | 1 |
| 13.1 | 0 | 7 | 2 | 2 | 2 | 3 | 6 | 7 | 6 |
| 13.2 | 0 | 0 | 10 | 11 | 5 | 6 | 7 | 8 | 11 |
| 13.6 | 0 | 4 | 1 | 2 | 3 | 3 | 2 | 3 | 2 |
| 14 | 2 | 8 | 8 | 6 | 18 | 10 | 9 | 7 | 11 |
| 16 | 2 | 4 | 4 | 4 | 5 | 5 | 3 | 3 | 5 |
| 17 | 0 | 0 | 0 | 0 | 0 | 0 | 0 | 0 | 0 |
| 18 | 0 | 0 | 0 | 0 | 0 | 0 | 0 | 0 | 0 |
| 20 | 6 | 3 | 3 | 5 | 5 | 6 | 5 | 5 | 9 |
| 21.3 | 0 | 4 | 1 | 1 | 3 | 4 | 5 | 5 | 4 |
| 22 | 0 | 1 | 0 | 1 | 2 | 2 | 2 | 1 | 2 |
| 23 | 0 | 0 | 0 | 0 | 0 | 2 | 0 | 1 | 3 |

AML#6

Group 1 Day 14

| | | | PD1- | PD1- | SIRPα- |
|---|---|---|---|---|---|
| | | | 4-1BBL | 4-1BBL | 4-1BBL |
| Vβ | Day 3 | | (SEQ ID | (SEQ ID | (SEQ ID |
| family | % | Control | NO: 49) | NO: 74) | NO: 89) |
| 1 | 0 | 0 | 0 | 0 | 0 |
| 2 | 1 | 3 | 0 | 0 | 0 |
| 3 | 3 | 3 | 0 | 0 | 1 |
| 4 | 0 | 0 | 0 | 0 | 0 |
| 5.1 | 0 | 0 | 0 | 0 | 0 |
| 5.2 | 0 | 0 | 0 | 0 | 0 |
| 5.3 | 0 | 0 | 0 | 0 | 0 |
| 7.1 | 0 | 0 | 4 | 0 | 0 |
| 7.2 | 0 | 0 | 0 | 0 | 0 |
| 8 | 6 | 0 | 0 | 0 | 2 |
| 9 | 1 | 0 | 0 | 0 | 0 |
| 11 | 0 | 0 | 0 | 0 | 0 |
| 12 | 0 | 1 | 0 | 0 | 0 |
| 13.1 | 0 | 0 | 0 | 0 | 0 |
| 13.2 | 0 | 0 | 0 | 0 | 0 |
| 13.6 | 0 | 0 | 0 | 0 | 0 |
| 14 | 0 | 2 | 0 | 0 | 0 |
| 16 | 1 | 2 | 0 | 0 | 1 |
| 17 | 0 | 0 | 0 | 0 | 0 |
| 18 | 0 | 0 | 0 | 0 | 0 |
| 20 | 12 | 5 | 3 | 2 | 4 |
| 21.3 | 6 | 3 | 0 | 0 | 1 |
| 22 | 0 | 0 | 0 | 0 | 0 |
| 23 | 0 | 0 | 0 | 5 | 0 |

OL#5

Group 1 Day 14
Anti-4-1BB antibody (μg/mL)

| Vβ family | 0 | 0.5 | 2 | 5 |
|---|---|---|---|---|
| 1 | 0 | 0 | 0 | 0 |
| 2 | 3 | 2 | 2 | 2 |
| 3 | 2 | 0 | 0 | 1 |
| 4 | 0 | 0 | 0 | 0 |
| 5.1 | 0 | 0 | 0 | 0 |
| 5.2 | 0 | 0 | 0 | 0 |
| 5.3 | 0 | 0 | 0 | 0 |
| 7.1 | 0 | 0 | 0 | 0 |
| 7.2 | 0 | 0 | 0 | 0 |
| 8 | 0 | 2 | 1 | 1 |
| 9 | 0 | 0 | 0 | 0 |
| 11 | 0 | 0 | 0 | 0 |
| 12 | 1 | 0 | 0 | 1 |
| 13.1 | 0 | 0 | 0 | 0 |
| 13.2 | 0 | 0 | 0 | 0 |
| 13.6 | 0 | 0 | 0 | 0 |
| 14 | 2 | 2 | 1 | 2 |
| 16 | 3 | 2 | 1 | 2 |
| 17 | 0 | 0 | 0 | 0 |
| 18 | 0 | 0 | 0 | 0 |
| 20 | 1 | 12 | 9 | 8 |
| 21.3 | 0 | 0 | 0 | 1 |
| 22 | 0 | 0 | 0 | 0 |
| 23 | 0 | 0 | 0 | 1 |

OL#8

| | Group 1 anti-4-1BB antibody | | |
|---|---|---|---|
| | (μg/ml) Day 14 | | |
| Vβ family | 0 | 0.5 | 2 |
| 1 | 2 | 0 | 0 |
| 2 | 2 | 1 | 2 |
| 3 | 2 | 1 | 2 |
| 4 | 0 | 0 | 0 |
| 5.1 | 1 | 1 | 0 |
| 5.2 | 0 | 0 | 0 |
| 5.3 | 0 | 0 | 0 |
| 7.1 | 0 | 0 | 0 |
| 7.2 | 0 | 0 | 0 |
| 8 | 2 | 1 | 0 |
| 9 | 1 | 0 | 0 |
| 11 | 3 | 0 | 0 |
| 12 | 0 | 0 | 0 |
| 13.1 | 0 | 0 | 0 |
| 13.2 | 1 | 0 | 0 |
| 13.6 | 0 | 0 | 0 |
| 14 | 2 | 1 | 0 |
| 16 | 2 | 1 | 2 |
| 17 | 0 | 0 | 0 |
| 18 | 2 | 0 | 0 |
| 20 | 6 | 5 | 4 |
| 21.3 | 1 | 0 | 0 |
| 22 | 1 | 0 | 0 |
| 23 | 0 | 1 | 0 |

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. A method of culturing T cells, the method comprising:
(a) adding to immune cells comprising T cells obtained from a subject having a pathology a non-cellular agent capable of binding 4-1BB and activating said 4-1BB signaling pathway; and
(b) culturing said immune cells with said agent for more than 7 days.

2. The method of claim 1, wherein said culturing is effected for more than 10 days.

3. The method of claim 1, wherein said culturing is effected for about 14 days.

4. The method of claim 1, being effected without isolation of 4-1BB positive cells prior to said (a) and/or said (b).

5. The method of claim 1, being effected without isolation of 4-1BB positive cells during and/or following said (b).

6. The method of claim 1, being effected without isolation of said T cells from said immune cells prior to said (a) and/or said (b).

7. The method of claim 1, comprising adding at least one cytokine to said immune cells in step (a) and culturing said immune cells with said cytokine in step (b).

8. The method of claim 1, further comprising pre-culturing said immune cells with at least one cytokine prior to said (a).

9-10. (canceled)

11. The method of claim 8, being effected without isolation of said T cells from said immune cells prior to said pre-culturing.

12. The method of claim 1, being effected without adding to said immune cells a T cell stimulatory agent capable of at least transmitting a primary activating signal to said T cells.

13-24. (canceled)

25. The method of claim 1, wherein said non-cellular agent is an antibody.

26. (canceled)

27. The method of claim 1, wherein said non-cellular agent is a fusion polypeptide.

28-33. (canceled)

34. The method of claim 1, further comprising expanding said T cells following said (b).

35. The method of claim 1, further comprising isolating said T cells from said immune cells following said (b).

36. The method of claim 1, further comprising determining a sequence of a T cell receptor (TCR) expressed by at least one of said T cells following said (b).

37. The method of claim 36, further comprising transducing a T cell with a nucleic acid sequence encoding said TCR.

38. The method of claim 1, further comprising transducing said T cells with a nucleic acid sequence encoding a chimeric antigen receptor (CAR) following said (b).

39. The method of claim 1, comprising adoptively transferring said immune cells and/or said T cells following said (b) to a subject in need thereof.

40. T cells obtainable by the method of claim 1.

41. (canceled)

42. A method of treating a pathology that can benefit from adoptive T cell therapy in a subject in need thereof, the method comprising administering to the subject a therapeutically effective amount of the T cells of claim 40, thereby treating the pathology.

43-48. (canceled)


SEQUENCE LISTING (149 sequences)

SEQ ID NO: 1 - protein, 254 aa, Artificial sequence
  Full length 41BBL
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 2 - protein, 205 aa, Artificial sequence
  ORIGINAL 41BBL EC DOMAIN
ACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 3 - protein, 191 aa, Artificial sequence
  191 aa 14-0 in 4-1BBL DOMAIN
SAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 4 - protein, 197 aa, Artificial sequence
  197 aa 41BBL segment
ARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 5 - protein, 185 aa, Artificial sequence
  185 aa 41BBL segment
LREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 6 - protein, 199 aa, Artificial sequence
  199 aa 41BBL segment
SGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 7 - protein, 184 aa, Artificial sequence
  4-1BBL -21-0  184 aa
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 8 - protein, 182 aa, Artificial sequence
  4-1BBL -23-0 182 aa
GPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 9 - protein, 183 aa, Artificial sequence
  4-1BBL -14-8 183 aa
SAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPA

SEQ ID NO: 10 - protein, 176 aa, Artificial sequence
  4-1BBL -21-8  176 aa
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPA

SEQ ID NO: 11 - protein, 5 aa, Artificial sequence
  linker amino acid sequence
GGGGS

SEQ ID NO: 12 - protein, 20 aa, Artificial sequence
  linker amino acid sequence
  REPEAT (1)..(5)
  REPEAT (6)..(10): may be absent
  REPEAT (11)..(15): may be absent
  REPEAT (16)..(20): may be absent
GGGGSGGGGSGGGGSGGGGS

SEQ ID NO: 13 - protein, 9 aa, Artificial sequence
  linker amino acid sequence
GGGGSGGGG

SEQ ID NO: 14 - protein, 10 aa, Artificial sequence
  linker amino acid sequence
GGGGSGGGGS

SEQ ID NO: 15 - protein, 14 aa, Artificial sequence
  linker amino acid sequence
GGGGSGGGGSGGGG

SEQ ID NO: 16 - protein, 15 aa, Artificial sequence
  linker amino acid sequence
  REPEAT (1)..(5)
  REPEAT (6)..(10): may be absent
  REPEAT (11)..(15): may be absent
EAAAKEAAAKEAAAK

SEQ ID NO: 17 - protein, 27 aa, Artificial sequence
  linker amino acid sequence
  REPEAT (2)..(6)
  REPEAT (7)..(11)
  REPEAT (12)..(16): may be absent
  REPEAT (17)..(21): may be absent
  REPEAT (22)..(26): may be absent
AEAAAKEAAAKEAAAKEAAAKEAAAKA

SEQ ID NO: 18 - protein, 12 aa, Artificial sequence
  linker amino acid sequence
AEAAAKEAAAKA

SEQ ID NO: 19 - protein, 46 aa, Artificial sequence
  linker amino acid sequence
AEAAAKEAAAKEAAAKEAAAKALEAEAAAKEAAAKEAAAKEAAAKA

SEQ ID NO: 20 - protein, 5 aa, Artificial sequence
  linker amino acid sequence
PAPAP

SEQ ID NO: 21 - protein, 18 aa, Artificial sequence
  linker amino acid sequence
KESGSVSSEQLAQFRSLD

SEQ ID NO: 22 - protein, 14 aa, Artificial sequence
  linker amino acid sequence
EGKSSGSGSESKST

SEQ ID NO: 23 - protein, 12 aa, Artificial sequence
  linker amino acid sequence
GSAGSAAGSGEF

SEQ ID NO: 24 - protein, 601 aa, Artificial sequence
  601 aa sc3x4-1BBL (-14-0) internal linker (GGGGS)x2+GGGG
SAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 25 - protein, 288 aa, Artificial sequence
  Full length PD1 with wt sp 288 aa
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 26 - protein, 167 aa, Artificial sequence
  PD1 EC with wt sp DOMAIN 197 aa
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQ

SEQ ID NO: 27 - protein, 122 aa, Artificial sequence
  PD1 EC DOMAIN
DSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTER

SEQ ID NO: 28 - protein, 150 aa, Artificial sequence [sequence omitted]

SEQ ID NO: 29 - protein, 167 aa, Artificial sequence
  PD1 EC with wt sp DOMAIN CYS93>Ser substitution
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDSRFRVTQLPNGRDFHMSVVRARRNDSGTYLCGAISLAPKAQIKESLRAELRVTERRAEVPTAHPSPSPRPAGQFQ

SEQ ID NO: 30 - protein, 150 aa, Artificial sequence
  150 aa ORIGINAL PD1 DOMAIN with CYS93>Ser substitution
PGWFLDSPDRPWNPPTFSPALLVVTEGDNATFTCSFSNTSESFVLNWYRMSPSNQTDKLAAFPEDRSQPGQDSRFRVTQLPNGRDFHMSVVRARRNDSGTYLCGAISLAPKAQIKESLRAELRVTERRAEVPTAHPSPSPRPAGQFQTLV

SEQ ID NO: 31 - protein, 140 aa, Artificial sequence
  140 aa -5-5 in PD1 DOMAIN
DSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQ

SEQ ID NO: 32 - protein, 140 aa, Artificial sequence
  140 aa -5-5 in PD1 DOMAIN with CYS93>Ser substitution
DSPDRPWNPPTFSPALLVVTEGDNATFTCSFSNTSESFVLNWYRMSPSNQTDKLAAFPEDRSQPGQDSRFRVTQLPNGRDFHMSVVRARRNDSGTYLCGAISLAPKAQIKESLRAELRVTERRAEVPTAHPSPSPRPAGQ

SEQ ID NO: 33 - skipped

SEQ ID NO: 34 - protein, 128 aa, Artificial sequence
  128 aa PD1 segment
PWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSP

SEQ ID NO: 35 - protein, 135 aa, Artificial sequence
  135 aa PD1 segment
PWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQ

SEQ ID NO: 36 - protein, 133 aa, Artificial sequence
  133 aa PD1 segment
DSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSP

SEQ ID NO: 37 - protein, 135 aa, Artificial sequence
  PD1 -10-5 (with CYS93>Ser substitution) 135 aa
PWNPPTFSPALLVVTEGDNATFTCSFSNTSESFVLNWYRMSPSNQTDKLAAFPEDRSQPGQDSRFRVTQLPNGRDFHMSVVRARRNDSGTYLCGAISLAPKAQIKESLRAELRVTERRAEVPTAHPSPSPRPAGQ

SEQ ID NO: 38 - protein, 128 aa, Artificial sequence
  PD1 -10-12 (with CYS93>Ser substitution) 128 aa
PWNPPTFSPALLVVTEGDNATFTCSFSNTSESFVLNWYRMSPSNQTDKLAAFPEDRSQPGQDSRFRVTQLPNGRDFHMSVVRARRNDSGTYLCGAISLAPKAQIKESLRAELRVTERRAEVPTAHPSP

SEQ ID NO: 39 - protein, 133 aa, Artificial sequence
  PD1 -5-12 (New, from V20) (with CYS93>Ser substitution) 133 aa
DSPDRPWNPPTFSPALLVVTEGDNATFTCSFSNTSESFVLNWYRMSPSNQTDKLAAFPEDRSQPGQDSRFRVTQLPNGRDFHMSVVRARRNDSGTYLCGAISLAPKAQIKESLRAELRVTERRAEVPTAHPSP

SEQ ID NO: 40 - protein, 127 aa, Artificial sequence
  PD1 -11-12 127 aa
WNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSP

SEQ ID NO: 41 - protein, 127 aa, Artificial sequence
  PD1 -11-12 (with CYS93>Ser substitution) 127 aa
WNPPTFSPALLVVTEGDNATFTCSFSNTSESFVLNWYRMSPSNQTDKLAAFPEDRSQPGQDSRFRVTQLPNGRDFHMSVVRARRNDSGTYLCGAISLAPKAQIKESLRAELRVTERRAEVPTAHPSP

SEQ ID NO: 42 - protein, 134 aa, Artificial sequence
  PD1 -11-5  134 aa
WNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQ

SEQ ID NO: 43 - protein, 134 aa, Artificial sequence
  PD1 -11-5  (with CYS93>Ser substitution) 134 aa
WNPPTFSPALLVVTEGDNATFTCSFSNTSESFVLNWYRMSPSNQTDKLAAFPEDRSQPGQDSRFRVTQLPNGRDFHMSVVRARRNDSGTYLCGAISLAPKAQIKESLRAELRVTERRAEVPTAHPSPSPRPAGQ

SEQ ID NO: 44 - protein, 138 aa, Artificial sequence
  PD1 -5-7 138 aa
DSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPA

SEQ ID NO: 45 - protein, 138 aa, Artificial sequence
  PD1 -5-7 (with CYS93>Ser substitution) 138 aa
DSPDRPWNPPTFSPALLVVTEGDNATFTCSFSNTSESFVLNWYRMSPSNQTDKLAAFPEDRSQPGQDSRFRVTQLPNGRDFHMSVVRARRNDSGTYLCGAISLAPKAQIKESLRAELRVTERRAEVPTAHPSPSPRPA

SEQ ID NO: 46 - protein, 136 aa, Artificial sequence
  PD1 -5-9 (with out CYS93>Ser substitution)136 aa
DSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPR

SEQ ID NO: 47 - protein, 145 aa, Artificial sequence
  PD1 -0-5 from (with out CYS93>Ser substitution)145 aa
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQ

SEQ ID NO: 48 - protein, 143 aa, Artificial sequence
  PD1 -0-7 from (with out CYS93>Ser substitution) 143 aa
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPA

SEQ ID NO: 49 - protein, 356 aa, Artificial sequence
  PD1-4-1BBL FUSION 356 aa
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVGACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 50 - protein, 332 aa, Artificial sequence
  PD1-4-1BBL FUSION (variant1) 332 aa
DSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 51 - protein, 338 aa, Artificial sequence
  PD1-4-1BBL FUSION (variant2) 338 aa
DSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 52 - protein, 326 aa, Artificial sequence
  PD1-4-1BBL FUSION (variant3) 326 aa
DSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQGLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 53 - protein, 340 aa, Artificial sequence
  PD1-4-1BBL FUSION (variant4) 340 aa
DSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQGSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 54 - protein, 333 aa, Artificial sequence
  PD1-4-1BBL FUSION (variant5) 333 aa
DSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPGSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 55 - protein, 325 aa, Artificial sequence
  PD1-4-1BBL FUSION (variant6) 325 aa
DSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 56 - protein, 331 aa, Artificial sequence
  PD1-4-1BBL FUSION (variant7) 331 aa
DSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 57 - protein, 319 aa, Artificial sequence
  PD1-4-1BBL FUSION (variant8) 319 aa
DSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPGLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 58 - protein, 328 aa, Artificial sequence
  PD1-4-1BBL FUSION (variant9) 328 aa
PWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPGSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 59 - protein, 320 aa, Artificial sequence
  PD1-4-1BBL FUSION (variant10) 320 aa
PWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 60 - protein, 326 aa, Artificial sequence
  PD1-4-1BBL FUSION (variant11) 326 aa
PWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 61 - protein, 314 aa, Artificial sequence
  PD1-4-1BBL FUSION (variant12) 314 aa
PWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPGLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 62 - protein, 335 aa, Artificial sequence
  PD1-4-1BBL FUSION (variant13) 335 aa
PWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQGSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 63 - protein, 327 aa, Artificial sequence
  PD1-4-1BBL FUSION (variant14) 327 aa
PWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 64 - protein, 333 aa, Artificial sequence
  PD1-4-1BBL FUSION (variant15) 333 aa
PWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 65 - protein, 321 aa, Artificial sequence
  PD1-4-1BBL FUSION (variant16) 321 aa
PWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQGLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 66 - protein, 332 aa, Artificial sequence
  PD1-4-1BBL FUSION (variant17) 332 aa
DSPDRPWNPPTFSPALLVVTEGDNATFTCSFSNTSESFVLNWYRMSPSNQTDKLAAFPEDRSQPGQDSRFRVTQLPNGRDFHMSVVRARRNDSGTYLCGAISLAPKAQIKESLRAELRVTERRAEVPTAHPSPSPRPAGQGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 67 - protein, 319 aa, Artificial sequence
  PD1-4-1BBL FUSION (variant18) 319 aa
WNPPTFSPALLVVTEGDNATFTCSFSNTSESFVLNWYRMSPSNQTDKLAAFPEDRSQPGQDSRFRVTQLPNGRDFHMSVVRARRNDSGTYLCGAISLAPKAQIKESLRAELRVTERRAEVPTAHPSPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 68 - protein, 326 aa, Artificial sequence
  PD1-4-1BBL FUSION (variant19)
WNPPTFSPALLVVTEGDNATFTCSFSNTSESFVLNWYRMSPSNQTDKLAAFPEDRSQPGQDSRFRVTQLPNGRDFHMSVVRARRNDSGTYLCGAISLAPKAQIKESLRAELRVTERRAEVPTAHPSPSPRPAGQGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 69 - protein, 325 aa, Artificial sequence
  PD1-4-1BBL FUSION (variant20) 325 aa
DSPDRPWNPPTFSPALLVVTEGDNATFTCSFSNTSESFVLNWYRMSPSNQTDKLAAFPEDRSQPGQDSRFRVTQLPNGRDFHMSVVRARRNDSGTYLCGAISLAPKAQIKESLRAELRVTERRAEVPTAHPSPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 70 - protein, 330 aa, Artificial sequence
  PD1-4-1BBL FUSION (variant21) 320 aa
DSPDRPWNPPTFSPALLVVTEGDNATFTCSFSNTSESFVLNWYRMSPSNQTDKLAAFPEDRSQPGQDSRFRVTQLPNGRDFHMSVVRARRNDSGTYLCGAISLAPKAQIKESLRAELRVTERRAEVPTAHPSPSPRPAGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 71 - protein, 356 aa, Artificial sequence
  PD1-4-1BBL FUSION (variant22) 362 aa
PGWFLDSPDRPWNPPTFSPALLVVTEGDNATFTCSFSNTSESFVLNWYRMSPSNQTDKLAAFPEDRSQPGQDSRFRVTQLPNGRDFHMSVVRARRNDSGTYLCGAISLAPKAQIKESLRAELRVTERRAEVPTAHPSPSPRPAGQFQTLVGACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 72 - protein, 330 aa, Artificial sequence
  PD1-4-1BBL FUSION (variant23) 330 aa
DSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 73 - protein, 321 aa, Artificial sequence
  PD1-4-1BBL FUSION (variant24) 321 aa
DSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRGREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 74 - protein, 319 aa, Artificial sequence
  PD1-4-1BBL FUSION (variant25) 319 aa
DSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRGGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 75 - protein, 329 aa, Artificial sequence
  PD1-4-1BBL FUSION (variant26) 329 aa
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPA

SEQ ID NO: 76 - protein, 320 aa, Artificial sequence
  PD1-4-1BBL FUSION (variant27) 320 aa
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPA

SEQ ID NO: 77 - protein, 346 aa, Artificial sequence
  PD1-4-1BBL FUSION (variant28) 346 aa
DSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQGACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 78 - protein, 350 aa, Artificial sequence
  PD1-4-1BBL FUSION (variant29) 350 aa
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVGSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 79 - protein, 365 aa, Artificial sequence
  PD1-4-1BBL FUSION (variant30) 365 aa
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVGGGGSGGGGSACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 80 - protein, 750 aa, Artificial sequence
  PD1-4-1BBL FUSION (variant31) 750 aa
DSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQGGGGSGGGGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 81 - protein, 331 aa, Artificial sequence
  PD1-4-1BBL FUSION (variant32) 331 aa
AASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQ

SEQ ID NO: 82 - protein, 504 aa, Artificial sequence
  aa sequence of full length SIRP 504 aa
MEPAGPAPGRLGPLLCLLLAASCAWSGVAGEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYIVVGVVCTLLVALLMAALYLVRIRQKKAQGSTSSTRLHEPEKNAREITQDTNDITYADLNLPKGKKPAPQAAEPNNHTEYASIQTSPQPASEDTLTYADLDMVHLNRTPKQPAPKPEPSFSEYASVQVPRK

SEQ ID NO: 83 - protein, 343 aa, Artificial sequence
  ORIGINAL SIRPa DOMAIN (343AA)
EEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIY

SEQ ID NO: 84 - protein, 343 aa, Artificial sequence
  343 amino acids sequence of SIRPa with 4 point mutations
EEEIQVIQPDKSVLVAAGETATLRCTITSLIPVGPIQWFRGAGPGRVLIYNQKEGHFPRVTTVSDLTKRNNMDFSIRIGNITPADAGTYYCIKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIY

SEQ ID NO: 85 - protein, 116 aa, Artificial sequence
  116 aa SIRPa segment
EEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRA

SEQ ID NO: 86 - protein, 116 aa, Artificial sequence
  116 amino acids sequence of SIRPa with 4 point mutations
EEEIQVIQPDKSVLVAAGETATLRCTITSLIPVGPIQWFRGAGPGRVLIYNQKEGHFPRVTTVSDLTKRNNMDFSIRIGNITPADAGTYYCIKFRKGSPDDVEFKSGAGTELSVRA

SEQ ID NO: 87 - protein, 549 aa, Artificial sequence
  SIRPa-4-1BBL FUSION 549 aa
EEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYGACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 88 - protein, 543 aa, Artificial sequence
  SIRPa-4-1BBL FUSION (variant1) 543 aa
EEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYGSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 89 - protein, 535 aa, Artificial sequence
  SIRPa-4-1BBL FUSION (variant2) 535 aa
EEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 90 - protein, 322 aa, Artificial sequence
  SIRPa-4-1BBL FUSION (variant3) 322 aa
EEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAGACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 91 - protein, 316 aa, Artificial sequence
  SIRPa-4-1BBL FUSION (variant3.1) 316 aa
EEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAGSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 92 - protein, 543 aa, Artificial sequence
  SIRPa-4-1BBL FUSION (variant1Mut) 543 aa
EEEIQVIQPDKSVLVAAGETATLRCTITSLIPVGPIQWFRGAGPGRVLIYNQKEGHFPRVTTVSDLTKRNNMDFSIRIGNITPADAGTYYCIKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYGSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 93 - protein, 535 aa, Artificial sequence
  SIRPa-4-1BBL FUSION (variant2Mut) 535 aa
EEEIQVIQPDKSVLVAAGETATLRCTITSLIPVGPIQWFRGAGPGRVLIYNQKEGHFPRVTTVSDLTKRNNMDFSIRIGNITPADAGTYYCIKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 94 - protein, 322 aa, Artificial sequence
  SIRPa-4-1BBL FUSION (variant3Mut) 322 aa
EEEIQVIQPDKSVLVAAGETATLRCTITSLIPVGPIQWFRGAGPGRVLIYNQKEGHFPRVTTVSDLTKRNNMDFSIRIGNITPADAGTYYCIKFRKGSPDDVEFKSGAGTELSVRAGACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 95 - protein, 316 aa, Artificial sequence
  SIRPa-4-1BBL FUSION (variant3.1Mut) 316 aa
EEEIQVIQPDKSVLVAAGETATLRCTITSLIPVGPIQWFRGAGPGRVLIYNQKEGHFPRVTTVSDLTKRNNMDFSIRIGNITPADAGTYYCIKFRKGSPDDVEFKSGAGTELSVRAGSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 96 - protein, 541 aa, Artificial sequence
  SIRPa-4-1BBL FUSION (variant4) 541 aa
EEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 97 - protein, 529 aa, Artificial sequence
  SIRPa-4-1BBL FUSION (variant5) 529 aa
EEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYGLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 98 - protein, 308 aa, Artificial sequence
  SIRPa-4-1BBL FUSION (variant6) 308 aa
EEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 99 - protein, 314 aa, Artificial sequence
  SIRPa-4-1BBL FUSION (variant7) 314 aa
EEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 100 - protein, 302 aa, Artificial sequence
  SIRPa-4-1BBL FUSION (variant8) 302 aa
EEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAGLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 101 - protein, 535 aa, Artificial sequence
  SIRPa-4-1BBL FUSION (variant2.1) 535 aa
SAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIY

SEQ ID NO: 102 - protein, 15 aa, Artificial sequence
  TCR sequence
CSARDPPSGRNGYTF

SEQ ID NO: 103 - protein, 13 aa, Artificial sequence
  TCR sequence
CASSPTIEETQYF

SEQ ID NO: 104 - protein, 12 aa, Artificial sequence
  TCR sequence
CASRSSNQPQHF

SEQ ID NO: 105 - protein, 15 aa, Artificial sequence
  TCR sequence
CASSPRDSWTGELFF

SEQ ID NO: 106 - protein, 15 aa, Artificial sequence
  TCR sequence
CASSANRAPNEKLFF

SEQ ID NO: 107 - protein, 15 aa, Artificial sequence
  TCR sequence
CASRTTSGEQETQYF

SEQ ID NO: 108 - protein, 15 aa, Artificial sequence
  TCR sequence
CASSYQVGDLGELFF

SEQ ID NO: 109 - protein, 17 aa, Artificial sequence
  TCR sequence
CASSPPSGAVRTDTQYF

SEQ ID NO: 110 - protein, 15 aa, Artificial sequence
  TCR sequence
CASSLATSSYNEQFF

SEQ ID NO: 111 - protein, 13 aa, Artificial sequence
  TCR sequence
CASSRLGGLTQYF

SEQ ID NO: 112 - protein, 15 aa, Artificial sequence
  TCR sequence
CASSQSATGNYEQYF

SEQ ID NO: 113 - protein, 20 aa, Artificial sequence
  TCR sequence
CASSYSIQGGMLSLGEKLFF

SEQ ID NO: 114 - protein, 12 aa, Artificial sequence
  TCR sequence
CATGLSVDTQYF

SEQ ID NO: 115 - protein, 14 aa, Artificial sequence
  TCR sequence
CASSTAGAGYEQYF

SEQ ID NO: 116 - protein, 15 aa, Artificial sequence
  TCR sequence
CASSITGGSGNEQFF

SEQ ID NO: 117 - protein, 14 aa, Artificial sequence
  TCR sequence
CASSDRGMNTEAFF

SEQ ID NO: 118 - protein, 14 aa, Artificial sequence
  TCR sequence
CASSFSAAQETQYF

SEQ ID NO: 119 - protein, 12 aa, Artificial sequence
  TCR sequence
CASSRDREKLFF

SEQ ID NO: 120 - protein, 11 aa, Artificial sequence
  TCR sequence
CASSKNNEQFF

SEQ ID NO: 121 - protein, 15 aa, Artificial sequence
  TCR sequence
CASSEGAGAKQPQHF

SEQ ID NO: 122 - protein, 16 aa, Artificial sequence
  TCR sequence
CASSLGGLAITDTQYF

SEQ ID NO: 123 - protein, 15 aa, Artificial sequence
  TCR sequence
CASSQMQGLVGEQYF

SEQ ID NO: 124 - protein, 14 aa, Artificial sequence
  TCR sequence
CASGDRGPRYEQYF

SEQ ID NO: 125 - protein, 16 aa, Artificial sequence
  TCR sequence
CSAGTGTSGGRETQYF

SEQ ID NO: 126 - protein, 17 aa, Artificial sequence
  TCR sequence
CASSSYTAKGRYNEQFF

SEQ ID NO: 127 - protein, 13 aa, Artificial sequence
  TCR sequence
CSASPDSNQPQHF

SEQ ID NO: 128 - protein, 14 aa, Artificial sequence
  TCR sequence
CASKVFSYTGELFF

SEQ ID NO: 129 - protein, 13 aa, Artificial sequence
  TCR sequence
CASSFEGYNEQFF

SEQ ID NO: 130 - protein, 14 aa, Artificial sequence
  TCR sequence
CASSLGDANNEQFF

SEQ ID NO: 131 - protein, 15 aa, Artificial sequence
  TCR sequence
CASSSTIGGVNEQFF

SEQ ID NO: 132 - protein, 17 aa, Artificial sequence
  TCR sequence
CASSLTGRAYMDYGYTF

SEQ ID NO: 133 - protein, 15 aa, Artificial sequence
  TCR sequence
CASSRALRGHNGYTF

SEQ ID NO: 134 - protein, 14 aa, Artificial sequence
  TCR sequence
CASSQVASTDTQYF

SEQ ID NO: 135 - protein, 15 aa, Artificial sequence
  TCR sequence
CASSSRDRMNTEAFF

SEQ ID NO: 136 - protein, 16 aa, Artificial sequence
  TCR sequence
CASSCMGQGEGQPQHF

SEQ ID NO: 137 - protein, 13 aa, Artificial sequence
  TCR sequence
CASSQQENQPQHF

SEQ ID NO: 138 - protein, 15 aa, Artificial sequence
  TCR sequence
CASSANSGNQETQYF

SEQ ID NO: 139 - protein, 14 aa, Artificial sequence
  TCR sequence
CASSLQSRSGEQYF

SEQ ID NO: 140 - protein, 14 aa, Artificial sequence
  TCR sequence
CASSLTSATGELFF

SEQ ID NO: 141 - protein, 16 aa, Artificial sequence
  TCR sequence
CASSLGGLAANYEQYF

SEQ ID NO: 142 - protein, 14 aa, Artificial sequence
  TCR sequence
CASSLAVQGGGYTF

SEQ ID NO: 143 - protein, 16 aa, Artificial sequence
  TCR sequence
CASSGGGLAGNSEQYF

SEQ ID NO: 144 - protein, 14 aa, Artificial sequence
  TCR sequence
CASTLGQGAWPLHF

SEQ ID NO: 145 - protein, 15 aa, Artificial sequence
  TCR sequence
CASSVRPTDTDTQYF

SEQ ID NO: 146 - protein, 13 aa, Artificial sequence
  TCR sequence
CASSVGGNTEAFF

SEQ ID NO: 147 - protein, 12 aa, Artificial sequence
  TCR sequence
CASSGDYDEQFF

SEQ ID NO: 148 - protein, 15 aa, Artificial sequence
  TCR sequence
CASSIRSGRVDEQFF

SEQ ID NO: 149 - protein, 14 aa, Artificial sequence
  TCR sequence
CASSKTDSNQPQHF